(19) United States
(12) Patent Application Publication
(10) Pub. No.: US 2009/0054403 A1
(43) Pub. Date: Feb. 26, 2009

(54) Treatment of Post-Traumatic Stress Disorder

(75) Inventors: Tom Woiwode, South San Francisco, CA (US); Mark Moran, South San Francisco, CA (US)

Correspondence Address: FENWICK & WEST LLP, SILICON VALLEY CENTER, 801 CALIFORNIA STREET, MOUNTAIN VIEW, CA, 94041, US

(73) Assignee: SYNOSIA THERAPEUTICS, South San Francisco, CA (US)

(21) Appl. No.: 12/178,513

(22) Filed: Jul. 23, 2008

Related U.S. Application Data
(60) Provisional application No. 60/935,036, filed on Jul. 23, 2007.

(51) Int. Cl. A61K 31/55; A61K 31/4164; A61K 31/4525

(52) U.S. Cl. 514/214.02; 514/392; 514/321

(57) ABSTRACT

Provided are methods of treating a patient diagnosed with post-traumatic stress disorder, by administering to the patient a therapeutically effective amount of Compound A. Also provided are methods of improving resilience in a patient by administering a therapeutically effective amount of Compound A. Also provided are methods of diagnosing post-traumatic stress disorder in a patient by administering to the patient a therapeutically effective amount of Compound A and assessing at least one of sign, symptom, or symptom cluster of post-traumatic stress disorder; and diagnosing post-traumatic stress disorder in the patient if the Compound A reduces at least one of sign, symptom, and symptom cluster of post-traumatic stress disorder.

### CROSS-REFERENCE TO RELATED APPLICATIONS

This application claims the benefit of and priority under 35 U.S.C. § 119(e) to U.S. Provisional Patent Application Ser. No. 60/935,036, “TREATMENT OF POST-TRAUMATIC STRESS DISORDER” filed Jul. 23, 2007, which is incorporated herein by reference in its entirety.

### FIELD OF INVENTION

This relates generally to methods for treating post-traumatic stress disorder and more particularly methods of treating post-traumatic stress disorder using compound A, an inhibiting dopamine β-hydroxylase. Also provided are methods of improving resilience in a patient by administering a therapeutically effective amount of Compound A. Also provided are methods of diagnosing post-traumatic stress disorder in a patient by administering to the patient a therapeutically effective amount of Compound A and assessing at least one of sign, symptom, or symptom cluster of post-traumatic stress disorder; and diagnosing post-traumatic stress disorder in the patient if the Compound A reduces at least one of sign, symptom, and symptom cluster of post-traumatic stress disorder.

### BACKGROUND OF THE INVENTION

Anxiety disorders are the most commonly occurring disorders of the psychiatric illnesses with an immense economic burden. In addition to generalized anxiety disorder, they encompass post-traumatic stress disorder, panic disorder, obsessive compulsive disorder and social as well as other phobias.

Post-traumatic stress disorder can be severe and chronic, with some studies suggesting a lifetime prevalence of 1.3% to 7.8% in the general population. Post-traumatic stress disorder typically follows a psychologically distressing traumatic event. These events may include military combat, terrorist incidents, physical assault, sexual assault, motor vehicle accidents, and natural disasters, for example. The response to the event can involve intense fear, helplessness, or horror. Most people recover from the traumatic event with time and return to normal life. In contrast, in post-traumatic stress disorder victims, symptoms persist and may worsen with time, preventing a return to normal life.

Psychotherapy is currently the backbone of post-traumatic disorder treatment. Methods include cognitive-behavioral therapy, exposure therapy, and eye movement desensitization and reprocessing. Medication can enhance the effectiveness of psychotherapy. Selective serotonin reuptake inhibitors (SSRIs), such as sertraline (Zoloft™) and paroxetine (Paxil®), are the only medications approved for treating PTSD by the Food and Drug Administration. Many unwanted side effects and characteristics are associated with SSRI usage. These include concerns about drug interactions, gastrointestinal side effects, sexual side effects, suicidal ideation, acute anxiogenic effects, and slow onset of action. Some tricyclic antidepressants (TCAs) and monamine oxidase inhibitors (MAOIs) appear to have some efficacy but patient tolerance is low due to the high incidence of side effects. MAOIs have dietary restriction requirements and are linked to hypertensive events. TCAs have anticholinergic and cardiovascular side effects. Lamotrigine, a sodium channel blocker, has had some efficacy in treating post-traumatic stress disorder in a small scale placebo controlled study. Difficulty in the use of lamotrigine due the to necessity for titration and the risk of developing Steven Johnson Syndrome, a life threatening rash, render it a poor candidate for therapeutic use.

There is a need for the development of treatments for post-traumatic stress disorder that are safe and effective.

Dopamine is a catecholamine neurotransmitter found predominately, along with specific dopaminergic receptors, in the central nervous system. Norepinephrine is a circulating catecholamine, which acts at adrenergic receptors in central and peripheral systems. Dopamine β-hydroxylase (DBH) catalyzes the conversion of dopamine to norepinephrine and is found in both central and peripheral sympathetic neurons. Inhibition of DBH concurrently elevates dopamine levels by blocking its metabolism and reduces norepinephrine levels by blocking its synthesis. Nepicastat ((S)-5-Aminomethyl-1-(5,7-difluoro-1,2,3,4-tetrahydronaphth-2-yl)-2,3-dihydro-2-thioxo-1H-imidazole hydrochloride), is a DBH inhibitor.

### SUMMARY OF THE INVENTION

Provided herein are methods of treating a patient diagnosed with post-traumatic stress disorder, by administering to the patient a therapeutically effective amount of Compound A.

Also provided are methods of improving resilience in a patient by administering a therapeutically effective amount of Compound A.

Also provided are methods of diagnosing post-traumatic stress disorder in a patient by administering to the patient a therapeutically effective amount of Compound A and assessing at least one of sign, symptom, or symptom cluster of post-traumatic stress disorder; and diagnosing post-traumatic stress disorder in the patient if the Compound A reduces at least one of sign, symptom, and symptom cluster of post-traumatic stress disorder.

### DESCRIPTION OF DRAWINGS

FIG. 1 Shows Details of the individual enzymatic assays.

FIG. 2 Shows the Effects of Nepicastat on Tissue Noradrenaline and Dopamine Content in the mesentec artery (a), left ventricle (b) and cerebral cortex (c) of SHRs.

FIG. 3 Shows the Effects of Nepicastat Tissue Dopamine/Noradreline ration in the mesenteric artery (a), left ventricle (b), and cerebral cortex (c) of SHRs.

FIG. 4 Shows the Effects Nepicastat on Tissue Noradrenaline and Dopamine Content in renal artery, left ventricle, and cerebral cortex of beagle dogs.

FIG. 5 Shows the Effects of Nepicastat on Tissue Dopamine/Noradrenaline ratio in the renal artery, left ventricle, and cerebral cortex of beagle dogs.

FIG. 6 Shows the Effects of Nepicastat on Plasma Concentrations of Noradrenaline (a), Dopamine (b), and Dopamine/Noradrenaline ratio (c) in beagle dogs.

FIG. 7 Shows the Effect of Nepicastat and (R)-5-Aminomethyl-1-(5,7-difluoro-1,2,3,4-tetrahydronaphth-2-yl)-2,3-dihydro-2-thioxo-1H-imidazole hydrochloride, at 30 mg.kg⁻¹; po, on noradrenaline content, dopamine content and dopamine/noradrenaline ratio in mesenteric artery, left ventricle and cerebral cortex of SHRs.

FIG. 8 Shows Structures of 1, 2a (nepicastat), and 2b ((R)-5-Aminomethyl-1-(5,7-difluoro-1,2,3,4-tetrahydronaphth-2-yl)-2,3-dihydro-2-thioxo-1H-imidazole hydrochloride).

FIG. 9 Shows Chemical Scheme.

FIG. 10 Shows a Table Describing the Nepicastat Interaction of Nepicastat at DBH and a range of selected enzymes and receptors.

FIG. 11 Shows Effects of Nepicastat on tissue DA/NE Ratio in SHRs (A) and normal beagle dogs (B).

FIG. 12 Shows Effects of Chronic Administration of Nepicastat on plasma DA/NE ration in normal beagle dogs.

FIG. 13 Shows Effects of Orally administered Nepicastat on mean arterial pressure in SHR.

FIG. 14 Shows the Concentrations (pg/ml) of the Free Base of Norepinephrine and Dopamine in Samples of Plasma Collected from a Peripheral Vein of Supine CHF Patients During Daily Oral Administration of Placebo for 3 Months.

FIG. 15 Shows the Concentrations (pg/ml) of the Free Base of Norepinephrine and Dopamine in Samples of Plasma Collected from a Peripheral Vein of Supine CHF Patients During Daily Oral Administration of 20 mg of Nepicastat Free Base for 3 Months.

FIG. 16 Shows the Concentrations (pg/ml) of the Free Base of Norepinephrine and Dopamine in Samples of Plasma Collected from a Peripheral Vein of Supine CHF Patients During Daily Oral Administration of 40 mg of Nepicastat Free Base for 3 Months.

FIG. 17 Shows the Concentrations (pg/ml) of the Free Base of Norepinephrine and Dopamine in Samples of Plasma Collected from a Peripheral Vein of Supine CHF Patients During Daily Oral Administration of 60 mg of Nepicastat Free Base for 3 Months.

FIG. 18 Shows the Concentrations (pg/ml) of the Free Base of Norepinephrine and Dopamine in Samples of Plasma Collected from a Peripheral Vein of Supine CHF Patients During Daily Oral Administration of 80 mg of Nepicastat Free Base for 3 Months.

FIG. 19 Shows the Concentrations (pg/ml) of the Free Base of Norepinephrine and Dopamine in Samples of Plasma Collected from a Peripheral Vein of Supine CHF Patients During Daily Oral Administration of 120 mg of Nepicastat Free Base for 3 Months.

FIG. 20 Shows the Concentrations (pg/ml) of the Free Base of Norepinephrine in Samples of Plasma Collected from the Arterial Vein and Coronary Sinus of Catheterized CHF Patients During Daily Oral Administration of Placebo for 3 Months.

FIG. 21 Shows the Concentrations (pg/ml) of the Free Base of Dopamine in Samples of Plasma Collected from the Arterial Vein and Coronary Sinus of Catheterized CHF Patients During Daily Oral Administration of Placebo for 3 Months.

FIG. 22 Shows the Concentrations (pg/ml) of the Free Base of Norepinephrine in Samples of Plasma Collected from the Arterial Vein and Coronary Sinus of Catheterized CHF Patients During Daily Oral Administration of 20 mg of Nepicastat Free Base for 3 Months

FIG. 23 Shows the Concentrations (pg/ml) of the Free Base of Dopamine in Samples of Plasma Collected from the Arterial Vein and Coronary Sinus of Catheterized CHF Patients During Daily Oral Administration of 20 mg of Nepicastat Free Base for 3 Months.

FIG. 24 Shows the Concentrations (pg/ml) of the Free Base of Norepinephrine in Samples of Plasma Collected from the Arterial Vein and Coronary Sinus of Catheterized CHF Patients During Daily Oral Administration of 40 mg of Nepicastat Free Base for 3 Months.

FIG. 25 Shows the Concentrations (pg/ml) of the Free Base of Dopamine in Samples of Plasma Collected from the Arterial Vein and Coronary Sinus of Catheterized CHF Patients During Daily Oral Administration of 40 mg of Nepicastat Free Base for 3 Months

FIG. 26 Shows the Concentrations (pg/ml) of the Free Base of Norepinephrine in Samples of Plasma Collected from the Arterial Vein and Coronary Sinus of Catheterized CHF Patients During Daily Oral Administration of 60 mg of Nepicastat Free Base for 3 Months.

FIG. 27 Shows the Concentrations (pg/ml) of the Free Base of Dopamine in Samples of Plasma Collected from the Arterial Vein and Coronary Sinus of Catheterized CHF Patients During Daily Oral Administration of 60 mg of Nepicastat Free Base for 3 Months.

FIG. 28 denotes data that should be discounted from further statistical analysis together with the reason for such an action.

FIG. 29 Shows Pharmacokinetics Parameters of Nepicastat in Rats.

FIG. 30 Shows the Concentration of Nepicastat in Plasma of Male Rats Following a Single 10 mg/kg Oral Dose of Nepicastat.

FIG. 31 Shows the Concentration of Nepicastat in Plasma of Male Rats Following a Single 30 mg/kg Oral Dose of Nepicastat.

FIG. 32 Shows the Concentration of Nepicastat in Plasma of Male Rats Following a Single 100 mg/kg Oral Dose of Nepicastat.

FIG. 33 Shows the Concentration of Mean Concentration of Nepicastat in Plasma of Rats Following a Single 10, 30, or 100 mg/kg oral dose of Nepicastat. Values are the means of three rats per time point.

FIG. 34 Shows the Linear Relationship Between Dose and Values of AUC for Nepicastat in Plasma.

FIG. 35 Shows the Concentration of Nepicastat in Plasma of Female Rats Following a Single 30 mg/kg Oral Dose of Nepicastat.

FIG. 36 Shows the Mean Concentrations of Nepicastat in Plasma and Brain of Male Rats Following a Single 10 mg/kg oral dose of Nepicastat.

FIG. 37 Shows the Concentration of Nepicastat in Brain of Male Rats Following a Single 10 mg/kg Oral Dose of Nepicastat.

FIG. 38 Shows the Norepinephrine Concentration in the Mesenteric Artery.

FIG. 39 Shows the Dopamine Concentration in the Mesenteric Artery.

FIG. 40 Shows the Dopamine/Norepinephrine Concentration in the Mesenteric Artery.

FIG. 41 Shows the Norepinephrine Levels in the Rat Left Ventricle.

FIG. 42 Shows the Dopamine Levels in the Rat Left Ventricle.

FIG. 43 Shows the Dopamine/Norepinephrine Levels in the Rat Left Ventricle.

FIG. 44 Shows the Dopamine Concentration (μg/g wet weight) in the cerebral cortex of SHR plotted as a function of dose. Tissue was harvested six hours after the third of three oral doses administered 12 hours part. (n=9)

FIG. 45 Shows the Norepinephrine Concentration (μg/g wet weight) in the cerebral cortex of SHR plotted as a function of dose. Tissue was harvested six hours after the third of three oral doses administered 12 hours part. (n=9)

FIG. 46 Shows the Dopamine/Norepinephrine Concentration (μg/μg wet weight) in the cerebral cortex of SHR plotted as a function of dose. Tissue was harvested six hours after the third of three oral doses administered 12 hours part. (n=9)

FIG. 47 Shows the Dopamine Concentration (μg/g wet weight) in the left ventricle of SHR plotted as a function of dose. Tissue was harvested six hours after the third of three oral doses administered 12 hours part. (n=9)

FIG. 48 Shows the Norepinephrine Concentration (μg/g wet weight) in the left ventricle of SHR plotted as a function of dose. Tissue was harvested six hours after the third of three oral doses administered 12 hours part. (n=9)

FIG. 49 Shows the Dopamine/Norepinephrine Concentration (μg/μg wet weight) in the left ventricle of SHR plotted as a function of dose. Tissue was harvested six hours after the third of three oral doses administered 12 hours part. (n=9)

FIG. 50 Shows the Dopamine Concentration (μg/g wet weight) in the Mesenteric artery of SHR plotted as a function of dose. Tissue was harvested six hours after the third of three oral doses administered 12 hours part. (n=9)

FIG. 51 Shows the Norepinephrine Concentration (μg/g wet weight) in the Mesenteric artery of SHR plotted as a function of dose. Tissue was harvested six hours after the third of three oral doses administered 12 hours part. (n=9)

FIG. 52 Shows the Dopamine/Norepinephrine Concentration (μg/μg wet weight) in the Mesenteric artery of SHR plotted as a function of dose. Tissue was harvested six hours after the third of three oral doses administered 12 hours part. (n=9)

FIG. 53 Shows Dopamine concentration (μg/g wet weight) in the cerebral cortex of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9)

FIG. 54 Shows Norepinephrine (μg/g wet weight) in the cerebral cortex of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9)

FIG. 55 Shows the Dopamine/Norepinephrine Concentration (μg/μg wet weight) in the cerebral cortex of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9).

FIG. 56 Shows the Dopamine Concentration (μg/g wet weight) in the left ventricle of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9)

FIG. 57 Shows the Norepinephrine Concentration (μg/g wet weight) in the left ventricle of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9)

FIG. 58 Shows the Dopamine/Norepinephrine Concentration (μg/μg wet weight) in the left ventricle of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9).

FIG. 59 Shows Dopamine Concentration (μg/g wet weight) in the Mesenteric Artery of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9).

FIG. 60 Shows Norepinephrine Concentration (μg/g wet weight) in the Mesenteric Artery of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9).

FIG. 61 Shows Dopamine/Norepinephrine Concentration (μg/μg wet weight) in the Mesenteric Artery of SHR following administration of Nepicastat, (R)-5-Aminomethyl-1-(5,7-difluoro-1,2,3,4-tetrahydronaphth-2-yl)-2,3-dihydro-2-thioxo-1H-imidazole hydrochloride, or dH20 vehicle, or SKF102698 or PEG 400:dH20 (1:1) vehicle. Tissue was harvested six hours after the third of three oral doses administered 12 hours apart. (n=9).

FIG. 62 Shows the Catecholamine levels in the cortex, striatum, and mesenteric artery.

FIG. 63 Shows the Triiodothyronine levels in serum.

FIG. 64 Shows the Thyroxine levels in serum.

FIG. 65 Shows the Concentrations of Dopamine and Norepinephrine in Dog Kidney Medulla in Response to Nepicastat.

FIG. 66 Shows the Concentration of Dopamine and Norepinephrine in Dog Kidney Cortex in Response to Nepicastat.

FIG. 67 Shows the Effect of placebo or Nepicastat on plasma DA levels pg/ml) in normal beagle dogs.

FIG. 68 Shows the Effect of placebo or Nepicastat on plasma NE levels (pg/ml) in normal beagle dogs.

FIG. 69 Shows the Effect of placebo or Nepicastat on plasma DA/NE ratio in normal beagle dogs.

FIG. 70 Shows the Effect of placebo or Nepicastat on plasma EPI levels (pg/ml) in normal beagle dogs.

FIG. 71 Shows the Effects of chronic administration (14.5 days) of Nepicastat on plasma levels of NE, DA and EPI in normal beagle dogs. N=8 per group. *<0.05 vs. placebo

FIG. 72 Shows the Effect Concentrations (ng/ml) of the free base of Nepicastat and RS 47831 in samples of plasma collected following oral administration of RS 25560-197 (2 mg/kg; bid) to beagle dogs for 14.5 days.

FIG. 73 Shows the Dopamine Levels in the Renal Artery in Dogs.

FIG. 74 Shows the Norepinephrine Levels in the Renal Artery in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 75 Shows the Dopamine Levels in the Renal Artery in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 76 Shows the Dopamine Levels in the Cerebral Cortex in Dogs. Dogs were orally administered 0, 5, 15, or 30 mg/kg capsules b.i.d. for 4.5 days and tissue was harvested 6 hr after the final administration. N=8, *p<00.1; M 0.05<p<0.10 vs. placebo (mean±SD).

FIG. 77 Shows the Norepinephrine Levels in the Cerebral Cortex in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 78 Shows the Dopamine/Norepi ratio Levels in the Cerebral Cortex in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 79 Shows the Dopamine Levels in the Left Ventricle in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 80 Shows the Norepinephrine Levels in the Left Ventricle in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 81 Shows the Dopamine/Norepi Levels in the Left Ventricle in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 82 Shows the Dopamine Levels in the Renal Cortex in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 83 Shows the Norepinephrine Levels in the Renal Cortex in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 84 Shows the Dopamine/Norepi Levels in the Renal Cortex in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 85 Shows the Dopamine Levels in the Renal Medulla in Dogs. Dogs were orally administered 0, 5, 15, or 30 mg/kg capsules b.i.d. for 4.5 days and tissue was harvested 6 hr after the final administration. N=8, *p<0.01; M, 0.05<p<0.10 vs. placebo (mean±SD).

FIG. 86 Shows the Norepinephrine Levels in the Renal Medulla in Dogs. Dogs were orally administered 0, 5, 15, or 30 mg/kg capsules b.i.d. for 4.5 days and tissue was harvested 6 hr after the final administration. N=8, *p<0.01; M, 0.05<p<0.10 vs. placebo (mean±SD).

FIG. 87 Shows the Dopamine/Norepi Levels in the Renal Medulla in Dogs. Dogs were orally administered 0, 5, 15, or 30 mg/kg capsules b.i.d. for 4.5 days and tissue was harvested 6 hr after the final administration. N=8, *p<0.01 vs. placebo (mean±SD).

FIG. 88 Shows the Tissue Concentration of Nepicastat. Dogs were orally administered 0, 5, 15, or 30 mg/kg capsules b.i.d. for 4.5 days and tissue was harvested 6 hr after the final administration. N=8 (means only).

FIG. 89 Shows Day 5 plasma concentrations of Nepicastat. Dogs were orally administered 0, 5, 15, or 30 mg/kg capsules b.i.d. for 4.5 days and tissue was harvested 6 hr after the final administration. N=8 (means only).

FIG. 90 Shows Area Under the Curve (AUC) for 0-8 hr of Day 4 Plasma concentrations of Nepicastat. Dogs were orally administered 0, 5, 15, or 30 mg/kg capsules b.i.d. N=8 (means only).

FIG. 91 Shows the β-Adrenergic Receptor Binding Data.

FIG. 92 Shows the Effects of nepicastat on % inhibition of enzyme activity.

FIG. 93 Shows the activity of bovine DBH, expressed in the percent of inhibition, plotted as a function of the log of the inhibitor concentration.

FIG. 94 Shows the activity of human DBH, expressed in the percent of inhibition, plotted as a function of the log of the inhibitor concentration.

FIG. 95 Shows the IC50 of Three DBH Inhibitors on Bovine and Human DBH Activity (mean±SE).

FIG. 96 Shows the Lineweaver-Burk plot of the inhibition data against bovine DBH (A), and the plot of apparent KM versus inhibitor concentration (B).

FIG. 97 Shows the Outline of Studies for Determining Nepicastat Affinity in binding assays.

FIG. 98 Shows the Receptor Profile of Nepicastat.

FIG. 99 Shows the Summary of Rectal Temperature (Degrees Centigrade).

FIG. 100 Shows the Summary of Clinical Observations and Behavior Tests for Vehicle treated Animals.

FIG. 101 Shows the Summary of Clinical Observations and Behavior Tests for 30 mg/kg Nepicastat Treated Animals.

FIG. 102 Shows the Summary of Clinical Observations and Behavior Tests for 100 mg/kg Nepicastat Treated Animals.

FIG. 103 Shows the Summary of Clinical Observations and Behavior Tests for 300 mg/kg Nepicastat Treated Animals.

FIG. 104 Shows the Nepicastat Motor Activity Experiment: Horizontal Activity at 0.5 and 1 Hour.

FIG. 105 Shows the Nepicastat Motor Activity Experiment: Horizontal Activity at 1.5 and 2 Hours.

FIG. 106 Shows the Nepicastat Motor Activity Experiment: Horizontal Activity at 2.5 and 3 Hours.

FIG. 107 Shows the Nepicastat Motor Activity Experiment: Horizontal Activity at 3.5 and 4 Hours.

FIG. 108 Shows the Nepicastat Motor Activity Experiment: NO. of Movements at 0.5 and 1 Hour.

FIG. 109 Shows the Nepicastat Motor Activity Experiment: NO. of Movements at 1.5 and 2 Hours.

FIG. 110 Shows the Nepicastat Motor Activity Experiment: NO. of Movements at 2.5 and 3 Hours.

FIG. 111 Shows the Nepicastat Motor Activity Experiment: NO. of Movements at 3.5 and 4 Hours.

FIG. 112 Shows the Nepicastat Motor Activity Experiment: Rest Time (Seconds) 0.5 and 1 Hour.

FIG. 113 Shows the Nepicastat Motor Activity Experiment: Rest Time (Seconds) 1.5 and 2 Hours.

FIG. 114 Shows the Nepicastat Motor Activity Experiment: Rest Time (Seconds) 2.5 and 3 Hours.

FIG. 115 Shows the Nepicastat Motor Activity Experiment: Rest Time (Seconds) 3.5 and 4 Hours.

FIG. 116 Shows the DBHI Motor Activity Experiment: Horizontal Activity.

FIG. 117 Shows the DBHI Motor Activity Experiment: No. of Movements.

FIG. 118 Shows the DBHI Motor Activity Experiment: Rest Time (Seconds).

FIG. 119 Shows the Summary Statistics and Significance Assessments for Maximum Startle RESP.

FIG. 120 Shows the Summary Statistics and Significance Assessments for Maximum Startle RESP.

FIG. 121 Shows the Summary Statistics and Significance Assessments for Maximum Startle RESP.

FIG. 122 Shows the Summary Statistics and Significance Assessments for Maximum Startle RESP.

FIG. 123 Shows the Nepicastat and H20 Versus Time with Respect to St Max.

FIG. 124 Shows the Nepicastat and H20 Versus Time with Respect to St Avg.

FIG. 125 Shows the PEG and SKF Versus Time with Respect to St Max.

FIG. 126 Shows the PEG and SKF Versus Time with Respect to St Avg.

FIG. 127 Shows the Clonidine and H20 Versus Time with Respect to St Max.

FIG. 128 Shows the Clonidine and H20 Versus Time with Respect to St Avg.

FIG. 129 Shows the Pre-Treatment Acoustic Startle Reactivity and Starting Date for Each Rat.

FIG. 130 Shows the Pre-Treatment Acoustic Startle Reactivity and Starting Date for Each Rat.

FIG. 131 Shows the Lack of Effect of the DBHIs Nepicastat and SKF 102698 on Body Core Temperature.

FIG. 132 Shows the Mean Body Core Temperatures (° celcius) at Baseline and Day 1.

FIG. 133 Shows the Mean Body Core Temperatures (° celcius) at Day 5 and Day

FIG. 134 Shows the Effect of SKF 102698 Spontaneous Motor Activity.

FIG. 135 Shows Spontaneous Motor Activity at 0-15 and 15-30 minutes

FIG. 136 Shows Spontaneous Motor Activity at 30-45 and 45-60 minutes

FIG. 137 Shows the Lack of Effect of Nepicastat on Spontaneous Motor Activity.

FIG. 138 Shows the Lack of Effect of the DBHI Compounds SKF 102698 and Nepicastat on Pre-Pulse Inhibition.

FIG. 139 Shows the Summary Statistics and P-Values for Overall Pairwise Treatment Comparisons for Percent Prepulse Inhibition in Rats.

FIG. 140 Shows the Summary Statistics and P-Values for Pairwise Treatment Comparisons Within Time for Percent Prepulse Inhibition in Rats (for startles 1-15).

FIG. 141 Shows the Summary Statistics and P-Values for Pairwise Treatment Comparisons Within Time for Percent Prepulse Inhibition in Rats (for startles 31-45).

FIG. 142 Shows the Decrease in Acoustic Startle Reactivity Produced by the DBHI SKF-102698 but not by Nepicastat.

FIG. 143 Shows the Summary Statistics and P-Values for Overall Pairwise Treatment Comparisons for Acoustic Startle Reactivity in Rats.

FIG. 144 Shows the Summary Statistics and P-Values for Pairwise Treatment Comparisons Within Time for Acoustic Startle Reactivity in Rats (for startles 1-15).

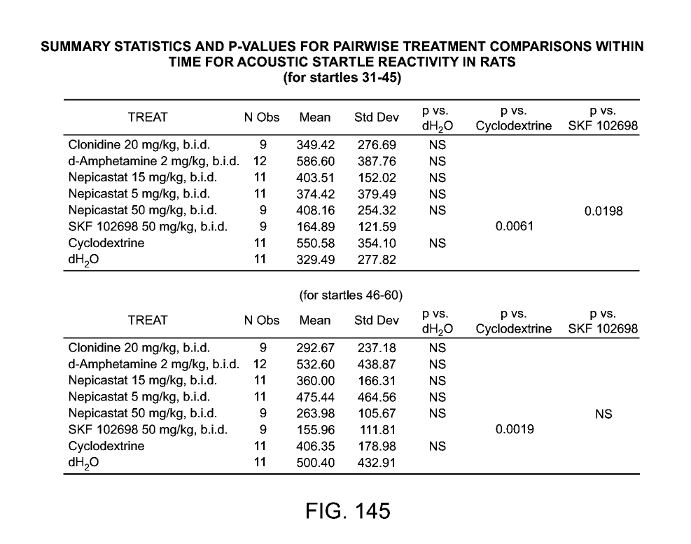

FIG. 145 Shows the Summary Statistics and P-Values for Pairwise Treatment Comparisons Within Time for Acoustic Startle Reactivity in Rats (for startles 31-45).

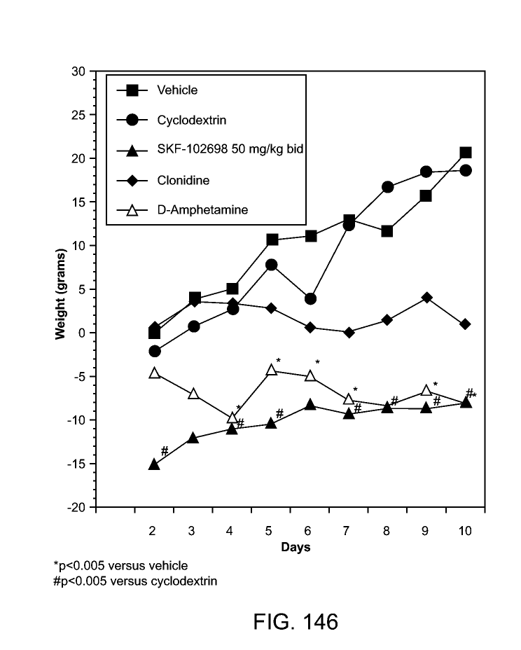

FIG. 146 Shows the Effect of SKF 102698 on Change of Body Weight.

FIG. 147 Shows the Lack of Effect of Nepicastat on Change of Body Weight.

FIG. 148 shows results of oral delivery in monkeys.

FIG. 149 shows results of oral delivery in monkeys.

FIG. 150 shows the clinical rating scale used in these studies.

FIG. 151 summarizes the lesioning schedules for animals in Groups A, B, C, and D.

FIG. 152 summarizes the lesioning schedules for animals in Groups A, B, C, and D.

FIG. 153 shows IRAM (A) and CRS (B) for placebo treatment

FIG. 154 shows IRAM (A) and CRS (B) for Group B.

FIG. 155 shows IRAM (A) and CRS (B) for Group C.

FIG. 156 shows IRAM (A) and CRS (B) for Group D.

FIG. 157 shows a comparison of placebo treatment to three concentrations of nepicastat.

FIG. 158 shows a comparison of placebo treatment to three concentrations of nepicastat.

FIG. 159 shows a comparison of post-MPTP-lesioned (pre-treatment) CRS to L-DOPA and placebo treatment for Group A.

FIG. 160 shows Friedman test and descriptive statistics for Group A.

FIG. 161 shows Dunnett's test post hoc analysis for Group A.

FIG. 162 shows a comparison of post-MPTP-lesioned (pre-treatment) CRS to L-DOPA and nepicastat treatment for Group B.

FIG. 163 shows Friedman test and descriptive statistics for Group B.

FIG. 164 shows Dunnett's test post hoc analysis for Group B.

FIG. 165 shows a comparison of post-MPTP-lesioned (pre-treatment) CRS to L-DOPA and nepicastat treatment for Group C.

FIG. 166 shows Friedman test and descriptive statistics for Group C.

FIG. 167 shows Dunnett's test post hoc analysis for Group C.

FIG. 168 shows a comparison of post-MPTP-lesioned (pre-treatment) CRS to L-DOPA and nepicastat treatment for Group D.

FIG. 169 shows Friedman test and descriptive statistics for Group D.

FIG. 170 shows Dunnett's test post hoc analysis for Group D.

FIG. 171 shows affinity counts measure for groups.

FIG. 172 descriptive statistics for treatment groups.

FIG. 173 shows the baseline heart rate and mean arterial pressure.

FIG. 174 shows the effect of nepicastat in heart rate in conscious SHR pretreated with SCH-23390 or vehicle.

FIG. 175 shows the effect of nepicastat on mean arterial pressure in SHR pretreated with SCH-23390 or vehicle.

FIG. 176 shows the mean blood pressures of the four groups of rats on the day prior to the start of the drug treatment.

FIG. 177 shows heart rates of the four groups of rats on the day prior to the start of the drug treatment.

FIG. 178 shows motor activities (in arbitrary units) of the four groups of rats on the day prior to the start of the drug treatment.

FIG. 179 shows mean blood pressures of the four groups of rats on day 1 of the drug treatments.

FIG. 180 shows mean blood pressures of the four groups of rats on day 2 of the drug treatments.

FIG. 181 shows mean blood pressures of the four groups of rats on day 3 of the drug treatments.

FIG. 182 shows mean blood pressures of the four groups of rats on day 7 of the drug treatments.

FIG. 183 shows heart rates of the four groups of rats on day 2 of the drug treatment.

FIG. 184 shows motor activities (in arbitrary units) of the four groups of rats on day 3 of the drug treatment.

FIG. 185 shows changes in body weights of the four groups of rats during the first 6 day treatment.

FIG. 186 shows the significance levels for each time point on mean blood pressure.

FIG. 187 shows the significance levels for each time point on mean blood pressure.

### DETAILED DESCRIPTION

As used herein, the following words and phrases are generally intended to have the meanings as set forth below, except to the extent that the context in which they are used indicates otherwise.

As used herein “Compound A” includes nepicastat (((S)-5-Aminomethyl-1-(5,7-difluoro-1,2,3,4-tetrahydronaphth-2-yl)-2,3-dihydro-2-thioxo-1H-imidazole hydrochloride)), ((R)-5-Aminomethyl-1-(5,7-difluoro-1,2,3,4-tetrahydronaphth-2-yl)-2,3-dihydro-2-thioxo-1H-imidazole hydrochloride), and mixtures thereof, as well as pharmaceutically acceptable salts thereof.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH2n-COOH where n is 0-4, and like salts. In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, the term “treating” refers to any manner in which at least one sign, symptom, or symptom cluster of a disease or disorder is beneficially altered so as to prevent or delay the onset, reduce the incidence or frequency, reduce the severity or intensity, retard the progression, prevent relapse, or ameliorate the symptoms or associated symptoms of the disease or disorder. For example, in post-traumatic stress disorder, treating the disorder can, in certain embodiments, cause a reduction in at least one of the frequency and intensity of at least one of a sign, symptom, and symptom cluster of post-traumatic stress disorder. As used herein the phrase “diagnosed with post-traumatic stress disorder (PTSD)” refers to having a sign, symptom, or symptom cluster indicative of post-traumatic stress disorder, a psychiatric disorder triggered by a traumatic event. Non-limiting examples of such traumatic events include military combat, terrorist incidents, physical assault, sexual assault, motor vehicle accidents, and natural disasters.

The Diagnostic and Statistical Manual of Mental Disorders-IV-Text revised (DSM-IV-TR), a handbook for mental health professionals that lists categories of mental disorders and the criteria, classifies post-traumatic stress disorder as an anxiety disorder. According to the DSM-IV-TR, a PTSD diagnosis can be made if:

1. the patient experienced, witnessed, or was confronted with an event or events that involved actual or threatened death or serious injury, or a threat to the physical integrity of self or others and the response involved intense fear, helplessness, or horror;

2. as a consequence of the traumatic event, the patient experiences at least 1 re-experiencing/intrusion symptom, 3 avoidance/numbing symptoms, and 2 hyperarousal symptoms, and the duration of the symptoms is for more than 1 month; and

3. the symptoms cause clinically significant distress or impairment in social, occupational, or other important areas of functioning.

In certain embodiments, if the patient's disorder fulfills DSM-IV-TR criteria, the patient is diagnosed with post-traumatic stress disorder. In certain embodiments, if the patient has at least one sign, symptom, or symptom cluster of post-traumatic stress disorder, the patient is diagnosed with post-traumatic stress disorder. In certain embodiments, a scale is used to measure a sign, symptom, or symptom cluster of post-traumatic stress disorder, and post-traumatic stress disorder is diagnosed on the basis of the measurement using that scale. In certain embodiments, a “score” on a scale is used to diagnose or assess a sign, symptom, or symptom cluster of post-traumatic stress disorder. In certain embodiments, a “score” can measure at least one of the frequency, intensity, or severity of a sign, symptom, or symptom cluster of post-traumatic stress disorder.

As used herein, the term “scale” refers to a method to measure at least one sign, symptom, or symptom cluster of post-traumatic stress disorder in a patient. In certain embodiments, a scale may be an interview or a questionnaire. Non-limiting examples of scales are Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

As used herein, the terms “sign” and “signs” refer to objective findings of a disorder. In certain embodiments, a sign can be a physiological manifestation or reaction of a disorder. In certain embodiments, a sign may refer to heart rate and rhythm, body temperature, pattern and rate of respiration, blood pressure. In certain embodiments, signs can be associated with symptoms. In certain embodiments, signs can be indicative of symptoms.

As used herein, the term “symptom” and “symptoms” refer to subjective indications that characterize a disorder. Symptoms of post-traumatic stress disorder may refer to, for example, but not limited to recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response. In certain embodiments, potentially threatening stimuli can cause hyperarousal or anxiety. In certain embodiments, the physiological reactivity manifests in at least one of abnormal respiration, abnormal cardiac rate of rhythm, abnormal blood pressure, abnormal function of a special sense, and abnormal function of sensory organ. In certain embodiments, restricted range of effect characterized by diminished or restricted range or intensity of feelings or display of feelings can occur and s sense of a foreshortened future can manifest in thinking that one will not have a career, marriage, children, or a normal life span. In certain embodiments, children and adolescents may have symptoms of post-traumatic stress disorder such as, for example and without limitation, disorganized or agitated behavior, repetitive play that expresses aspects of the trauma, frightening dreams which lack recognizable content, and trauma-specific reenactment. In certain embodiments, a symptom can be stress associated with memory recall.

As used herein, the term “symptom cluster” refers to a set of signs, symptoms, or a set of signs and symptoms, that are grouped together because of their relationship to each other or their simultaneous occurrence. For example, in certain embodiments post-traumatic stress disorder is characterized by three symptom clusters: re-experiencing/intrusion, avoidance/numbing, and hyperarousal.

As used herein, the term “re-experiencing/intrusion” refers to at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders. In certain embodiments, the physiological reactivity manifests in at least one of abnormal respiration, abnormal cardiac rate of rhythm, abnormal blood pressure, abnormal function of a special sense, and abnormal function of sensory organ.

As used herein, the term “avoidance/numbing” refers to at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, and sense of a foreshortened future. Restricted range of effect characterized by diminished or restricted range or intensity of feelings or display of feelings can occur. A sense of a foreshortened future can manifest in thinking that one will not have a career, marriage, children, or a normal life span. Avoidance/numbing can also manifest in social anxiety and anxiety with unfamiliar surroundings.

As used herein, the term “hyperarousal” refers to at least one of difficulty falling or staying asleep, irritability or outbursts of anger, difficulty concentrating, hypervigilance, and exaggerated startle response. Potentially threatening stimuli can cause hyperarousal or anxiety.

As used herein, the term “significantly” refers to a set of observations or occurrences that are too closely correlated to be attributed to chance. For example, in certain embodiments, “significantly changes”, “significantly reduces”, and “significantly increases” refers to alterations or effects that are not likely to be attributed to chance. In certain embodiments, statistical methods can be used to determine whether an observation can be referred to as “significantly” changed, reduced, increased, or altered. Patients diagnosed with post-traumatic stress disorder may feel “on guard”, uneasy, and intensely anxious. Depression, anxiety, panic attacks, and bipolar disorder are often associated with post-traumatic stress disorder. Alcohol and drug abuse are also common. In certain embodiments, disorders cormorbid with post-traumatic stress disorder can include for example but without limitation depression, alcohol abuse, and drug abuse.

As used herein, the term “Clinician-Administered PTSD Scale (CAPS)” refers to a measure for diagnosing and assessing post-traumatic stress syndrome. The CAPS is a 30-item structured interview that corresponds to the DSM-IV criteria for PTSD. Different versions of this measure have been developed.

As used herein, the term “Clinician-Administered PTSD Scale-Part1 (CAPS-1)” is a version of CAPS that assesses current and lifetime PTSD and is also known as CAPS-DX (for diagnosis).

As used herein, the term “Clinician-Administered PTSD Scale-Part 2 (CAPS-2)” refers to a version of CAPS used to assess one week symptom status in patients with post-traumatic stress disorder and also refers to a CAPS-SX (for symptom),

As used herein, the term “Clinician-Administered PTSD Scale for children and adolescents (CAPS-CA)” refers to a version of CAPS developed for children and adolescents.

As used herein, the term “Impact of Event Scale (IES)” refers to a scale developed by Mardi Horowitz, Nancy Wilner, and William Alvarez to measure subjective stress related to a specific event. It is a self-reported assessment and can be used to make measurements over time to monitor a patient's status.

As used herein, the term “Impact of Event Scale-Revised (IES-R)” refers to the revision of the IES developed by Daniel S. Weiss and Charles Marmar to assess the hyperarousal symptom cluster of PTSD.

As used herein, the term “Clinical Global Impression Scale (CGI)” refers to a scale for making psychiatric assessments. Patients are interviewed and the CGI is used to measure the severity of illness (CGI-S), global improvement (CGI-I), and efficacy index.

As used herein, the term “Clinical Global Impression Severity of Illness (CGI-S)” refers to an assessment of the patient's current symptoms. Generally, it is rated on a seven-point scale, ranging from a score of 1 (normal) to 7 (extremely ill). The severity of the patient's illness is compared to the severity of other patients' illness. For example, the CGI-S score can be used to measure a patient's condition after treatment with Compound A, and the scores before and after treatment may be compared.

As used herein, the term “Clinical Global Impression Improvement (CGI-I)” refers to a comparison of a patient's current condition to his baseline condition. Generally, it is rated on a seven-point scale ranging from 1 (very much improved) to 7 (very much worse). The CGI-I score can be used to measure, for example, improvement of post-traumatic stress disorder in response to Compound A treatment.

As used herein, the term “efficacy index” refers to a score taken on CGI and compares the patient's baseline condition with a ratio of current therapeutic benefit to severity of side effects. Generally, it is rated on a four-point scale ranging from 1 (none) to 4 (outweighs therapeutic effect). In assessing post-traumatic stress disorder, the efficacy index could, for example, assess the risk-benefit of treating with a therapy such as Compound A. As used herein, the term “Duke Global Rating for PTSD scale (DGRP)” refers to a scale that measures severity and improvement for each of the three PTSD symptom clusters: re-experiencing/intrusion, avoidance/numbing, and hyperarousal as well as overall PTSD severity.

As used herein, the term “Duke Global Rating for PTSD scale-Improvement (DGRP-I)” refers to a scale used to distinguish responders (DGRP-I of 1 (very much improved) and 2 (much improved)) from nonresponders (DGRP-I>2) of in response to a treatment, for example, Compound A, for post-traumatic stress disorder.

As used herein, the term “Hamilton Anxiety Scale (HAM-A)” refers to a scale developed by Max Hamilton in 1959 to diagnose and quantify symptoms of anxiety and post-traumatic stress disorder. It consists of 14 items, each defined by a series of symptoms. No standardized probe questions to elicit information from patients or behaviorally specific guidelines were developed for determining item scoring. Each item is rated on a 5-point scale, ranging from 0 (not present) to 4 (severe). Items include assessing anxious mood, fears, intellectual effects, somatic complaints, e.g. on musculature, cardiovascular symptoms, tension, insomnia, depressed mood, somatic sensory complaints, respiratory symptoms, gastrointestinal symptoms, autonomic symptoms, genitourinary symptoms, and behavior at the time of assessment. For example, a reduction in the HAM-A score would indicate improvement in a disorder such as post-traumatic stress disorder.

As used herein, the terms “Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI)” refer to additional scales that diagnose, assess, measure a sign, symptom, symptom cluster of post-traumatic stress disorder, anxiety, or depression.

As used herein, the term “score” refers to a score of at least one item or parameter measured on a scale that measures at least one sign, symptom, or symptom cluster of psychiatric symptoms, anxiety, or post-traumatic stress disorder. In certain embodiments, a score measures the frequency, intensity, or severity of a sign, symptom, symptom cluster, associated symptom, or impact on daily life of post-traumatic stress disorder. In certain embodiments, a “score” that assesses post-traumatic stress disorder can be signifcantly changed, for example, by treatment for post-traumatic stress disorder.

As used herein, the term “endpoint score” refers to a score on an instrument that assesses post-traumatic stress disorder taken during or after treatment.

As used herein, the term “baseline score” refers to a score on an instrument that assesses post-traumatic stress disorder prior to initiation of a treatment.

As used herein, the term “overall score” refers to a sum of the scores on an instrument that assesses post-traumatic stress disorder. In certain embodiments, an overall score is the sum of a score of at least one of symptoms, symptoms clusters, associated symptoms, impact on daily life, efficacy, and improvement.

As used herein, the term “relapse” refers to reoccurrence or worsening of at least one symptom of a disease or disorder in a patient.

As used herein the phrase “therapeutically effective amount” refers to the amount sufficient to provide a therapeutic outcome regarding at least one sign, symptom, or associated symptom of a disease, disorder, or condition. For example, the disease, disorder, or condition is PTSD.

As used herein, the phrase “improving resilience” refers to increasing the ability of a patient to experience a traumatic event without suffering post-traumatic stress disorder or with less post-event symptomatology or disruption of normal activities of daily living. In certain embodiments, improving resilience can, in certain embodiments, reduce at one of the signs, symptoms, or symptom clusters of post-traumatic stress disorder.

As used herein, the term “coadministering” refers to a dosage regimen for a first agent that overlaps with the dosage regimen of a second agent, or to simultaneous administration of the first agent and the second agent. A dosage regimen is characterized by dosage amount, frequency, and duration. Two dosage regimens overlap if between initiation of a first and initiation of a second administration of a first agent, the second agent is administered.

As used herein, the term “agent” refers to a substance including, but not limited to a chemical compound, such as a small molecule or a complex organic compound, a protein, such as an antibody or antibody fragment or a protein comprising an antibody fragment, or a genetic construct which acts at the DNA or mRNA level in an organism.

As used herein, the term “dopamine β-hydroxylase activity” refers to conversion of dopamine to norepinephrine mediated by dopamine β hydroxylase. Activity of dopamine β-hydroxylase can be assayed by measuring dopamine or norepinephrine levels.

As used herein, the term “modulates” refers to changing or altering an activity, function, or feature. For example, an agent may modulate levels of a factor by elevating or reducing the levels of the factor.

As used herein, the term catecholamine refers to a compound that contains an amine group attached to a catechol portion and that serves as a hormone or neurotransmitter. By way of example and without limitation, dopamine and norepinephrine are catecholamines.

Provided herein are methods of treating a patient diagnosed with post-traumatic stress disorder. The methods include administering to the patient a therapeutically effective amount of Compound A.

In certain embodiments the methods further comprise coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the at least one other agent is a SSRI selected from paroxetine, sertraline, citalopram, escitalopram, and fluoxetine.

In certain embodiments the at least one other agent is a SNRI selected from duloxetine, mirtazapine, and venlafaxine.

In certain embodiments the at least one other agent is a NRI selected from bupropion and atomoxetine.

In certain embodiments the at least one other agent is disulfuram.

In certain embodiments the at least one other agent is the adenosine A2A receptor antagonist istradefylline.

In certain embodiments the at least one other agent is a sodium channel blocker selected from lamotrigine, carbamazepine, oxcarbazepine, and valproate.

In certain embodiments the at least one other agent is a calcium channel blocker selected from lamotrigine and carbamazepine.

In certain embodiments the at least one other agent is the central and peripheral alpha adrenergic receptor antagonist prazosin.

In certain embodiments the at least one other agent is the central alpha adrenergic agonist clonidine.

In certain embodiments the at least one other agent is the central or peripheral beta adrenergic receptor antagonist propranolol.

In certain embodiments the least one other agent is an atypical antidepressant/antipsychotic selected from olanzepine, risperidone, and quetiapine.

In certain embodiments the least one other agent is a tricyclic selected from amitriptyline, amoxapine, desipramine, doxepin, imipramine, nortriptyline, protiptyline, and trimipramine.

In certain embodiments the least one other agent is an anticonvulsant selected from lamotrigine, carbamazepine, oxcarbazepine, valproate, topiramate, and levetiracetam.

In certain embodiments the least one other agent is the glutamate antagonist topiramate.

In certain embodiments the least one other agent is a GABA agonist selected from valproate and topiramate.

In certain embodiments the least one other agent is the partial D2 agonist aripiprazole.

In certain embodiments the patient has abnormal brain levels of at least one catecholamine.

In certain embodiments the Compound A reduces dopamine β hydroxylase activity in the brain of the patient.

In certain embodiments the Compound A modulates brain levels of at least one catecholamine in the patient.

In certain embodiments the at least one catecholamine is norepinephrine and the Compound A reduces brain levels of the norepinephrine in the patient.

In certain embodiments the at least one catecholamine is dopamine and the Compound A elevates brain levels of the dopamine in the patient.

In certain embodiments the Compound A reduces stress associated with memory recall in the patient.

In certain embodiments the Compound A reduces at least one of the frequency and intensity of at least one sign of the post-traumatic stress disorder in the patient.

In certain embodiments the Compound A reduces at least one of the frequency and intensity of at least one symptom of the post-traumatic stress disorder in the patient.

In certain embodiments the Compound A reduces at least one of the frequency and intensity of at least one symptom cluster of the post-traumatic stress disorder in the patient, wherein the symptom cluster is selected from re-experiencing/intrusion, avoidance/numbing, and hyperarousal.

In certain embodiments the re-experiencing/intrusion comprises at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders.

In certain embodiments the physiological reactivity comprises at least one of abnormal respiration, abnormal cardiac rate of rhythm, abnormal blood pressure, abnormal function of at least one special sense, and abnormal function of at least one sensory organ.

In certain embodiments the at least one special sense is selected from sight, hearing, touch, smell, taste, and sense.

In certain embodiments the at least one sensory organ is selected from eye, ear, skin, nose, tongue, and pharynx.

In certain embodiments the avoidance/numbing comprises at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, and anxiety associated with unfamiliar surroundings.

In certain embodiments the hyperarousal comprises at least one of difficulty falling or staying asleep, irritability or outbursts of anger, difficulty concentrating, hypervigilance, exaggerated startle response, and anxiety from potentially threatening stimuli.

In certain embodiments the Compound A does not reduce the physical ability of the patient to respond appropriately and promptly to the potentially threatening stimuli.

In certain embodiments the Compound A reduces the difficulty of staying asleep by reducing stress associated with memory recall and dreaming.

In certain embodiments the patient is a child or an adolescent.

In certain embodiments the Compound A reduces at least one of the frequency and intensity of at least one sign or symptom of the post-traumatic stress disorder in the patient, wherein the sign or symptom is selected from disorganized or agitated behavior, repetitive play that expresses aspects of the trauma, frightening dreams which lack recognizable content, and trauma-specific reenactment.

In certain embodiments the Compound A reduces the incidence of at least one disorder comorbid with post-traumatic stress disorder selected from drug abuse, alcohol abuse, and depression in the patient.

In certain embodiments the Compound A is administered to the patient once or twice a day.

In certain embodiments the Compound A does not cause at least one of drowsiness, lassitude, or alteration of mental and physical capabilities.

In certain embodiments the Compound A is administered to the patient before or immediately after a traumatic event.

In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

In certain embodiments the Compound A significantly changes a score on at least one of CAPS, CAPS-2, CAPS-CA, IES, IES-R, CGI, CGI-S, CGI-I, DGRP, DGRP-I, HAM-A, SI-PTSD, PTSD-I, PSS-I, MADRS, BDI, HAM-D, RHRSD, MDI, GDS-30, and CDI.

In certain embodiments the Compound A significantly reduces an endpoint score compared to a baseline score on at least one of CAPS, CAPS-2, IES, IES-R, and HAMA.

In certain embodiments the Compound A significantly increases the proportion of responders on the CGI-I having a CGI-I score of at least one of 1 (very much improved) and 2 (much improved).

In certain embodiments the Compound A increases the proportion of responders on the DGRP-I having a DGRP-I score of at least one of 1 (very much improved) and 2 (much improved).

In certain embodiments an overall score of at least 65 on at least one of the CAPS and the CAP-2 is indicative of post-traumatic stress disorder.

In certain embodiments an overall score of at least 18 on HAM-A is indicative of anxiety disorder.

In certain embodiments a score of at least 3 on at least one of the CGI-I and the DGRP-I is indicative of post-traumatic stress disorder.

Also provided are methods of treating post-traumatic stress disorder in a patient. The methods include diagnosing the patient with post-traumatic stress disorder; administering to the patient a therapeutically effective amount of Compound A; assessing at least one of sign, symptom, and symptom cluster of post-traumatic stress disorder; and determining that the post-traumatic stress syndrome is improved if the Compound A reduces at least one of sign, symptom, and symptom cluster of post-traumatic stress disorder.

In certain embodiments the method includes coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the Compound A reduces at least one of the frequency and intensity of at least one sign of the post-traumatic stress disorder in the patient.

In certain embodiments the Compound A reduces at least one of the frequency and intensity of at least one symptom of the post-traumatic stress disorder in the patient.

In certain embodiments the Compound A reduces at least one of the frequency and intensity of at least one symptom cluster of the post-traumatic stress disorder in the patient, wherein the symptom cluster is selected from re-experiencing/intrusion, avoidance/numbing, and hyperarousal.

In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of improving resilience in a patient. The methods include administering a therapeutically effective amount of Compound A.

In certain embodiments the method includes coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the Compound A reduces at least one of the frequency and intensity of at least one sign of the post-traumatic stress disorder in the patient.

In certain embodiments the Compound A reduces at least one of the frequency and intensity of at least one symptom of the post-traumatic stress disorder in the patient.

In certain embodiments the Compound A reduces at least one of the frequency and intensity of at least one symptom cluster of the post-traumatic stress disorder in the patient, wherein the symptom cluster is selected from re-experiencing/intrusion, avoidance/numbing, and hyperarousal.

In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of diagnosing post-traumatic stress disorder in a patient. The methods include administering to the patient a therapeutically effective amount of Compound A and assessing at least one of sign, symptom, or symptom cluster of post-traumatic stress disorder; and diagnosing post-traumatic stress disorder in the patient if the Compound A reduces at least one of sign, symptom, and symptom cluster of post-traumatic stress disorder.

In certain embodiments the patient is a child, adolescent, or adult.

Various scales can assess post-traumatic stress disorder (PTSD) and the effect of rufinamde and other therapies on the treatment and prevention of the disorder. These are, for example and without limitation, Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD

Interview (PTSD-I), PTSD Symptom Scale (PSS-I), Mini International Neuropsychiatric Interview (MINI), Montgomery-Asberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI). These measures generally are assessed by interviews or questionnaires. In certain embodiments, not all the parts of a scale are administered. In certain embodiments, the scales are used for diagnosing and assessing signs, symptoms, associated symptoms, or impact on daily life of PTSD. In certain embodiments, one or more scales are used to diagnose, assess, or confirm post-traumatic stress disorder in a patient. In certain embodiments, scales will measure signs, symptoms, symptom clusters by scoring at least one of the frequency and intensity of the signs, symptoms, or symptom clusters.

Examples of scales for post-traumatic stress disorder assessment are versions of CAPS, including CAPS, CAPS-1, and CAPS-2, which score 17 core PTSD symptoms with these items:

1. Recurrent and intrusive trauma recollections

2. Distress when exposed to trauma reminders

3. Acting or feeling as if event were recurring

4. Recurrent and distressing dreams of event

5. Efforts to avoid thoughts or feelings

6. Efforts to avoid activities or situations

7. Inability to recall trauma or trauma aspects

8. Markedly diminished interest in significant activities

9. Feelings of detachment or estrangement from others

10. Restricted range of affect

11. Sense of a foreshortened future

12. Difficulty falling or staying asleep

13. Irritability or outbursts of anger

14. Difficulty concentrating

15. Hypervigilance

16. Exaggerated startle response

17. Physiologic reactivity

Questions also target the impact of symptoms on social and occupational functioning or daily life, improvement in symptoms since a previous CAPS administration, overall response validity, overall PTSD severity, and frequency and intensity of associated symptoms. These items are:

18. Impact on Social Functioning

19. Impact on Occupational Functioning

20. Global Improvement (since earlier measurement occasion)

21. Rating Validity

22. Global Improvement

23. Guilt over acts committed or omitted

24. Survivor Guilt

25. Homicidality

26. Disillusionment with authority

27. Feelings of hopelessness

28. Memory Impairment

29. Sadness and depression

30. Feelings of being overwhelmed

To assess the frequency of symptoms, interviewers follow standard questions, clarifying or rephrasing as needed. Standard questions, by way of example and without limitation, are: Have you ever had unwanted memories of the traumatic event? What were they like? What did you remember? If the question requires rephrasing, the interviewer can ask a question such as: Did they ever occur while you were awake or only in dreams? or How often have you had these memories in the past month (week)? A score of 0 indicates a frequency of never, 1 indicates once or twice, 2 indicates once or twice a week, 3 indicates several times a week, and 4 indicates daily of almost every day.

To assess the intensity of symptoms, an interviewer may ask standard questions such as by way of example and without limitation: How much distress or discomfort did these memories cause you? Were you able to put them out of your mind and think about something else? How hard did you have to try? How much did they interfere with your life? A score of 0 indicates none, 1 indicates mild, minimal distress or disruption of activities, 2 indicates moderate, distress clearly present but still manageable, some disruption of activities, 3 indicates severe, considerable distress, difficulty dismissing memories, marked disruption of activities, and 4 indicates extreme, incapacitating distress, cannot dismiss memories, unable to continue activities.

In certain embodiments the scoring rule used counts a symptom as present if it has a frequency of at least 1 and an intensity of at least 2. In certain embodiments severity scores are calculated by summing the frequency and intensity ratings for each symptom.

In certain embodiments, a total or overall score of all items on a version of CAPS is calculated. In certain embodiments, a total score for each symptom cluster is calculated. In certain embodiments, a total score for core symptoms of PTSD is calculated. In certain embodiments, an endpoint score is compared to a baseline score to determine the change in severity of post-traumatic stress disorder. In certain embodiments, a significant reduction of an endpoint score compared to a baseline score is considered improvement of PTSD. In certain embodiments, an overall score on CAPS, CAPS-1, CAPS-2, or CAPS-CA greater than 65 is indicative of PTSD.

Another example is the IES which assesses 15 items: 7 items measure intrusive symptoms and 8 items measure avoidance symptoms. The self assessed items ask how frequently each of the following comments are true: I thought about it when I didn't mean to, I avoided letting myself get upset when I thought about it or was reminded of it, I tried to remove it from memory, I had trouble falling asleep or staying asleep because of pictures or thoughts about it that came into my mind, I had waves of strong feelings about it, I had dreams about it, I stayed away from reminders of it, It felt as it hadn't happened or wasn't real, I tried not to talk about it, Pictures about it popped into my mind, Others things kept making me think about it, I was aware that I still had a lot of feelings about it, but I didn't deal with them, I tried not to think about it, Any reminder brought back feelings about it, and My feelings were kind of numb. The items are generally rated on a four point scale: 0 (not at all), 1 (rarely), 3 (sometimes), and 5 (often). The total of the scores provide an overall assessment of the severity of the symptoms or overall subjective stress. It has been suggested that a score from 0 to 8 is in the subclinical range, 9-25 is in the mild range, 26-43 is in the moderate range, and greater than 44 is in the severe range of stress.

In certain embodiments, a total or overall score of all items on IES is calculated. In certain embodiments, a total score for each symptom cluster is calculated. In certain embodiments, an endpoint score is compared to a baseline score to determine the change in severity of PTSD. In certain embodiments, a reduction of an endpoint score by 30% compared to a baseline score is considered improvement of PTSD.

The IES-R, a revision of the IES, changed the IES by splitting the original IES item, I had trouble falling asleep or staying asleep into two items: I had trouble falling asleep and I had trouble staying asleep and by adding six items to the IES items. These additional items are: I felt irritable and angry, I was jumpy and easily startled, I found myself acting or feeling as though I was back at that time, I had trouble concentrating, Reminders of it caused me to have physical reactions, such as sweating, trouble breathing, nausea, or a pounding heart, and I felt watchful or on guard. The scoring system also changed to 0 (not at all), 1 (a little bit), 2 (moderately), 3 (quite a bit), and 4 (extremely).

In certain embodiments, a total or overall score of all items on IES-R is calculated. In certain embodiments, a total score for each symptom cluster is calculated. In certain embodiments, an endpoint score is compared to a baseline score to determine the change in severity of post-traumatic stress disorder. In certain embodiments, a significant reduction of an endpoint score compared to a baseline score on the IES-R is considered improvement of post-traumatic stress disorder.

In the DGRP-I scale, the effectiveness of Compound A in treating post-traumatic stress disorder can be assessed by measuring the increase in the proportion of responders on the DGRP-I having a DGRP-I of 1 (very much improved) or 2 (much improved). In certain embodiments, a score of at least 3 on the DGRP-I is indicative of post-traumatic stress

In the CGI, the effectiveness of Compound A to treat post-traumatic stress disorder can be assessed by the CGI-S, CGI-I, and efficacy index. For example, in certain embodiments, an increase in the proportion of responders on the CGI-I having a CGI-I of 1 (very much improved) or 2 (much improved) after treatment indicates that the treatment is effective. In certain embodiments, a score of at least 3 on the CGI-I is indicative of post-traumatic stress disorder. In certain embodiments, the efficacy index on the CGI can measure the efficacy of Compound A for treatment of post-traumatic stress disorder.

In HAMA-A, to assess anxiety or post-traumatic stress disorder, generally a total or overall score of all items on HAM-A is calculated. In certain embodiments, an endpoint score is compared to a baseline score on HAM-A to determine the change in severity of anxiety and post-traumatic stress disorder. In certain embodiments, a significant reduction of an endpoint score compared to a baseline score on HAM-A is considered improvement of anxiety and post-traumatic stress disorder. In certain embodiments, an overall score on HAM-A of at least 18 is indicative of anxiety and post-traumatic stress disorder.

In general, Compound A or a pharmaceutically acceptable derivative will be administered in therapeutically effective amounts, either singly or in combination with another therapeutic agent. The pharmaceutical compositions will be useful, for example, for the treatment of post-traumatic stress disorder.

Pharmaceutically acceptable derivatives include acids, bases, enol ethers, and esters, esters, hydrates, solvates, and prodrug forms. The derivative is selected such that its pharmokinetic properties are superior with respect to at least one characteristic to the corresponding neutral agent. The Compound A may be derivatized prior to formulation.

A therapeutically effective amount of Compound A or a pharmaceutically acceptable derivative may vary widely depending on the severity of the post-traumatic stress disorder, the age and relative health of the subject, the potency of the compound used and other factors. In certain embodiments a therapeutically effective amount is from about 0.1 milligram per kg (mg/kg) body weight per day to about 50 mg/kg body weight per day. In other embodiments the amount is about 1.0 to about 10 mg/kg/day. Therefore, In certain embodiments a therapeutically effective amount for a 70 kg human is from about 7.0 to about 3500 mg/day, while in other embodiments it is about 70 to about 700 mg/day.

One of ordinary skill in the art of treating such diseases will be able to ascertain a therapeutically effective amount of Compound A for post-traumatic stress disorder without undue experimentation and in reliance upon personal knowledge and the disclosure of this application. In general, by way of example and without limitation, Compound A will be administered as pharmaceutical compositions by one of the following routes: oral, systemic (e.g., transdermal, intranasal or by suppository) or parenteral (e.g., intramuscular, intravenous or subcutaneous). Compositions can, by way of example and without limitation, take the form of tablets, pills, capsules, semisolids, powders, sustained release formulations, solutions, suspensions, elixirs, aerosols, or any other appropriate composition and are comprised of, in general, Compound A in combination with at least one pharmaceutically acceptable excipient. Acceptable excipients are, by way of example and without limitation, non-toxic, aid administration, and do not adversely affect the therapeutic benefit of the compound. Such excipient may be, for example, any solid, liquid, semisolid or, in the case of an aerosol composition, gaseous excipient that is generally available to one of skill in the art.

Solid pharmaceutical excipients include by way of example and without limitation starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from for example and without limitation water, ethanol, glycerol, propylene glycol and various oils, including those of petroleum, animal, vegetable or synthetic origin (e.g., peanut oil, soybean oil, mineral oil, sesame oil, etc.). Preferred liquid carriers, particularly for injectable solutions, include by way of example and without limitation water, saline, aqueous dextrose and glycols. Compressed gases may be used to disperse the compound in aerosol form. Inert gases suitable for this purpose are by way of example and without limitation nitrogen, carbon dioxide, nitrous oxide, etc.

The pharmaceutical preparations can by way of example and without limitation, moreover, contain preservatives, solubilizers, stabilizers, wetting agents, emulsifiers, sweeteners, colorants, flavorants, salts for varying the osmotic pressure, buffers, masking agents or antioxidants. In certain embodiments, they can contain still other therapeutically valuable substances. Other suitable pharmaceutical carriers and their formulations are described in A. R. Alfonso Remington's Pharmaceutical Sciences 1985, 17th ed. Easton, Pa.: Mack Publishing Company.

The amount of Compound A in the composition may vary widely depending for example, upon the type of formulation, size of a unit dosage, kind of excipients and other factors known to those of skill in the art of pharmaceutical sciences. In general, the final composition will comprise from 10% w to 90% w of the compound, preferably 25% w to 75% w, with the remainder being the excipient or excipients. Preferably the pharmaceutical composition is administered in a single unit dosage form for continuous treatment or in a single unit dosage form ad libitum when relief of symptoms is specifically required.

The Compound A or a pharmaceutically acceptable derivative thereof is administered simultaneously with, prior to, or after administration of one or more of the above agents.

The invention is further illustrated by the following non-limiting examples.

### EXAMPLES

### Example 1

A clinical study is performed to demonstrate the efficacy and tolerability of Compound A in the treatment of post-traumatic stress disorder (PTSD).

The research design includes an 8-week randomized, double-blind, placebo-controlled treatment trial of Compound A for the treatment of PTSD. After signing an informed consent and meeting inclusion/exclusion criteria, patients are randomized to receive either Compound A or placebo for the 8-week duration. During the study a pharmacist maintains the randomization log and verify the order for the placebo or Compound A in look-a-like tablets. Patients' symptoms, side effects and compliance is assessed bi-weekly.

Based on symptomatology and occurrence of side effects, the investigator may increase the medication in 20-40 mg increments, as tolerated, until a maximum therapeutic benefit is achieved. The dosing is once per day unless twice per day is better tolerated. Compliance is assessed by pill count at week 4 and week 8.

Patients is given supportive clinical management during the clinic visits. An investigator is available by telephone 24 hrs a day in case of emergency. Patients may be seen more often if needed.

Efficacy is measured by the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)

The subject inclusion criteria are:
Diagnosis of PTSD that is confirmed by Mini International Neuropsychiatric Interview (MINI) and CAPS
Age 13 or older
No substance abuse or dependence for the previous 4 weeks (except for nicotine and caffeine)
Free of psychotropic medication for 2 weeks (except 4 weeks for fluoxetine)
Clinically normal physical and laboratory examination (Liver function tests (LFTs) up to 2.5 times the normal limit is allowed.)
Women of childbearing potential must be using medically approved methods of birth control such as a condom, birth control pill, Depo-Provera, or diaphragm with spermicides
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to Compound A
Unstable general medical conditions
Score ≦6 on Question #10 of MADRS regarding suicidal ideation
Women who are pregnant, planning to become pregnant or breastfeed during the study

Fulfillment of only one exit criterion is needed to exit the study. Exit criteria are:
Completion of the study
Severe and intolerable side effects to Compound A or placebo treatment
Acute development of suicidal ideation, homicidal ideation or psychotic symptoms
Worsening of symptoms as measured by a score of 7 (very much worse) on CGI-I
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 2

A clinical study is performed to demonstrate the efficacy and tolerability of Compound A in the prevention of PTSD.

The research design includes an open-ended randomized, double-blind, placebo-controlled treatment trial of Compound A for the prevention of PTSD. After signing an informed consent and meeting inclusion/exclusion criteria, patients are randomized to receive either Compound A versus placebo for the 8-week duration. During the study a pharmacist maintains the randomization log and verify the order for the placebo or Compound A in look-a-like tablets. Patients' symptoms, side effects and compliance are assessed bi-weekly.

Based on symptomatology and occurrence of side effects, the investigator can increase the medication in 20-40 mg increments, as tolerated, until a maximum therapeutic benefit is achieved. The dosing is once per day unless twice per day is better tolerated. Compliance is assessed by pill count at week 4 and week 8.

Patients are given supportive clinical management during the clinic visits. An investigator is available by telephone 24 hrs a day in case of emergency. Patients may be seen more often if needed.

Efficacy is measured by the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)
Diagnostic and Statistical Manual IV (DSM-IV)

The subject inclusion criteria are:
Absence of PTSD, confirmed by MINI and CAPS
Age 13 or older
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The exclusion criteria are:
History of PTSD
Lifetime history of bipolar I, psychotic, or cognitive disorders
Actively suicidal, homicidal, or psychotic
History of sensitivity to Compound A
Unstable general medical conditions
Score ≧6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to Compound A or placebo treatment
Acute development of suicidal ideation, homicidal ideation or psychotic symptoms
Appearance of signs or symptoms compatible with a diagnosis of PTSD.
Participant's explicit request to exit the study
The need for additional psychotropic drugs, other than the study drug or adjunctive medication as specified in the protocol, for the control of the subjects psychiatric symptoms.
The subject becomes pregnant during the course of the study.
Investigator's judgment that it is no longer in the best interest of the patient to continue in the study.

### Example 3

A clinical study is conducted to demonstrate the efficacy and tolerability of Compound A combination therapy in the treatment of PTSD.

The research design includes an 8-week randomized, double-blind, placebo-controlled treatment trial of Compound A for the treatment of PTSD. After signing an informed consent and meeting inclusion/exclusion criteria, the patient is randomized to receive either Compound A or placebo for 8-week duration. Patients can also receive therapeutically effective doses of prazosin, valproate, carbamazepine, or topiramate in combination with Compound A or placebo.

During the study a pharmacist maintains the randomization log and verifies the order for the placebo or Compound A in look-a-like tablets. Patients' symptoms, side effects and compliance is assessed bi-weekly. Based on symptomatology and occurrence of side effects, the investigator increases the medication in 20-40 mg increments, as tolerated, until a maximum therapeutic benefit is achieved. The dosing is once per day unless twice per day is better tolerated. Compliance is assessed by pill count at week 4 and week 8.

Patients are given supportive clinical management during the clinic visits. An investigator is available by telephone 24 hrs a day in case of emergency. Patients may be seen more often if needed.

Efficacy is measured by the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)

The subject inclusion criteria are:
Diagnosis of PTSD, confirmed by MINI and CAPS
Age 13 or older
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to Compound A
Unstable general medical conditions
Score ≧6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to Compound A or placebo treatment
Acute development of suicidal ideation, homicidal ideation or psychotic symptoms
Symptoms worsen as measured by a Score of 7 (very much worse) on CGI-I
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 4

A clinical study is performed to demonstrate the efficacy and tolerability of Compound A in the treatment of PTSD in children.

The research design includes an 8-week randomized, double-blind, placebo-controlled treatment trial of Compound A for the treatment of PTSD.

After signing an informed consent and meeting inclusion/exclusion criteria, patients are randomized to receive either Compound A or placebo for an 8-week duration. During the study a pharmacist maintains the randomization log and verify the order for the placebo or Compound A in look-a-like tablets. Patients' symptoms, side effects and compliance are assessed bi-weekly.

Based on symptomatology and occurrence of side effects, the investigator can increase the medication in 20-40 mg increments, as tolerated, until a maximum therapeutic benefit is achieved. The dosing is once per day unless twice per day is better tolerated. Compliance is assessed by pill count at week 4 and week 8.

Patients are given supportive clinical management during the clinic visits. An investigator is available by telephone 24 hrs a day in case of emergency. Patients may be seen more often if needed.

Efficacy is measured by the following assessment scales:
Global Assessment of Functioning (GAF)
Clinician Administered PTSD Scale (CAPS)
Clinician Administered PTSD Scale (CAPS-CA)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)

The subject inclusion criteria are:
Diagnosis of PTSD, confirmed by MINI and CAPS
Age 12 or younger
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to Compound A
Unstable general medical conditions
Score ≧6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to Compound A or placebo treatment
Acute development of suicidal ideation, homicidal ideation or psychotic symptoms
Symptoms worsen as measured by a Score of 7 (very much worse) on CGI-1.
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 5

Bovine and human dopamine-β-hydroxylase activity was assayed by measuring the conversion of tyramine to octopamine. Bovine adrenal dopamine-β-hydroxylase was obtained from Sigma Chemicals (St Louis, Mo., USA) whereas human dopamine-β-hydroxylase was purified from the culture medium of the neuroblastoma cell line SK-N-SH. The assay was performed at pH 5.2 and 32° C. in a medium containing 0.125 M NaAc, 10 mM fumarate, 0.5-2 μM CuSO4, 0.1 mg.ml⁻¹ catalase, 0.1 mM tyramine and 4 mM ascorbate. In a typical assay, 0.5-1 milliunits of enzyme were added to the reaction mixture and, subsequently, a substrate mixture containing catalase, tyramine and ascorbate was added to initiate the reaction (final volume of 200 μl). Samples were incubated with or without the appropriate concentration of nepicastat (S-enantiomer) or (R)-5-Aminomethyl-1-(5,7-difluoro-1,2,3,4-tetrahydronaphth-2-yl)-2,3-dihydro-2-thioxo-1H-imidazole hydrochloride (R-enantiomer) at 37° C. for 30-40 minutes. The reaction was quenched by the stop solution containing 25 mM EDTA and 240 μM 3-hydroxytyramine (internal standard). The samples were analysed for octopamine by reverse phase high pressure liquid chromatography (HPLC) using ultraviolet-detection at 280 nM. The HPLC chromatography run was carried out at the flow rate of 1 ml.min⁻¹ using a LiChroCART 125-4 RP-18 column and isocratic elution with 10 mM acidic acid, 10 mM 1-heptane sulfonic acid, 12 mM tetrabutyl ammonium phosphate and 10% methanol. The remaining percent activity was calculated based on controls, corrected using internal standards and fitted to a non-linear four-parameter concentration-response curve.

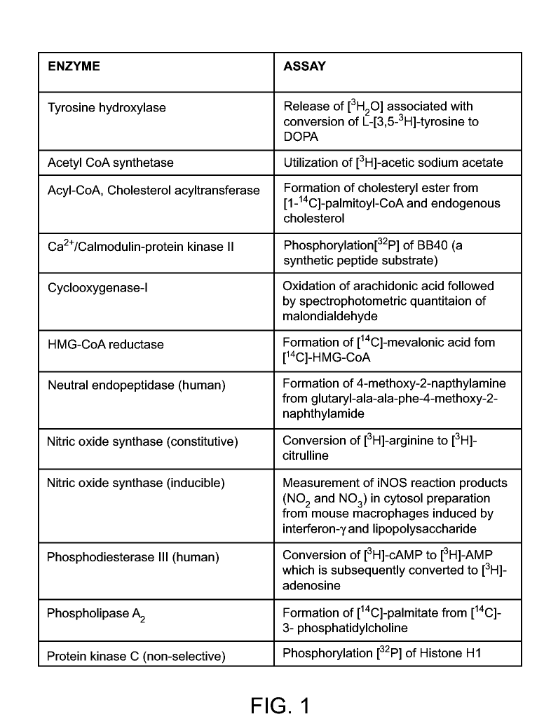

The activity of nepicastat at twelve selected enzymes and receptors was determined using established assays. Details of individual receptor radioligand binding assays can be found in Wong et al (1993). A brief account of the principle underlying each of the enzymatic assays is given in FIG. 1. Binding data were analyzed by iterative curve-fitting to a four parameter logistic equation. Ki values were calculated from IC₅₀ values using the Cheng-Prusoff equation. Enzyme inhibitory activity was expressed as IC₅₀ (concentration required to produce 50% inhibition of enzyme activity). Male SHRs (15-16 weeks old, Charles River, Wilmington, Mass., USA) were used in the study. On the day of the study, animals were weighed and randomly assigned to be dosed with either vehicle (control) or the appropriate dose of nepicastat (3, 10, 30 or 100 mg.kg⁻¹, po) or (R)-5-Aminomethyl-1-(5,7-difluoro-1,2,3,4-tetrahydronaphth-2-yl)-2,3-dihydro-2-thioxo-1H-imidazole hydrochloride (30 mg.kg⁻¹, po) three consecutive times, twelve hours apart. At six hours after the third dose, the rats were anaesthetized with halothane, decapitated and tissues (cerebral cortex, mesenteric artery and left ventricle) were rapidly harvested, weighed, placed in iced perchloric acid (0.4 M), frozen in liquid nitrogen and stored at −70° C. until subsequent analysis. To quantify noradrenaline and dopamine concentrations, tissues were homogenized by brief sonication and centrifuged at 13,000 rpm for 30 minutes at 4° C. The supernatant, spiked with 3,4-dihydroxybenzylamine (internal standard), was assayed for noradrenaline and dopamine by HPLC using electrochemical detection.

Male beagle dogs (10-16 kg, Marshall Farms USA Inc, North Rose, N.Y., USA) were used in the study. On the day of the study, dogs were weighed and randomly assigned to be orally dosed with either empty capsules (control) or the appropriate dose of nepicastat (0.05, 0.5, 1.5 or 5 mg.kg⁻¹; po, b.i.d.) for 5 days. At six hours following the first dose on day-5, the dogs were euthanized with pentobarbital and the tissues (cerebral cortex, renal artery, left ventricle) were rapidly harvested. The tissues were subsequently processed and analysed for noradrenaline and dopamine as described above.

Male beagle dogs were randomized to be orally dosed with either empty capsules (control) or nepicastat (2 mg.kg⁻¹, po, b.i.d.) for 15 days. Daily venous blood samples were drawn, six hours after the first dose, for measurement of plasma concentrations of dopamine and noradrenaline. The samples were collected in tubes containing heparin and glutathione, centrifuged at −4° C. and the separated plasma was stored at −70° C. until analysis.

Nepicastat ((S)-5-aminomethyl-1-(5,7-difluoro-1,2,3,4-tetrahydronaphth-2-yl)-1,3-dihydroimidazole-2-thione hydrochloride) and the corresponding R-enantiomer ((R)-5-Aminomethyl-1-(5,7-difluoro-1,2,3,4-tetrahydronaphth-2-yl)-2,3-dihydro-2-thioxo-1H-imidazole hydrochloride) were synthesized. In studies involving SHRs, the drugs were dissolved in distilled water and dosed orally with a gavage needle. In the dog studies, the drugs were filled in capsules and dosed orally. All doses are expressed as free base equivalents.

All data are expressed as mean±s.e.mean. Tissue and plasma catecholamine data were analysed using a non-parametric one-way analysis of variance (ANOVA) or two-way ANOVA, respectively, followed by pairwise comparison using Fisher LSD test. P<0.05 was considered statistically significant.

Nepicastat (S-enantiomer) and (R)-5-Aminomethyl-1-(5,7-difluoro-1,2,3,4-tetrahydronaphth-2-yl)-2,3-dihydro-2-thioxo-1H-imidazole hydrochloride (R-enantiomer) produced concentration-dependent inhibition of bovine and human dopamine-β-hydroxylase activity. The calculated IC₅₀'s for nepicastat were 8.5±0.8 nM and 9.0±0.8 nM for the bovine and human enzyme, respectively. (R)-5-Aminomethyl-1-(5,7-difluoro-1,2,3,4-tetrahydronaphth-2-yl)-2,3-dihydro-2-thioxo-1H-imidazole hydrochloride was slightly less potent (IC₅₀'s of 25.1±0.6 nM and 18.3±0.6 nM for the bovine and human enzyme, respectively) than nepicastat.

Nepicastat had negligible affinity (IC₅₀s or Ki's>10 μM) for a range of other enzymes (tyrosine hydroxylase, acetyl CoA synthetase, acyl CoA-cholesterol acyl transferase, Ca²⁺/calmodulin protein kinase II, cyclooxygenase-I, HMG-CoA reductase, neutral endopeptidase, nitric oxide synthase, phosphodiesterase III, phospholipase A₂, and protein kinase C) and neurotransmitter receptors (α_{1A}, ⊕_{1B}, α_{2A}, α_{2B}, β₁ and β₂ adrenoceptors, M₁ muscarinic receptors, D₁ and D₂ dopamine receptors, μ opioid receptors, 5-HT_{1A}, 5-HT_{2A}, and 5-HT_{2C} serotonin receptors).

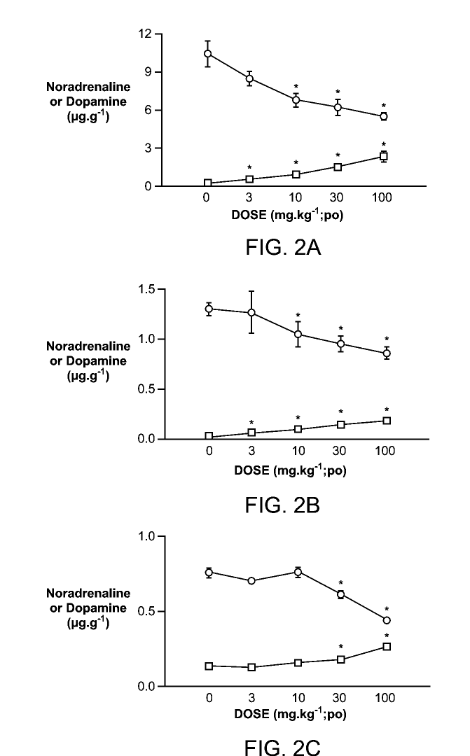

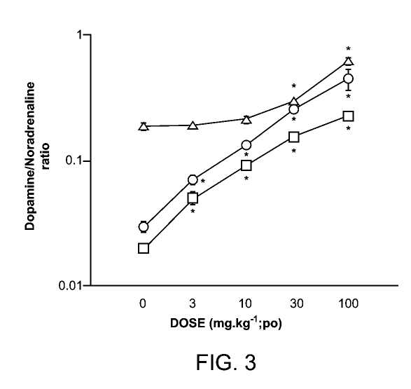

Basal tissue catecholamine content (μg.g⁻¹ wet weight) in control animals were as follows: mesenteric artery (noradrenaline, 10.40±1.03; dopamine, 0.25±0.02), left ventricle (noradrenaline, 1.30±0.06; dopamine, 0.02±0.00) and cerebral cortex (noradrenaline, 0.76±0.03; dopamine, 0.14±0.01). Nepicastat produced dose-dependent reduction in noradrenaline content and enhancement of dopamine content and dopamine/noradrenaline ratio in the three tissues which were studied (FIGS. 2 & 3). FIG. 2 shows the effects of nepicastat on tissue noradrenaline (O) and dopamine (□) content in the mesenteric artery (A), left ventricle (B) and cerebral cortex (C) of SHRs. Data are expressed as mean±s.e.mean; n=7-9 per group. * p<0.05 vs control (O). FIG. 3 shows the effects of nepicastat on tissue dopamine/noradrenaline ratio in the mesenteric artery (O), left ventricle (□) and cerebral cortex (A) of SHRs. Data are expressed as mean±s.e.mean, n=7-9 per group. * p<0.05 vs control (O).

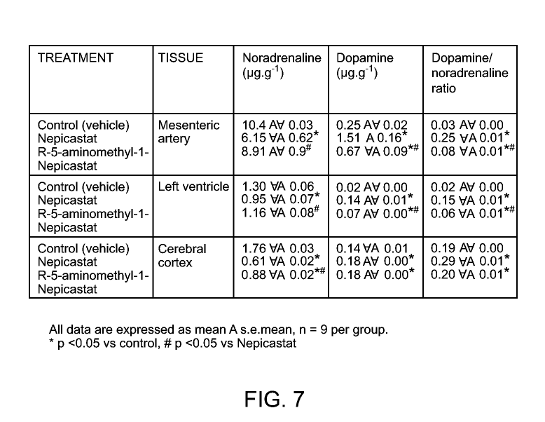

These changes attained statistical significance (p<0.05) at doses of >3 mg.kg⁻¹ in the mesenteric artery and left ventricle but only at doses of 30 and 100 mg.kg⁻¹ in the cerebral cortex. At the highest dose studied (100 mg.kg⁻¹, po), the decreases in noradrenaline were 47%, 35%, 42% and increases in dopamine were 820%, 800% and 86% in the mesenteric artery, left ventricle and cerebral cortex, respectively. When tested at 30 mg.kg⁻¹, po, the S-enantiomer (nepicastat) produced significantly greater changes in catecholamine content, as compared to the R-enantiomer ((R)-5-Aminomethyl-1-(5,7-difluoro-1,2,3,4-tetrahydronaphth-2-yl)-2,3-dihydro-2-thioxo-1H-imidazole hydrochloride), in the mesenteric artery and left ventricle (FIG. 7). FIG. 7 shows the effect of nepicastat and (R)-5-Aminomethyl-1-(5,7-difluoro-1,2,3,4-tetrahydronaphth-2-yl)-2,3-dihydro-2-thioxo-1H-imidazole hydrochloride, at 30 mg.kg⁻¹; po, on noradrenaline content, dopamine content and dopamine/noradrenaline ratio in mesenteric artery, left ventricle and cerebral cortex of SHRs. Data are expressed as mean+/−sem. n=9 per group. * p<0.05 vs. control, #p<0.05 vs nepicastat.

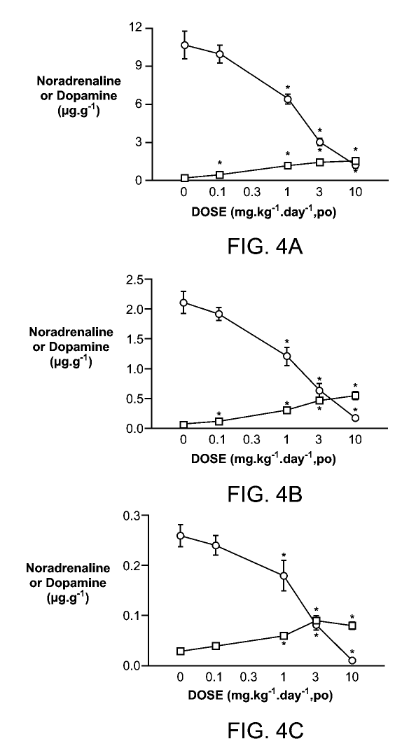

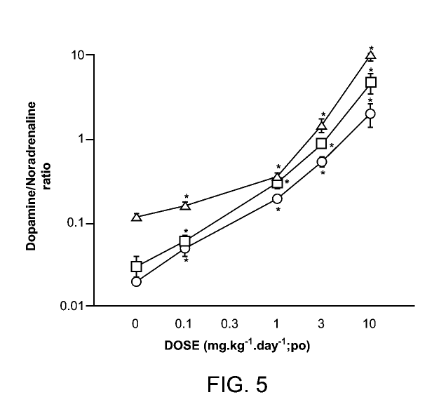

Basal tissue catecholamine content (μg.g⁻¹ wet weight) in control animals were as follows: renal artery (noradrenaline, 10.7±1.05; dopamine, 0.22±0.01), left ventricle (noradrenaline, 2.11±0.18; dopamine, 0.07±0.03) and cerebral cortex (noradrenaline, 0.26±0.02; dopamine, 0.03±0.00). When compared to control animals, nepicastat produced dose-dependent reduction in noradrenaline content and enhancement of dopamine content and dopamine/noradrenaline ratio in the three tissues which were studied (FIGS. 4 & 5). FIG. 4 shows the effects of nepicastat on tissue noradrenaline (O) and dopamine (□) content in renal artery (A), left ventricle (B) and cerebral cortex (C) of beagle dogs. Data are expressed as mean±s.e.mean; n=8 per group. * p<0.05 vs control (O). FIG. 5 shows the effects of nepicastat on tissue dopamine/noradrenaline ratio in the renal artery (O), left ventricle (□) and cerebral cortex (Δ) of beagle dogs. Data are expressed as mean±s.e.mean, n=8 per group. * p<0.05 vs control (O).

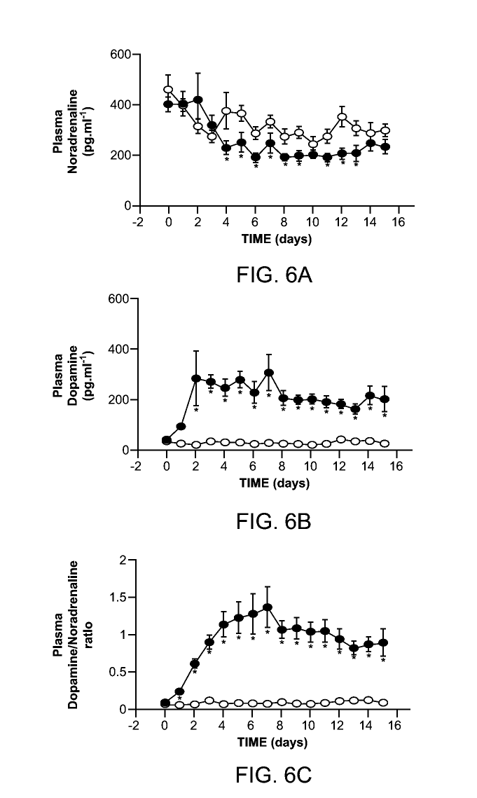

These changes attained statistical significance (p<0.05) at doses of >0.1 mg.kg⁻¹.day⁻¹ in the three tissues. At the highest dose studied (5 mg.kg⁻¹, b.i.d., po), the decreases in noradrenaline were 88%, 91% and 96% and increases in dopamine were 627%, 700% and 166% in the renal artery, left ventricle and cerebral cortex, respectively. Baseline concentrations of catecholamines in two groups of animals were not significantly different from each other: plasma noradrenaline and dopamine concentrations were 460.3±59.6 and 34.4±11.9 pg.ml⁻¹, respectively, in the control group and 401.9±25.5 and 41.1±8.8 pg.ml⁻¹, respectively, in the nepicastat-treated group. When compared to the control group, nepicastat (2 mg.kg⁻¹, b.i.d, po) produced significant decreases in plasma concentrations of noradrenaline and increases in plasma concentrations of dopamine and dopamine/noradrenaline ratio (FIG. 6). FIG. 6 shows the Effects of nepicastat on plasma concentrations of noradrenaline (A), dopamine (B) and dopamine/noradrenaline ratio (C) in beagle dogs. Control dogs (O); nepicastat-treated dogs (•). Data are expressed as mean±s.e.mean; n=8 per group. * p<0.05 vs control. The peak reduction (52%) in plasma concentration of noradrenaline was observed on day-6 of dosing whereas the peak increase (646%) in plasma concentration of dopamine was observed on day-7 of dosing.

Inhibitory modulation of sympathetic nerve function, through pharmacological means, is an attractive therapeutic strategy for the management of congestive heart failure, inasmuch as elevated activity of this system has been implicated in the progressive worsening of the disease. The aim of this study was to pharmacologically characterize the effects of nepicastat, a compound which modulates noradrenaline synthesis in sympathetic nerves by inhibiting the enzyme dopamine-β-hydroxylase.

nepicastat was shown to be a potent inhibitor of human and bovine dopamine-β-hydroxylase in vitro. The inhibitory effects of the compound were stereospecific since the S-enantiomer (nepicastat) was marginally, but significantly, more potent than the R-enantiomer ((R)-5-Aminomethyl-1-(5,7-difluoro-1,2,3,4-tetrahydronaphth-2-yl)-2,3-dihydro-2-thioxo-1H-imidazole hydrochloride). nepicastat displayed a high degree of selectivity for dopamine-β-hydroxylase as the compound possessed negligible affinity for twelve other enzymes and thirteen neurotransmitter receptors.

Inhibition of dopamine-β-hydroxylase in vivo would be expected to result in elevated levels of the substrate (dopamine) and diminished levels of the product (noradrenaline) in tissues which receive noradrenergic innervation. This expectation was borne out in experiments which investigated the effects of nepicastat on catecholamine levels in central and peripheral tissues in vivo. In both SHRs and beagle dogs, nepicastat produced dose-dependent reductions in noradrenaline content and increases in dopamine content in peripheral (mesenteric or renal artery, left ventricle) and central (cerebral cortex) tissues. In this respect, (R)-5-Aminomethyl-1-(5,7-difluoro-1,2,3,4-tetrahydronaphth-2-yl)-2,3-dihydro-2-thioxo-1H-imidazole hydrochloride was less potent than nepicastat which is consistent with the lower IC₅₀ of the former enantiomer for the enzyme. Although dopamine/noradrenaline ratio was also elevated, there did not appear to be stoichiometric replacement of noradrenaline with dopamine. The most likely explanation for this finding is that tissue levels of dopamine may have been underestimated due to intraneuronal metabolism of dopamine.

The ability of nepicastat to alter catecholamine levels in the cerebral cortex suggests that the drug does penetrate the blood brain barrier. In dogs, the magnitude of the changes in catecholamines in the cerebral cortex appeared comparable to those in peripheral tissues. In SHRs, however, nepicastat, at low doses (≦10 mg.kg⁻¹), produced significant changes in noradrenaline and dopamine content in peripheral tissues without affecting catecholamines in the cerebral cortex. This suggests that, at least in SHRs, the drug does possess modest peripheral selectivity. We have also shown that nepicastat attenuates sympathetically mediated cardiovascular responses and lowers blood pressure in SHRs without affecting motor activity (Hegde et al., 1996 a & b); these findings will be reported in a separate manuscript.

Plasma noradrenaline concentrations provide a useful measure of overall sympathetic nerve activity although this parameter may be influenced by alterations in neuronal uptake and metabolic clearance of the catecholamine. Baseline concentrations of noradrenaline in the plasma were surprisingly elevated in the dogs and is, perhaps, a reflection of the initial stress induced by the phlebotomy blood-sampling procedure. Nevertheless, compared to the control group, nepicastat produced significant decreases in plasma noradrenaline concentrations consistent with reduced transmitter synthesis and release although an indirect effect, secondary to facilitation of neuronal uptake or metabolic clearence, cannot be discounted. Since released noradrenaline represents a small fraction of the total neuronal noradrenaline stores, an inhibitior of noradrenaline biosynthesis would affect noradrenaline release only after existing stores of the catecholamine have been sufficiently depleted. Accordingly, the decreases in plasma noradrenaline concentrations did not attain statistical significance until 4 days of dosing with nepicastat suggesting gradual modulation of the sympathetic nervous system. It should be recognized that measurements of plasma noradrenaline concentrations alone do not account for regional differences in noradrenaline release (Esler et al., 1984), which underscores the need for making measurements of organ-specific noradrenaline spillover rates in future studies.

A growing body of evidence suggests that chronic activation of the sympathetic nervous system in congestive heart failure is a maladaptive response. This contention is supported by clinical trials which have shown a beneficial effect of carvedilol in congestive heart failure patients with respect to long-term morbidity and mortality. However, it should be noted that most patients do require some level of sympathetic drive to support cardiovascular homeostasis. Indeed, the therapeutic value of β-blockers, including carvedilol, may be limited by their propensity to cause hemodynamic deterioration especially during initiation of therapy. This unwanted effect, which results from abrupt withdrawal of sympathetic support, necessitates careful dose-titration. Inhibitors of dopamine-β-hydroxylase, such as nepicastat, may be devoid of this undesirable effect for the following reasons. First, this class of drugs would attenuate, but not abolish, noradrenaline release and, second, they produce gradual modulation of the system thereby obviating the need for dose-titration. Another advantage of nepicastat over β-blockers is that it enhances dopamine levels which, via agonism of dopamine receptors, may have salutary effects on renal function such as renal vasodilation, diuresis and natriuresis.

In summary, nepicastat is a potent, selective and orally active inhibitor of dopamine-β-hydroxylase which may be of value in the treatment of cardiovascular disorders associated with over-activation of the sympathetic nervous system.

### Example 6

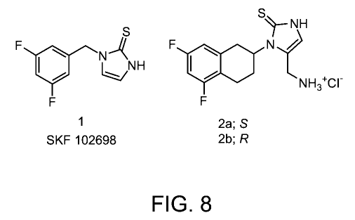

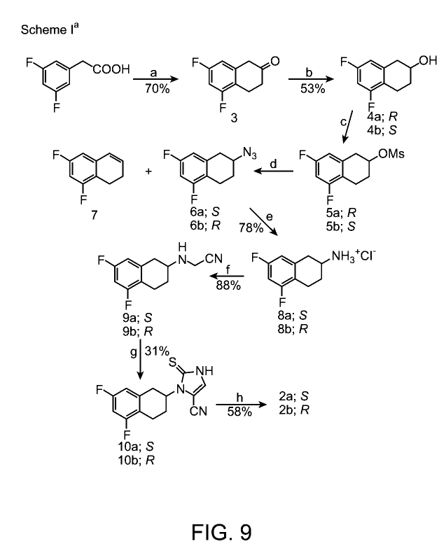

Synthesis of nepicastat (2a) (FIG. 8 and FIG. 9). Oral administration of 2a to spontaneously hypertensive rats (SHR) and normal dogs produced potent and dose-dependent increases in tissue dopamine (DA)/norepinephrine (NE) ratios in peripheral arteries (renal or mesenteric), left ventricle and cerebral cortex. Chronic oral administration of 2a to normal dogs also produced sustained increases in the plasma DA/NE ratio. In conscious SHR, acute oral administration of 2a produced dose-dependent and long-lasting (>4 h) antihypertensive effects and also attenuation of the pressor responses to pre-ganglionic sympathetic nerve stimulation. Serum T₃ and T₄ levels were unaffected by a dose (6.2 mg/kg, po, b.i.d. for 10 days) which elevated the dopamine/norepinephrine ratio in the mesenteric artery. On the basis of its ability to potently modulate the sympathetic drive to cardiovascular tissues, 2a is currently in clinical evaluation for the treatment of congestive heart failure.

Congestive heart failure (CHF) is a leading cause of mortality in the United States. CHF is characterized by marked activation of the sympathetic nervous system (SNS) and renin-angiotensin system (RAS). The simultaneous activation of these two neurohormonal systems has been increasingly implicated in the perpetuation and progression of CHF. Therapeutic interventions which block the effects of these neurohormonal systems are likely to favorably alter the natural history of CHF. Indeed, angiotensin-converting enzyme (ACE) inhibitors, which block formation of angiotensin II, have been shown to reduce morbidity and mortality in CHF patients. ACE inhibitors, however, have a limited indirect ability to attenuate the SNS. Inhibition of the SNS with β-adrenoceptor antagonists is a promising approach that is currently under clinical evaluation. An alternative strategy to directly modulate the SNS is inhibition of norepinephrine (NE) biosynthesis via inhibition of dopamine β-hydroxylase (DBH), the enzyme responsible for conversion of NE to dopamine (DA). Inhibition of DBH would be expected to reduce tissue levels of NE and elevate tissue levels of DA thereby increasing the tissue DA/NE ratio. This approach has potential advantages over β-adrenoceptor antagonists, such as reduced stimulation of a-adrenoceptors and elevated DA levels that can produce renal vasodilation, natriuresis and diminished aldosterone release. Previous DBH inhibitors, such as fusaric acid and SKF 102698, have drawbacks such as low potency and specificity, that have precluded their clinical development in heart failure.

This example shows 2a (nepicastat) to be a potent and selective inhibitor of DBH related to SKF 10269. The preparation of 2a (Scheme I) was based upon the chiral reduction of tetralone 3 (available from the AlCl₃-catalyzed Friedel-Crafts reaction of 3,5-difluorophenylacetyl chloride with ethylene in CH₂Cl₂ at −65° C.) under the conditions described by Terashima⁷ (LAH, (−)-1R,2S-N-methylephedrine, 2-ethylaminopyridine) to give R-(+)-tetralol 4a (92-95% ee). Conversion of 4a to the R-(+)-mesylate 5a, followed by reaction with sodium azide afforded a mixture (9:1) of azide 6a and dihydronaphthalene 7. The azide was hydrogenated and the product treated with anhydrous HCl to give S-(−)-amine hydrochloride 8a, converted by a Strecker reaction (formaldehyde bisulfite complex and KCN) to S-(−)-aminonitrile 9a. Formation of the heterocycle 10a was accomplished by sequential diformylation of aminonitrile 9a followed by subsequent treatment with thiocyanic acid. Competing hydrolysis of the nitrile afforded comparable amounts of the primary amide 11a. Reduction of nitrile 10a to amine 2a (93-96% ee) was accomplished using LAH in THF. The enantiomer 2b (91.6% ee) was available by the same above described route using (+)-1S,2R-N-methylephedrine as a chiral auxiliary in the Terashima reduction of ketone 3. The absolute configuration of the chiral center in 4a,b, and thus 2a,b was based upon literature precedence of the previously described S-(−)-2-tetralol.

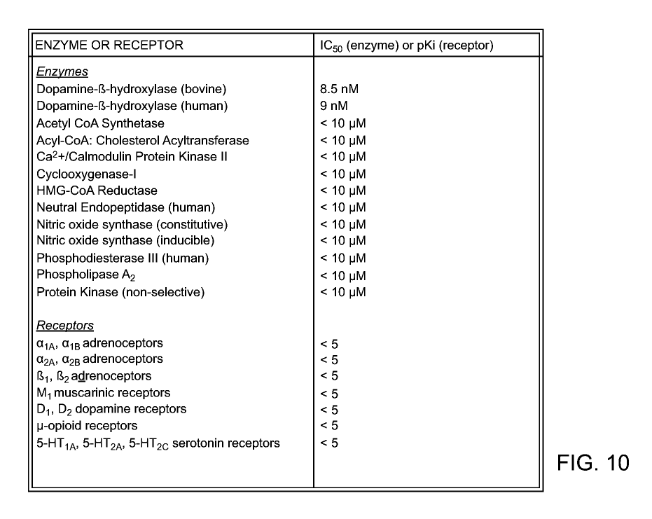

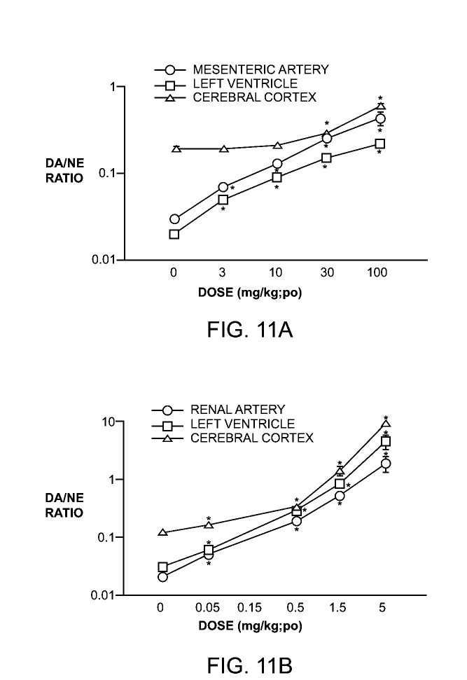

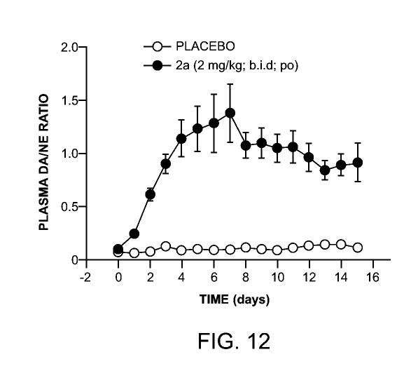

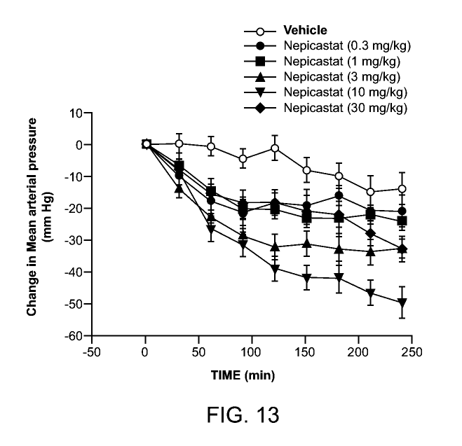

Tetralin 2a was demonstrated to be a competitive inhibitor of bovine (IC₅₀=8.5±0.8 nM) and human (IC₅₀=9.0±0.8 nM) DBH. The R-enantiomer 2b (IC₅₀S=25.1±0.6 nM; 18.3±0.6 nM) and 1 (IC₅₀s=67.0±4.2 nM; 85.0±3.7 nM) are less potent inhibitors of the bovine and human enzymes, respectively. Compound 2a showed weak affinity for a range of other enzymes and neurotransmitter receptors (FIG. 10). These data suggest that 2a is a potent and highly selective inhibitor of DBH in vitro. Moreover, the S-enantiomer is approximately 2-3 fold more potent than the R-enantiomer suggesting stereoselectivity. The in vivo biochemical effects of 2a, 2b and 1 were evaluated in spontaneously hypertensive rats (SHR) and normal beagle dogs. Oral administration of 2a produced dose-dependent increases in DA/NE ratios in the artery (mesenteric or renal), left ventricle and cerebral cortex in SHR (FIG. 11A) and dogs (FIG. 11B). At the highest dose tested (100 mg/kg in SHR and 5 mg/kg in dogs) the maximal increases in DA/NE ratio were 14, 11 and 3.2 fold (in SHR) and 95, 151 and 80 fold (in dogs) in the artery, left ventricle and cerebral cortex, respectively. When tested at 30 mg/kg in SHR, SKF 102698 (1) increased the DA/NE ratio by 5.5-fold, 3.5-fold and 2.7-fold, whereas 2a, at the same dose, increased the ratio by 8.3, 7.5 and 1.5 fold in the mesenteric artery, left ventricle and cerebral cortex, respectively. The R-enantiomer 2b, at 30 mg/kg in SHR, produced only 2.6, 3.5 and 1.1 fold increases in the DA/NE ratio in the mesenteric artery, left ventricle and cerebral cortex, respectively. These data suggest that 2a produces the expected biochemical effects in both SHR and dogs but is more potent in the latter species. Furthermore, 2a is more potent than its R-enantiomer 2b and SKF 102698 (1) in SHR. The chronic effects of 2a (14.5 day treatment) on the plasma DA/NE ratio were investigated in normal dogs. Oral administration of 2a (2 mg/kg; b.i.d) produced a significant increase in the DA/NE ratio that attained its peak effect at approximately 6-7 days, then plateaued to a new steady-state between 7-14 days (FIG. 12). The in vivo hemodynamic activity of 2a was further assessed in conscious, restrained SHR, a model having high sympathetic drive to cardiovascular tissues. Oral dosing of 2a resulted in a dose-dependent antihypertensive effect (FIG. 13). A maximal decrease in mean blood pressure of 53±4 mmHg (33% reduction relative to vehicle control) was observed at the 10 mg/kg dose. The response was slow in onset, reaching its plateau in 3-4 h. The precise reason for the loss of anti-hypertensive efficacy at the highest dose (30 mg/kg) is unclear at present. Heart rate was not significantly affected except for a slight yet significant decrease at 10 and 30 mg/kg, (9.8 and 10.5%, respectively). Following this study, the rats were pithed and the effects of 2a on the pressor response to pre-ganglionic nerve stimulation (PNS) of the spinal cord were evaluated 5 h after dosing. The frequency-pressor response curve was shifted significantly (p<0.05) to the right in a dose-dependent manner (maximum shift of 5 fold in the frequency-response curve). The heart rate response to PNS was not significantly affected. These data suggest that 2a inhibits the sympathetic drive to the vasculature and is the probable mechanism for its anti-hypertensive effect in SHR.

Since the heterocyclic portion of 2a is structurally similar to methimazole, a known potent suppressor of mammalian thyroid function, the effects of 2a on thyroid function were evaluated at doses of 2.0 and 6.2 mg/kg, po, b.i.d in iodine-deficient Sprague-Dawley rats (n=9-12) for 10 days. Methimazole (1 mg/kg, po, b.i.d.), used as a positive control, caused a significant reduction in serum levels of T₃ (day 3, 31%, p<0.05; days 7 and 9, 42% and 44%, p<0.01) and T₄ (days 3 and 7, 46% and 58%, p<0.01) 4 h post-dose, whereas 2a showed no significant effects throughout the study (days 3, 7 and 9). Both doses of 2a significantly raised the DA/NE ratio in the mesenteric artery (p<0.01 relative to vehicle controls) but not in the cortex 4 h after the final dose on day 10.

The findings of this study suggest that 2a (nepicastat) is a potent, selective and orally active inhibitor of DBH. The compound is also devoid of significant behavioral effects in animal models and these findings will be the subject of a future publication. As compound 2a (nepicastat) effectively modulates the sympathetic drive to cardiovascular tissues, it is currently undergoing development for the treatment of CHF.

FIG. 9 shows ^{a}(a) i: SOCl₂; ii: AlCl₃, CH₂Cl₂, ethylene, −65° C.; (b) (−)-1R,2S-N-methylephedrine, 2-ethylaminopyridine, 1M LAH in Et₂O, <−60° C. for the R-enantiomer or (+)-1S,2R-N-methylephedrine, 2-ethylaminopyridine, 1M LAH in Et₂O, <−60° C. for the S-enantiomer; (c) MsCl, Et₂O, Et₃N, −15° C.; (d) NaN₃, DMSO, 50° C.; (e) i: H₂, 10% Pd/C, EtOAc, 60 psi; ii: 1M HCl/Et₂O; (f) NaOH, formaldehyde-sodium bisulfite complex, KCN, H₂O, 50-80° C.; (g) i: n-butyl formate, 120° C.; ii: t-BuOK, ethyl formate, THF, −15° C.; iii: 1M HCl, EtOH, KSCN; (h) i: 1M LAH in THF, 20° C.; ii: HCl/Et₂O, MeOH.

FIG. 10 shows a table describing the interaction of nepicastat at DBH and a range of selected enzymes and receptors.

FIG. 11: (A)—Effects of 2a on tissue DA/NE ratio in spontaneously hypertensive rats. Animals were dosed orally, 12 h apart, and the tissues were harvested 6 h after the third dose. * p<0.05 vs placebo (vehicle).

(B)—Effects of 2a on tissue DA/NE ratio in normal beagle dogs. Animals were dosed orally, twice a day, for 4.5 days and the tissues were harvested 6 h after the first dose on day 5. * p<0.05 vs placebo (empty capsule).

DA and NE concentrations were assayed by HPLC with electrochemical detection. All data are expressed as mean±standard error of mean. n=9 per group.

FIG. 12: Effects of chronic administration of 2a on plasma DA/NE ratio in normal beagle dogs. Animals were dosed orally, b.i.d., for 14.5 d. Blood sampling was done on each day 6 h after the first dose. 2a produced significant (p<0.05) increases in DA/NE ratio at all time-points compared to the placebo group. DA and NE concentrations in plasma were assayed by HPLC with electrochemical detection. All data are expressed as mean±standard error of mean. n=8 per group.

FIG. 13: Effects of orally administered 2a on mean arterial pressure in conscious, restrained spontaneously hypertensive rats (SHR). SHR were lightly anesthetized with ether and instrumented for measurement of arterial pressure and drug administration. The animals were placed in restrainers and allowed to recover for 30-40 minutes. After obtaining baseline measurements, the animals were treated, orally, with either vehicle or the appropriate dose of 2a and hemodynamic parameters were continously recorded for 4 h. 2a produced significant (p<0.05) lowering of mean artarial pressure at all doses and time points, except at 0.3 mg/kg (180, 210 and 240 min) and 1 mg/kg (30, 210 and 240 min). All data are expressed as mean±standard error of mean. n=6-8 per group. Melting points were determined on a Uni-Melt Thomas Hoover Capillary Melting Point Apparatus or a Mettler FB 81HT cell with a Mettler FP90 processor and are uncorrected. Mass spectra were obtained with either a Finnigan MAT 8230 (for electron-impact or chemical ionization) or Finnigan MAT TSQ70 (for LSIMS) spectrometer. ¹H NMR spectra were recorded on a Bruker ACF300, AM300, AMX300 or EM390 spectrometer and chemical shifts are given in ppm (δ) from tetramethylsilane as internal standard. IR spectra were recorded on a Nicolet SPC FT-IR spectrometer. UV spectra were recorded on a Varian Cary 3 UV-Visible spectrometer, Leeman Labs Inc. Optical rotations were measured in a Perkin-Elmer Model 141 polarimeter. Chiral HPLC measurements were performed on a Regis Chiral AGP column (4.6×100 mm) eluting with 2% acetonitrile-98% 20 mM KH₂PO₄ (pH 4.7) at 1 mL/min at 20° C.

5,7-Difluoro-2-tetralone (3). SOCl₂ (100 mL) was added in one portion to 3,5-difluorophenylacetic acid (100 g, 0.58 mol) and after stirring for 15 h, the volatiles were evaporated under reduced pressure. The resulting oily acid chloride was dissolved in CH₂Cl₂ (200 mL) and added dropwise to a mechanically stirred suspension of AlCl₃ (154 g, 1.16 mol) in CH₂Cl₂ (1.0 L). The stirred suspension was cooled to an internal temperature of −65° C. in a dry ice/acetone bath, and the acid chloride solution was added at such a rate in order to maintain an internal temperature <−60° C. After the addition was complete, ethylene gas was bubbled through the reaction mixture at a rapid rate for 10 min at −65° C. The reaction mixture was allowed to warm to 0° C. over 2 h with stirring, and was then cooled to −10° C. and treated with H₂O (500 mL) initially dropwise, followed by rapid addition. The organic layer was separated, washed with brine (100 mL) and dried over MgSO₄. Evaporation under reduced pressure gave a dark oily residue which was distilled in vacuo on a Kugelrohr collecting material boiling between 90-110° C. (1.0 to 0.7 mm Hg). The distillate was redistilled at 100-105° C. (0.3 mm Hg) to give 3 as a white solid, (73.6 g, 0.40 mol; 70%): mp 46° C.; IR (KBr) 1705 cm⁻¹; ¹H NMR (CDCl₃) δ 2.55 (t, J=7.5 Hz, 2H), 3.10 (t, J=7.5 Hz, 2H), 3.58 (s, 2H), 6.70 (m, 2H); MS m/z 182 (M⁺). Anal. Calcd for C₁₀H₈F₂O: C, 65.93; H, 4.42. Found: C, 65.54; H, 4.42.

(R)-(+)-2-Hydroxy-5,7-difluoro-1,2,3,4-tetrahydronaphthalene (4a). A solution of (−)-1R,2S-N-methylephedrine (81.3 g, 0.454 mol) in anhydrous Et₂O (1.1 L) was added dropwise (45 min) to 1.0 M lithium aluminum hydride (416 mL, 0.416 mol) in Et₂O at a rate sufficient to maintain a gentle reflux. After the addition was complete, the reaction mixture was heated at reflux for 1 h then allowed to cool to room temperature. A solution of 2-ethylaminopyridine (111 g, 0.98 mole) in anhydrous Et₂O (100 mL) was added (45 min) at such a rate as to maintain a gentle reflux. The reaction mixture was heated at reflux for a further 1 h, during which time a light yellow-green suspension appeared. The mixture was cooled to an internal temperature of −65° C. using a dry ice-acetone bath and a solution of 5,7-difluoro-2-tetralone (23.0 g, 126 mmol) in Et₂O (125 mL) was added dropwise at a rate maintaining the internal temperature below −60° C. After the addition was complete, the mixture was stirred at −65° C. to −68° C. for 3 h and quenched by the addition of MeOH (100 mL) maintaining the internal temperature below −60° C. The reaction was stirred for a further 10 min at −65° C. and allowed to warm to approximately −20° C. A solution of 3N HCl (2 L) was then added at a rate to limit the temperature to <35° C. After stirring at an increased rate to achieve total dissolution, the layers were separated and the ethereal layer was washed with brine (200 mL) and dried (MgSO₄). The ethereal solution was evaporated under reduced pressure and the residue dissolved in warm Et₂O (20 mL) followed by the addition of hexane (200 mL). The seeded solution was cooled in an ice bath and maintained at 0° C. for 1 h whereupon the resulting deposited crystals were collected and dried in vacuo to give alcohol 4a (10.9 g, 47%): mp 85° C.; [α]²⁵_{D} +38.1° (c=1.83, CHCl₃); 93.4% ee by chiral HPLC: ¹H NMR (CDCl₃) δ 1.70 (br s, 1H), 1.76-1.88 (m, 2H), 1.99-2.06 (m, 2H), 2.63-3.08 (m, 3H), 4.15 (m, 1H), 6.60 (m, 2H). Anal. Calcd for C₁₀H₁₀F₂O: C, 65.21; H, 5.47. Found: C, 65.38: H, 5.42. The spectra for the (S)-enantiomer 4b are identical: mp 84-85° C.; [α]²⁵_{D} −37.8° (c=1.24, CHCl₃); 92.4% ee by chiral HPLC. Anal. Calcd for C₁₀H₁₀F₂O: C, 65.21; H, 5.47. Found: C, 65.47; H, 5.39.

(R)-(+)-2-Methanesulfonyloxy-5,7-difluoro-1,2,3,4-tetrahydronaphthalene (5a). A solution of R-(+)-5,7-difluoro-2-tetralol 4a (59.0 g, 320 mmol) and Et₃N (74.2 mL, 53.9 g, 530 mmol) in anhydrous Et₂O (1.78 L) was cooled (−15° C.) using an ice-MeOH bath and treated under argon with stirring with MsCl (37.2 mL, 55.3 g, 480 mmol) over 5-10 min. After 5 h the reaction was complete (as determined by TLC) and water was added to dissolve the solids. A small amount of EtOAc was added to help complete dissolution of the solids. The organic phase was separated and washed sequentially with 1N HCl, aq. NaHCO₃, brine and dried over MgSO₄. Evaporation of the solvent gave an off-white solid 5a (87.1 g, 332 mmol), used directly in the next step. Trituration of a small sample with i-Pr₂O gave an analytical sample: mp 78.8-80.5° C.; [α]²⁵_{D} +16.8° (c=1.86, CHCl₃); ¹H NMR δ 2.13-2.28 (m, 2H), 2.78-2.96 (m, 2H), 3.07 (s, 3H), 3.09 (dd, J=17.1 Hz, 4.7, 1H), 3.20 (dd, J=17.2, 4.7 Hz, 1H), 5.20 (m, 1H), 6.67 (m, 2H). Anal. Calcd for C₁₁H₁₂F₂O₃S: C, 50.37; H, 4.61. Found: C, 50.41; H, 4.64. The spectra for the (S)-enantiomer 5b are identical: mp 79.9-80.9° C.; [α]²⁵_{D} −16.6° (c=2.23, CHCl₃). Anal. Calcd for C₁₁H₁₂F₂O₃S: C, 50.37; H, 4.61. Found: C, 50.41; H, 4.65.

(S)-(−)-2-Amino-5,7-difluoro-1,2,3,4-tetrahydronaphthalene hydrochloride (8a). Sodium azide (40.0 g, 0.62 mol) was added to DMSO (1 L) with stirring until a clear solution was obtained. The mesylate 5a (138 g, 0.53 mol) was added in one portion and the mixture heated at 50° C. for 16 h under a N₂ atmosphere. The reaction mixture was diluted with H₂O (1.8 L) and extracted with pentane (4×250 mL) followed by sequentially washing the combined pentane extracts with H₂O (2×100 mL), brine (100 mL) and drying over MgSO₄. Evaporation of the solvent under reduced pressure gave a volatile oil which was rapidly chromatographed on silica using pentane as the eluent to give dihydronaphthalene 7 (8.50 g, 51.2 mmol) as a volatile oil. Further elution with pentane/CH₂Cl₂ (9:1) afforded the azide 6a (101 g, 483 mmol) as a colorless oil: IR (CHCl₃) 2103 cm⁻¹; m/z 171 (M⁺). The azide 6a was dissolved in EtOAc (1200 mL) and hydrogenated over 10% Pd/C (6 g) in a 2.5 L Parr bottle (60 psi) for 6 h. After each hour, the bottle was evacuated and recharged with hydrogen to remove evolved N₂. The resulting mixture was filtered through Celite, stirred with ethereal HCl (1N, 500 mL), and the fine precipitate filtered off and washed with EtOAc, and then anhydrous ether. (The filtration took about 4 h). The moist solid was transferred to a round-bottom flask, and the remaining solvent removed in vacuo to give a white solid 3 (90.4 g, 412 mmol; 77.9%): mp>280° C.; [α]²⁵_{D} −60.2° (c=2.68, MeOH); ¹H NMR (d₆-DMSO) δ 1.79 (m, 1H), 2.33 (m, 1H), 2.63 (m, 1H), 2.83-2.92 (m. 2H), 3.14 (dd, J=16.7, 5.0 Hz, 1H), 3.46 (m, 1H), 6.93 (d, J=9.4 Hz, 1H), 7.00 (dt, J=9.4, 2.5 Hz, 1H). Anal. Calcd for C₁₀H₁₂ClF₂N: C, 54.68; H, 5.51; N, 6.37. Found: C, 54.31; H, 5.52; N, 6.44. The spectra for the (R)-enantiomer 8b are identical: mp>280° C.; [α]²⁵_{D} +58.5° (c=1.63, MeOH). Anal. Calcd for C₁₀H₁₂ClF₂N: C, 54.68; H, 5.51; N, 6.37. Found: C, 54.64; H, 5.51; N, 6.40.

(S)-(−)-(5,7-Difluoro-1,2,3,4-tetrahydronaphth-2-yl)(cyanomethyl)amine (9a). The amine hydrochloride 8a (50.27 g, 229 mmol) was treated with a solution of NaOH (10.0 g. 250 mmol) in water (150 mL), followed by a few additional pellets of NaOH sufficient to obtain a solution. Further water (300 mL) was added and the mixture placed in a 50° C. bath and treated with formaldehyde sodium bisulfite complex (30.8 g, 230 mmol). After the mixture had been stirred for 30 min, KCN (15.0 g, 230 mmol) was added. The reaction mixture was stirred for a further 1 h at 80° C., cooled to room temperature, and extracted with EtOAc to give an oil (51.3 g) which solidified. TLC (5% MeOH—CH₂Cl₂) showed ca. 10-15% of starting amine remained. Chromatography on silica gave 9a (39.4 g) and starting free amine (7.12 g), which quickly forms the carbonate in air. Recycling this amine gave an additional 5.35 g of product. Combined yield (44.8 g, 202 mmol; 87.5%): mp 73.1-76.5° C.; [α]²⁵_{D} −58.0° (c=1.63, CHCl₃); ¹H NMR (CDCl₃) δ 1.50 (br s, 1H), 1.70 (m, 1H), 2.05 (m, 1H), 2.55-3.04 (m, 4H), 3.22 (m, 1H), 3.70 (s, 2H), 6.62 (m, 2H); MS m/z 222 (M⁺). Anal. Calcd for C₁₂H₁₂F₂N₂: C, 64.85; H, 5.44; N, 12.60. Found: C, 65.07; H, 5.47; N, 12.44. The spectra for the (R)-enantiomer 9b are identical: mp 64.4-73.6° C.; [α]²⁵_{D} +52.3° (c=2.12, CHCl₃). Anal. Calcd for C₁₂H₁₂F₂N₂: C, 64.85; H, 5.44; N, 12.60. Found: C, 65.14; H, 5.54; N, 12.53.

(S)-(−)-1-(5,7-Difluoro-1,2,3,4-tetrahydronaphth-2-yl)-5-cyano-2,3-dihydro-2-thioxo-1H-imidazole (10a). The nitrile 9a (44.7 g, 201 mmol) in butyl formate (240 mL) was heated at reflux (120° C. bath) under N₂ for 19 h, and the solvent then removed under reduced pressure. Toluene was added and evaporated to remove last traces of solvent, and the residue was dried under high vacuum to give an oil (53.2 g). The resulting formamide and ethyl formate (48.7 mL, 44.7 g, 604 mmol) in anhydrous THF (935 mL) were cooled in ice/MeOH (−15° C.) and stirred while t-BuOK (1M in THF, 302 mL, 302 mmol) was added over 20 min. After the reaction had been stirred for 18 h, the solvent was evaporated, the residue dissolved in 1N HCl (990 mL) and ethanol (497 mL), and treated with KSCN (78.1 g, 804 mmol). The mixture was stirred for 135 min at 85° C. and then placed in an ice bath to give a precipitate. The filtered solid was loaded as a slurry in 10% MeOH/CH₂Cl₂ on to a silica (1 kg) column packed in hexane. Elution with 10% acetone/CH₂Cl₂ gave 10a (18.05 g, 62.1 mmol; 30.8%): m.p. 240.7-249.2° C.; [∀]²⁵_{D} −69.1° (c=1.18, DMSO); ¹H NMR (d₆-DMSO) δ 2.18 (br m, 1H), 2.47 (m, 1H), 2.75 (m, 1H), 3.03-3.35 (m, 3H), 5.19 (m, 1H), 6.94 (d, J=9.3 Hz, 1H), 7.03 (dt, J=9.3, 2.4 Hz, 1H), 8.29 (s, 1H), 13.3 (br s, 1H); MS m/z 291 (M⁺). Anal. Calcd for C₁₄H₁₁F₂N₃S: C, 57.72; H, 3.80; N, 14.42. Found: C, 57.82; H, 3.92; N, 14.37. (Further elution of the column with 1:1 MeOH/CH₂Cl₂ gave the primary amide 11a: mp 261.9-262.7° C.; [∀]²⁵_{D} −90.5° (c=0.398); IR (KBr) 1593, 1630 cm⁻¹; ¹H NMR (d₆-DMSO) δ 2.14 (m, 1H), 2.15-2.28 (m, 1H), 2.74-3.05 (m, 4H), 5.64 (m, 1H), 6.90 (d, J=9.2 Hz, 1H), 7.05 (dt. J=9.5, 2.4 Hz, 1H), 8.73 (s, 1H), 9.70 (br s, 1H), 13.7 (br s, 1H); MS m/z 309 (M⁺). Anal. Calcd for C₁₄H₁₃F₂N₃OS.0.25H₂O: C, 53.57; H, 4.33; N, 13.39. Found: C, 53.32; H, 3.96: N, 13.24. The spectra for the (R)-enantiomer 10b are identical: mp 243.1-244.7° C.; [∀]²⁵_{D} +74.9° (c=2.14, DMSO). Anal. Calcd for C₁₄H₁₁F₂N₃S: C, 57.72; H, 3.80; N, 14.42. Found: C, 57.85; H, 3.85; N, 14.45.

(S)-1-(5,7-Difluoro-1,2,3,4-tetrahydronaphth-2-yl)-5-aminomethyl-2,3-dihydro-2-thioxo-1H-imidazole. The above nitrile (5.00 g, 17.2 mmol) in THF (75 mL) was stirred under argon in an ice bath until a homogeneous solution was obtained. A solution of LAH in THF (1 M, 34.3 mL, 34.3 mmol) was added dropwise over 10 min, then the solution was stirred for 30 min at 0° C. and allowed to come to room temperature for 1.5 h. The reaction was again cooled to 0° C. and treated with a saturated solution of sodium potassium tartrate until the mixture became freely stirrable. Further tartrate solution (30 mL) was added, followed by 10% MeOH/CH₂Cl₂ (200 mL) and the mixture stirred for 15 min and treated with water (100-150 mL). The organic layer was separated and the aqueous phase extracted with 10% MeOH/CH₂Cl₂ (2×125 mL). The combined extracts were washed, dried (MgSO₄), and evaporated. Chromatography of the residue (5.2 g) on silica eluting with 5% MeOH/CH₂Cl₂ gave 2a as the free amine (2.92 g, 9.89 mmol; 58%): mp 170° C.; [V]²⁵_{D} −11.0° (c=1.59, DMSO). Anal. Calcd for C₁₄H₁₅F₂N₃S.0.25H₂O: C, 56.07; H, 5.21; N, 14.01. Found: C, 56.11; H, 5.10; N, 14.14.

(S)-1-(5,7-Difluoro-1,2,3,4-tetrahydronaphth-2-yl)-5-aminomethyl-2,3-dihydro-2-thioxo-1H-imidazole hydrochloride (2a). The hydrochloride salt was prepared by the addition of ethereal HCl (1M, 20 mL, 20 mmol) to the free amine 2a (3.12 g, 10.6 mmol) which had been dissolved in MeOH (250 mL) by warming. The solvent was partially removed under reduced pressure and displaced by co-evaporation with EtOAc several times without evaporating to dryness. The resulting precipitate was treated with EtOAc (150 mL) and ether (150 mL), filtered off, washed with ether, and dried under nitrogen and then under high vacuum at 78° C. for 20 h to give the hydrochloride salt (3.87 g): mp 245° C. (dec); [a]²⁵_{D} +9.65° (c=1.70, DMSO); (93% ee by chiral HPLC); ¹H NMR (T=320° K, DMSO) δ 2.07 (m, 1H), 2.68-3.08 (m, 4H), 4.09 (m, 3H), 4.77 (m, 1H), 6.84 (m, 2H), 7.05 (s, 1H), 8.57 (br s, 3H), 12.4 (br s, 1H). Anal. Calcd for C₁₄H₁₆ClF₂N₃S.0.5H₂O: C, 49.33; H, 5.03; N, 12.33. Found: C, 49.44; H, 4.96; N, 12.18. The spectra for the (R)-enantiomer 2b are identical; mp 261-263° C.; [V]²⁵_{D} −10.8° (c=1.43, DMSO), 91.6% ee by chiral HPLC. Anal. Calcd for C₁₄H₁₆ClF₂N₃S.0.35H₂O: C, 49.73; H, 4.98; N, 12.42. Found: C, 49.80; H, 4.93; N, 12.39.

In vitro assay of DBH activity. DBH activity was assayed by measuring the conversion of tyramine to octopamine. Bovine DBH from adrenal glands was obtained from Sigma Chemical Co (St Louis, Mo.). Human secretory DBH was purified from the culture medium of the neuroblastoma cell line SK-N-SH. The assay was performed at pH 5.2 and 32° C. in 0.125 M NaOAc, 10 mM fumarate, 0.5-2 μM CUSO₄, 0.1 mg/mL catalase, 0.1 mM tyramine and 4 mM ascorbate. In a typical assay, 0.5-1 milliunits of enzyme were added to the reaction mixture and then a substrate mixture containing catalase, tyramine and ascorbate was added to initiate the reaction (final volume of 200 μL). Samples were incubated with or without the appropriate concentration of the inhibitor at 37° C. for 30-40 min. The reaction was quenched by the stop solution containing 25 mM EDTA and 240 μM 3-hydroxytyramine (internal standard). The samples were analysed for octopamine by reverse phase HPLC using UV detection at 280 nM. The remaining percent activity was calculated based on controls (without inhibitor), corrected using internal standards and fitted to a non-linear 4-parameter concentration-response curve to obtain IC₅₀ values.

In vitro assay of selected enzymes and neurotransmitter receptors. The activity of 2a at eleven different enzymes was determined using established assays (PanLabs Inc, Foster City, Calif.). The affinity of 2a for thirteen selected receptors was determined by radioligand binding assays using standard filtration techniques and membrane preparations. Binding data were analyzed by iterative curve fitting to a four parameter logistic equation. Kᵢ values were calculated using the Cheng-Prusoff equation.

In vivo biochemical studies in spontaneously hypertensive rats (SHR) and normal dogs. Mate SHR (15-16 week old, Charles River, Wilmington, Mass.) were used in the study. On the day of the study, the animals were weighed and randomly assigned to receive either placebo (vehicle) or the appropriate dose of 2a, 2b or 1. Each rat was dosed orally three times, 12 h apart, beginning in the morning. At 6 h after the third dose, the rats were anesthetized with halothane, decapitated, and the tissues (cerebral cortex, mesenteric artery and left ventricle) were rapidly harvested, weighed, placed in iced 0.4 M perchloric acid, frozen in liquid nitrogen and stored at −70° C. until analysis. Tissue NE and DA concentrations were assayed by HPLC using electrochemical detection.

Male beagle dogs (10-16 kg, Marshall Farms USA Inc, North Rose, N.Y.) were used in the study. On the day of the study, dogs were randomly assigned to receive either placebo (empty capsule) or the appropriate dose of 2a. Each dog was dosed twice a day for 4.5 days. 6 h after the first dose on day 5, the dogs were euthanized with pentobarbital and the tissues (cerebral cortex. renal artery, left ventricle) harvested, weighed, placed in iced 0.4 M perchloric acid, frozen in liquid nitrogen and stored at −70° C. until analysis. Tissue NE and DA concentrations were assayed by HPLC using electrochemical detection.

A separate study was conducted in dogs to determine the effects of chronic administration of 2a on the plasma DA/NE ratio. Animals were randomized to receive, orally, either placebo (empty capsule) or 2a (2 mg/kg, b.i.d) for 14.5 days. Daily blood samples were drawn, 6 h after the first dose, for the measurement of plasma concentrations of DA and NE. The samples were collected in tubes containing heparin and glutathione, centrifuged at −4° C. and stored at −70° C. until analysis.

Hemodynamic study in SHR. Male SHR (15-16 week old) were used in the study. The animals were lightly anesthetized with ether and the left femoral artery and vein were catheterized for measurement of blood pressure and drug administration, respectively. The animals were placed in restrainers and allowed to recover for 30-40 min. After obtaining baseline measurements, the animals were treated, orally, with either vehicle or the appropriate dose of 2a and hemodynamic parameters were continously recorded for 4 h. The animals were then anesthetized with pentobarbital, placed on a heating pad (37° C.) and ventilated with a Harvard rodent ventilator. After administration of atropine (1 mg/kg, iv) and tubocurarine (1 mg/kg, iv), the animals were pithed through the orbit of the eye with a stainless steel rod. The pithing rod was stimulated electrically with 1 ms pulses of 80V at different frequencies (0.15, 0.45, 1.5, 5, 15 Hz) to obtain frequency-pressor response curves.

### Example 7

Concentrations of dopamine and norepinephrine were determined in 942 samples of plasma collected from congestive heart failure (CHF) patients. The objectives of the study were:

1. to evaluate the effects of various doses of nepicastat on transmyocardial (arterial-coronary sinus) and coronary sinus catecholamine levels after four weeks, and to evaluate the safety and tolerability of nepicastat over 12 weeks.

2. to evaluate the effects of nepicastat on changes from baseline in:
a) Plasma (venous) catecholamine levels after four weeks and 12 weeks
b) Quality of life (QoL), CHF symptoms, Global Assessments, and NYHA class after four weeks and 12 weeks
c) Hemodynamic parameters, including cardiac output, systemic vascular resistance, MVO₂, pulmonary artery pressures, and pulmonary artery wedge pressure after four weeks
d) Hospitalizations and changes in medication dosages for the treatment of CHF over 12 weeks
e) Blood pressure and heart rate at four and 12 weeks
f) Six-Minute Walk Test after four weeks and 12 weeks
g) Left ventricular ejection fraction, left ventricular end systolic, and left ventricular end diastolic volumes at 12 weeks.

Samples of blood were collected from patients from a peripheral vein, whilst they were supine, at 2 hours post-dose during weeks 4 and 12. Further samples from supine patients were collected on day 0 (i.e. the day prior to the start of dosing) at a time corresponding to 2 hours post-dose. In addition, a group of patients underwent right heart and coronary sinus catheterization during week 4 at 2 hours post-dose and on day 0 (i.e. the day prior to the start of dosing) at a time corresponding to 2 hours post-dose. Triplicate samples of blood were collected from the arterial vein and coronary sinus of these patients.

Concentrations of the free base of dopamine and norepinephrine were determined by a radioenzymatic method. The method involves the incubation of the plasma samples with catechol-O-methyl transferase and tritiated S-adenosyl methionine. On completion of the incubation, the O-methylated catecholamines are extracted from the plasma by liquid/liquid extraction and then separated by thin layer chromatography. The relevant bands for each catecholamine are marked and then scraped into scintillation vials for counting. The quantitation limit of the method is 1 pg of dopamine or norepinephrine per mL of plasma. The linear range is 1 to 333000 pg of dopamine or norepinephrine per mL of plasma using aliquots of 0.045 mL to 1 mL. A complete description of the method can be found in the publication “Radioenzymatic Microassay for Simultaneous Estimations of Dopamine, Norepinephrine and Epinephrine in Plasma, Urine and Tissues” by Benedict et al (Clinical Chemistry, Vol. 31, No. 11, 1985, pp. 1861-1864).

A pooled human plasma sample was used as the Quality Control sample (QC) and was analyzed in singlicate each day during routine use of the method to monitor the performance of the method.

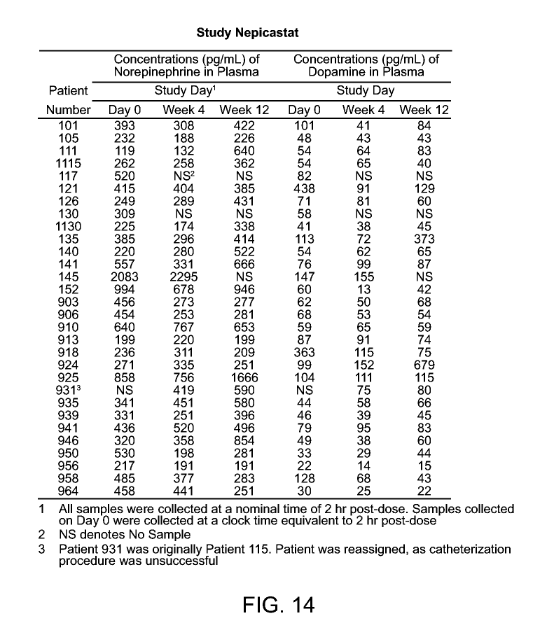

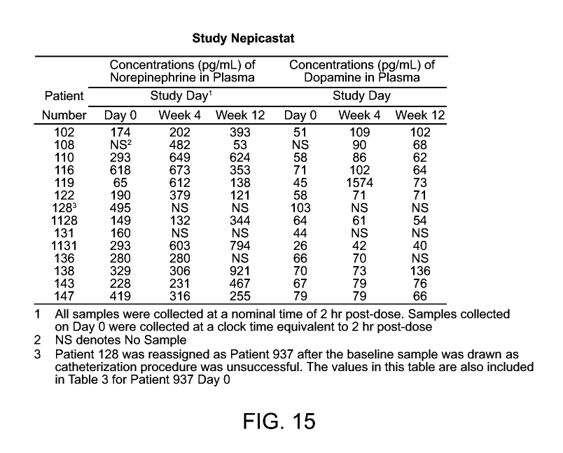

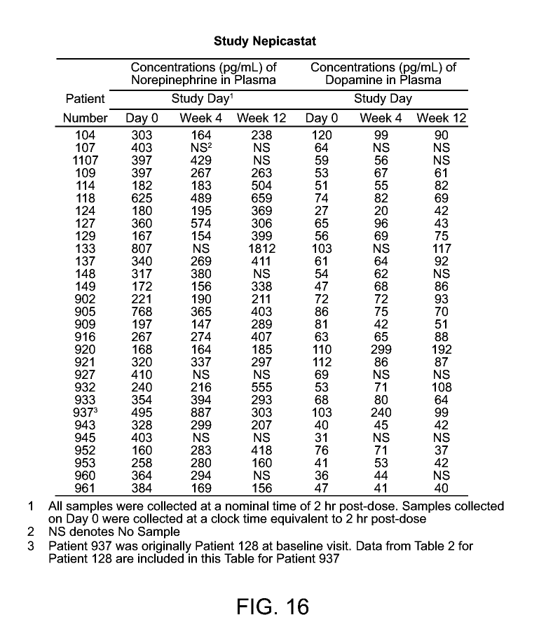

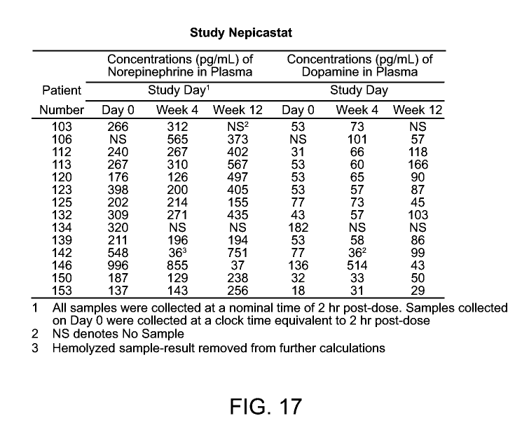

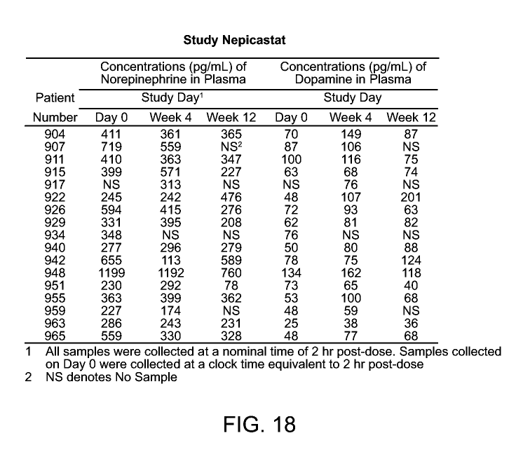

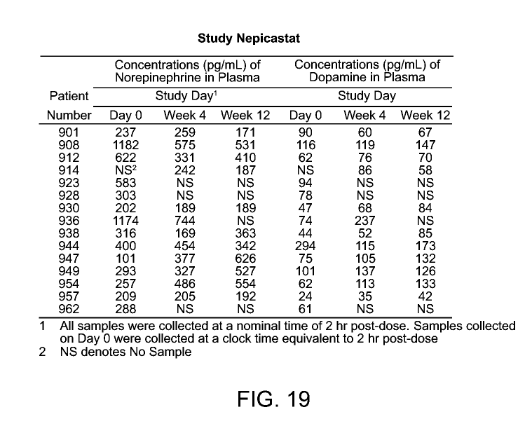

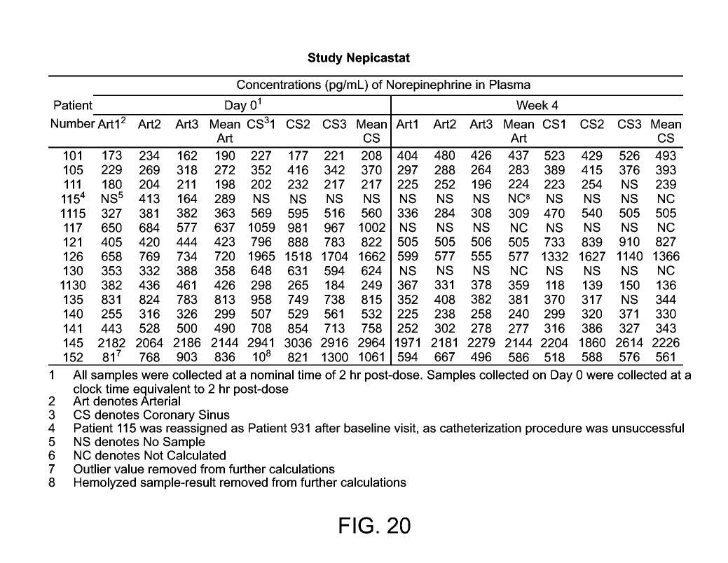

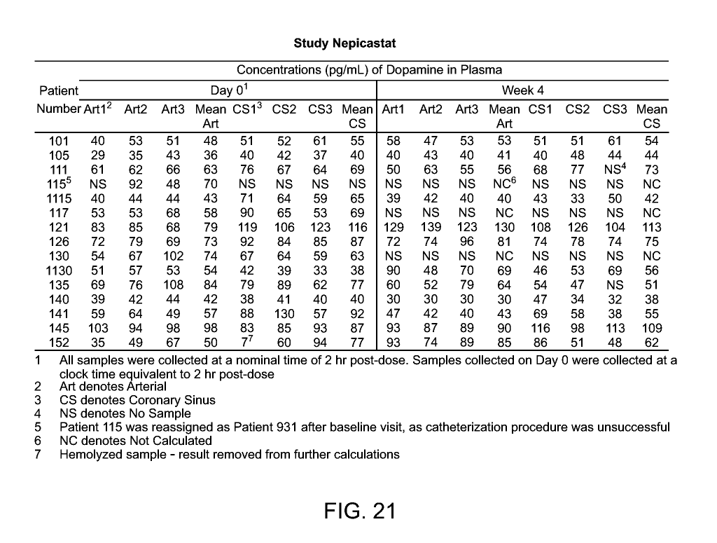

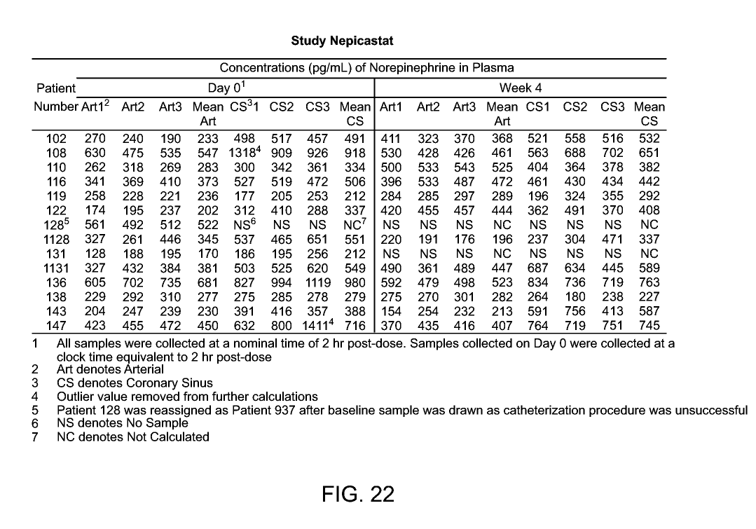

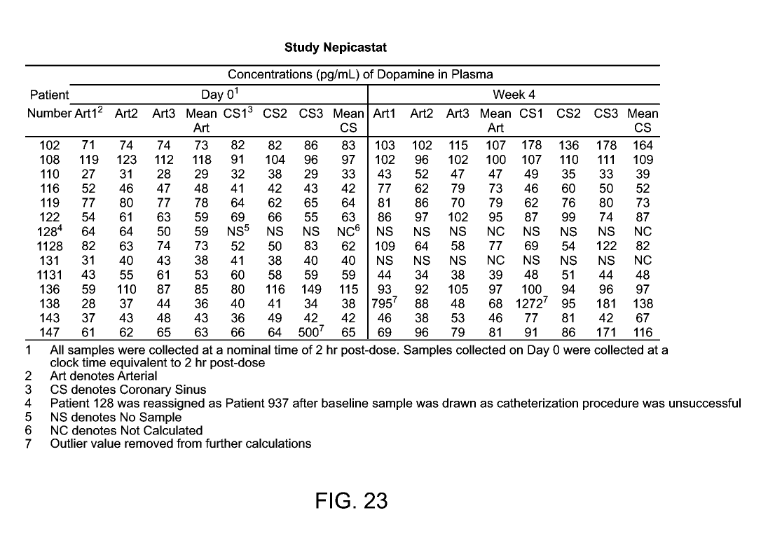

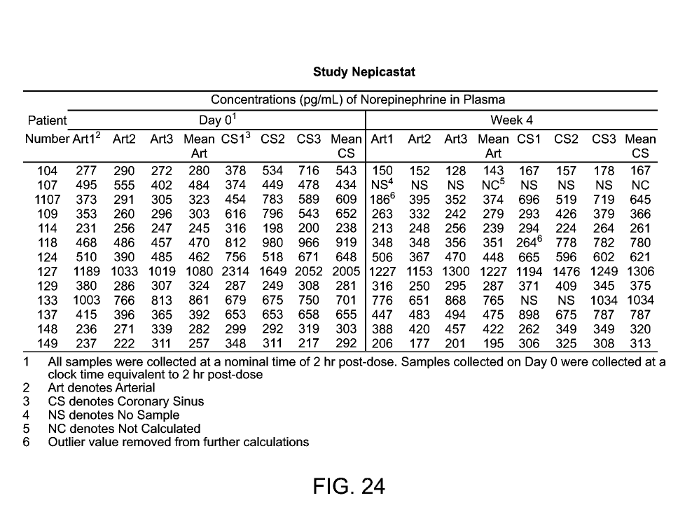

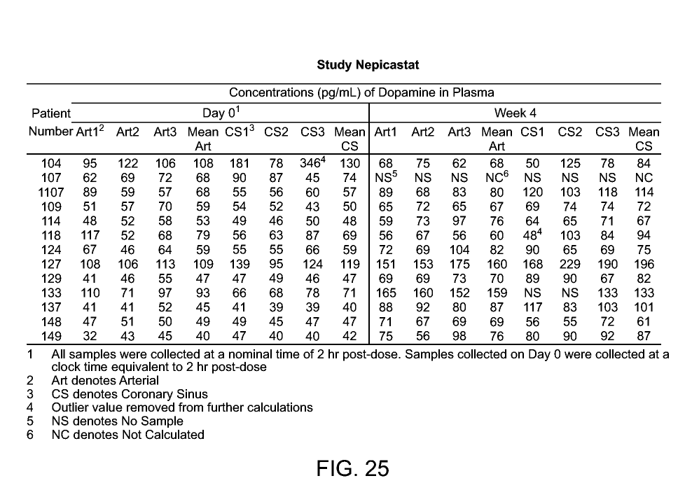

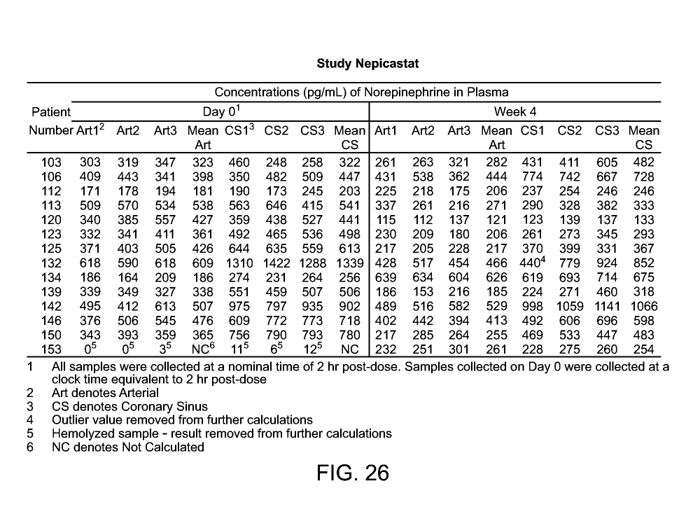

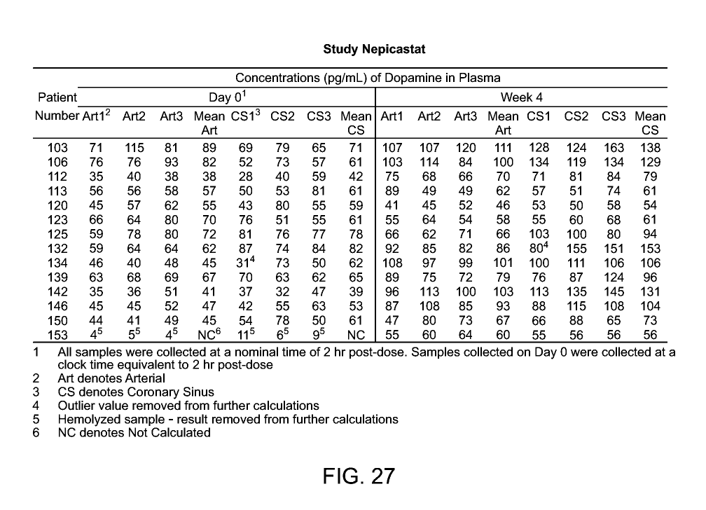

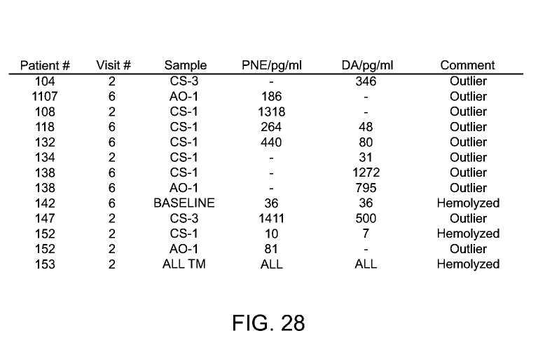

The analytical results have been unrandomized and are presented in FIGS. 14-27. Upon completion of the analysis of the samples, the results were reviewed. FIG. 28 shows a table that denotes data that should be discounted from further statistical analysis together with the reason for such an action.

### Example 8

Preclinical in vitro and in vivo pharmacology studies were conducted with nepicastat. The in vitro studies assessed the ability of the compound to inhibit DBH activity, and its binding affinity at selected receptors. The in vivo studies are subdivided into four categories: 1) biochemical effects (i.e. the ability to decrease tissue norepinephrine levels and increase dopamine levels), 2) effects on thyroid function, 3) cardiovascular effects, and 4) behavioral effects.

Nepicastat was a potent inhibitor of both bovine and human DBH. The IC₅₀ for nepicastaton human DBH was 9 nM (CL 6960), significantly lower than that for the DBH inhibitor SKF102698 (85 nM). The S enantiomer of RS-nepicastat (denoted as RS-nepicastat-197) was more potent than the R enantiomer (18 nM), denoted as (R)-5-Aminomethyl-1-(5,7-difluoro-1,2,3,4-tetrahydronaphth-2-yl)-2,3-dihydro-2-thioxo-1H-imidazole hydrochloride.

The binding affinity for nepicastat was screened at selected receptors. Nepicastat showed a binding affinity of less than 5.0 for M1, D1 and D2, and 5HT_{1A, 2A} and 2_{C}. The N-acetyl metabolite of nepicastat in rats and monkeys, showed a similar lack of binding affinity for these receptors. Thus nepicastat and its primary metabolite RS-47831-007 were not potent inhibitors for the receptors listed above.

The aortic contractile response in vitro to phenylephrine is impaired in spontaneously hypertensive rats (SHR) relative to normotensive Wistar-Kyoto rats. Daily treatment with nepicastat (10 mg/kg, p.o.) in SHR for 21 days restored phenylephrine responsiveness to values comparable to the Wistar-Kyoto rats.

Overall, nepicastat was an effective inhibitor of DBH in rats and dogs. Oral or intravenous administration resulted in a significant (p<0.05) decrease in tissue norepinephrine, an increase in dopamine, and an increase in the dopamine/norepinephrine levels in the heart, mesenteric or renal artery, and the cerebral cortex in both species.

In studies with male spontaneously hypertensive rats (SHR), nepicastat significantly decreased norepinephrine and increased dopamine and the dopamine/norepinephrine ratio in the mesenteric artery from 0.5 to 4 hours following oral or i.v. administration at 6.2 mg/kg. Significant changes in these parameters were also observed in the left ventricle of male Sprague-Dawley rats 6 hours after the second of two i.v. injections (15 mg/kg) given 12 hours apart. The 24 hour time course of tissue catecholamines was studied in male SHR following oral administration of either 10 or 30 mg/kg, respectively. The increase in the dopamine/norepinephrine ratio was significant at 1 hour, and was long lasting (12 hours at 10 mg/kg, mesenteric artery, and 24 hours at 30 mg/kg, left ventricle). Significant changes in mesenteric artery dopamine and norepinephrine levels were observed following 10 days of dosing to male Sprague-Dawley rats at 2.0 and 6.2 mg/kg p.o. b.i.d., with no significant effects observed in the cerebral cortex. SHR dosed at 1 or 10 mg/kg/d p.o. for either 7 or 25 days had significant increases in dopamine and the dopamine/norepinephrine ratio in the mesenteric artery and cerebral cortex. Taken together, nepicastatresulted in a significant decrease in norepinephrine and an elevation in dopamine and the dopamine/norepinephrine ratio in the mesenteric artery in rats with either acute or chronic (up to 25 days) dosing.

The effects of nepicastat in male SHR and Sprague-Dawley rats were found to be dose responsive when assessed 6 hours following a single oral dose at 0.3, 1, 3, 10, 30, and 100 mg/kg. In SHR there were significant changes in the dopamine/norepinephrine ratio in the mesenteric artery at doses of 0.3 mg/kg, in the left ventricle at 3.0 mg/kg, and in the cerebral cortex at 10 mg/kg. In Sprague-Dawley rats there were significant increases in the dopamine/norepinephrine ratio in the mesenteric artery at 3.0 mg/kg, in the left ventricle at 1.0 mg/kg, and in the cerebral cortex only at 100 mg/kg. In a second dose-response study in SHR, three doses were administered 12 hours apart at either 3.0, 10, 30, or 100 mg/kg, and tissue was harvested six hours after the third dose. nepicastat caused a significant dose dependent decrease in norepinephrine (10 mg/kg) and increase in dopamine (3.0 mg/kg) and the dopamine/norepinephrine ratio (3.0 mg/kg) in the left ventricle and mesenteric artery. The effects of nepicastat on dopamine and norepinephrine concentrations, and the dopamine/norepinephrine ratio in the cerebral cortex were significant only at 30 and 100 mg/kg. Similar significant dose-response effects in the left ventricle were seen in female Wistar rats dosed with nepicastat for 7 days via the drinking water (0.3, 0.6, and 1.0 mg/ml). In conclusion, nepicastat was less potent in inhibiting DBH in the cerebral cortex of rats (60-100 mg/kg/d) than in the left ventricle and mesenteric artery (1-6 mg/kg/d).

Nepicastat (the S enantiomer) was significantly more potent then the R enantiomer in the left ventricle and mesenteric artery in SHR after three doses given 12 hours apart (30 mg/kg p.o.). nepicastat was significantly more potent than the DBH inhibitor SKF102698 in decreasing norepinephrine and increasing dopamine and the dopamine/norepinephrine ratio in the left ventricle and mesenteric artery in SHR after a single dose, or three doses at 30 mg/kg. The potency relationships in the left ventricle and mesenteric artery resulting from these in vivo studies strongly parallel those obtained from in vitro studies using purified DBH (see above). However, nepicastat had significantly less effects than SKF 102698 in decreasing norepinephrine levels and increasing dopamine levels in the cerebral cortex. Norepinephrine has been shown to stimulate the release of renin and increase plasma renin activity. It was therefore of interest to assess whether decreasing norepinephrine levels with nepicastatwould result in a decrease in plasma renin activity. However, nepicastat (30 and 100 mg/kg/d p.o. for 5 days) did not alter plasma renin activity in male SHR. Thus, nepicastat, when given at doses that lower tissue norepinephrine levels, does not alter plasma renin activity in SHR.

Nepicastatcaused a significant decrease in norepinephrine levels and an increase in the dopamine/norepinephrine ratio, but did not alter dopamine levels, in the mesenteric artery from male beagle dogs 5 hours after administration of 30 mg/kg intraduodenally. When nepicastat was given to male beagle dogs for 4.5 days (5, 15, and 30 mg/kg b.i.d., or 10, 30, and 60 mg/kg/d) there was a significant decrease in norepinephrine, and an increase in dopamine and the dopamine/norepinephrine ratio in the renal artery, renal cortex, and renal medulla, with a plateau in response beginning at 10 mg/kg/d and extending through 60 mg/kg/d. Similar results were observed in the left ventricle, except that there was no significant increase in dopamine. In the cerebral cortex, norepinephrine significantly decreased at 30 and 60 mg/kg/d, and dopamine and the dopamine/norepinephrine ratio significantly increased at all doses. In conclusion, nepicastatwas a potent, orally active inhibitor of DBH in dogs at doses of at least 10 mg/kg/d.

Nepicastat has structural similarities to methimazole, a potent inhibitor of thyroid peroxidase in vivo. nepicastatat doses of 4 or 12.4 mg/kg/d, p.o. had no effect on serum levels of triiodothyramine or thyroxine in male Sprague-Dawley rats fed a low iodine diet and dosed for 10 days, while methimazole (2 mg/kg/d) significantly reduced serum levels of triiodothyramine or thyroxine. Thus, epicastat, unlike methimazole, did not affect serum levels of triiodothyramine or thyroxine.

Nepicastat induced a significant antihypertensive effect for up to 4 hours in conscious, restrained SHR (1.0-30 mg/kg, p.o.), and significantly reduced heart rate (10 and 30 mg/kg). The antihypertensive effects of nepicastatin conscious, restrained SHR (10 mg/kg, p.o.) were not attenuated by pretreatment with the dopamine receptor (DA-1) antagonist SCH-23390. nepicastat (10 mg/kg) also reduced blood pressure 4 hours after dosing in conscious, restrained normotensive Wistar-Kyoto rats; however, the decrease in pressure was less (−13 mmHg) than with SHR (−46 mmHg). To summarize together, nepicastat causes a decrease in blood pressure in both SHR and normotensive rats, though the antihypertensive effect is more pronounced in SHR. The antihypertensive effects in SHR do not appear to be mediated via DA-1 receptors.

Nepicastat also significantly attenuated the hypertensive and tachycardic responses to preganglionic nerve stimulation in pithed SHR 5 hours after dosing (3 mg/kg p.o.). Thus, nepicastat reduces the rise in blood pressure in response to sympathetic nerve stimulation. Acute intravenous treatment of anesthetized SHR with nepicastat (3.0 mg/kg, i.v.) decreased mean arterial pressure over a 3 hour period, but did not lower renal blood flow or alter urine production or urinary excretion of sodium or potassium. The calculated renal vascular resistance was decreased following dosing. An attempt was made using the DA-1 antagonist SCH-23390 to assess if the renal vasodilatory effects of nepicastat were mediated by DA-1 receptors. However this compound reduced blood pressure when given alone, thus making the results uninterpretable. Overall, nepicastat did not impair renal function in anesthetized SHR, and did not decrease renal blood flow despite causing a decrease in arterial blood pressure.

Daily treatment with nepicastat (1 and 10 mg/kg, p.o.) in SHR for 21 days did not alter heart rate, or systolic blood pressure as measured by the tail cuff method. However, nepicastat (10 mg/kg, p.o.) induced a significant antihypertensive effect when the rats were restrained and their blood pressure measured directly via an arterial cannulae.

nepicastat significantly lowered blood pressure in SHR instrumented with radio-telemetry blood pressure transducers at doses of 30 and 100 mg/kg/d for 30 days, but produced no significant effects were observed at 3 and 10 mg/kg/d. The effect at 30 and 100 mg/kg/d persisted over a 24-hour period after a single dose, and there was no loss of effect over 30 days. Heart rate was not increased, and motor activity was unaffected. A combination of a dose of the angiotensin converting enzyme inhibitor enalapril (1 mg/kg, p.o.) that failed to lower blood pressure with nepicastat (30 mg/kg) caused a potentiation of the antihypertensive effects of nepicastat over 30 days of dosing, and resulted in a significant reduction in left ventricular mass. A reduction in left ventricular mass did not occur with enalapril alone. Thus, 30 days of treatment of SHR with nepicastatat 30 and 100 mg/kg/d resulted in a decrease in blood pressure and, when combined with enalapril, additional blood pressure decreases along with a reduction in left ventricular mass.

The blood pressure lowering effect of nepicastatin normotensive Wistar rats instrumented with radio-telemetry blood pressure transducers was less than the effect observer in SHR at doses of 30 and 100 mg/kg/d for 7 days. At 30 mg/kg/d the peak decrease in blood pressure was −10 mmHg, compared to −20 in SHR. At 100 mg/kg/d the peak decrease in blood pressure was −17 mmHg, compared to −42 in SHR. Thus, nepicastat had a greater blood pressure lowering effect in SHR than in normotensive rats.

Studies in normal anesthetized dogs showed no cardiovascular effects of nepicastat following acute intravenous dosing (1-10 mg/kg i.v.) with no changes in arterial blood pressure, left ventricular pressures (including peak dp/dt), heart rate, cardiac output or renal blood flow for up to five hours after dosing. A similar lack of effect was observed in chronically instrumented, conscious dogs studied for 12 hours after a single dose (3-30 mg/kg i.v.).

Nepicastat (30 mg/kg intraduodenally) did not significantly inhibit either the decrease in renal blood flow in response to direct renal nerve stimulation, or the increase in arterial blood pressure in response to carotid artery occlusion up to 5 hours after dosing in anesthetized male beagle dogs. However, nepicastat caused a significant decrease in norepinephrine levels and an increase in the dopamine/norepinephrine ratio, but not dopamine levels, in the mesenteric artery 5 hours after dosing. Thus, although tissue norepinephrine levels were significantly reduced, there was no significant inhibition of sympathetically-evoked functional responses.

When nepicastat was given to male beagle dogs for 4.5 days at 10 mg/kg/d there was no statistically significant decrease in the degree of blood pressure and heart rate increases in response to carotid artery occlusion in anesthetized animals. nepicastat treatment significantly reduced the increase in heart rate in response to an i.v. tyramine challenge, but produced only slight and non-significant inhibition of blood pressure increases. Thus, chronic dosing with nepicastatat at a dose that has been shown to result in a maximal decrease in tissue norepinephrine levels, does not have a major inhibitory effect on sympathetically-evoked functional responses.

Nepicastat caused no significant effects on gross motor behavior in mice following acute dosing at 1.0-30 mg/kg, p.o., and it did not effect locomotor activity in mice (10-100 mg/kg i.p.). Acute administration to rats did not effect locomotor activity or acoustic startle reactivity (3-100 mg/kg i.p.).

No behavioral effects were observed in rats following 10 days of dosing at 10, 30, and 100 mg/kg/d, p.o. (AT 6867). Rectal temperature was also unaffected. Motor activity and auditory startle reflex were significantly reduced by treatment with the DBH inhibitor SKF102698 (100 mg/kg/d, p.o.), and by the centrally acting a-adrenergic agonist clonidine (20 mg/kg, b.i.d., p.o.). Motor activity was also unaffected over 30 days of dosing in SHR (3-100 mg/kg/d, p.o.) (AT 6829). Thus, nepicastat did not cause detectable changes in central nervous system mediated behavioral effects in rats.

Nepicastat is a potent competitive inhibitor of human DBH in vitro, and in rats and dogs in vivo. In rats, oral treatment with nepicastat resulted in significant evidence for DBH inhibition in the heart and mesenteric artery at a dose 6 mg/kg/d. In contrast to another DBH inhibitor, SKF 102698, nepicastat showed some selectivity to the left ventricle and mesenteric artery relative to the cerebral cortex. No behavioral effects were observed with nepicastat in rats. In dogs, a plateau effect for DBH inhibition occurred at 10 mg/kg/d in the heart, renal artery and kidney; the minimal dose for significant effects has not been identified. nepicastat significantly reduced the hypertensive response to sympathetic nerve stimulation in rats (3 mg/kg p.o.), and it significantly lowered blood pressure throughout the day when dosed once daily (30 mg/kg/d p.o.) for 30 days in SHR. In conclusion, nepicastat is a potent DBH inhibitor that modulates the action of the sympathetic nervous system.

### Example 9

The studies described here were designed to evaluate the pharmacokinetics of higher oral doses of nepicastat, to compare the pharmacokinetics in male and female rats, and to determine penetration of nepicastat into the CNS by quantitating levels of nepicastat in brain.

Male rats (Crl: CD BR Vaf+) weighing 180-220 g were fasted overnight before dosing and until 4 hr after dosing. Doses were formulated in water containing 2% 1-hydroxypropyl methylcellulose (50 centipoises viscosity), 1% benzyl alcohol, and 0.6% Tween 80 (all obtained from Sigma Chemical Company). Concentration of drug in the dose solutions was 5, 15, and 50 mg/ml for the 10, 30, and 100 mg/kg doses, respectively, and was verified by liquid chromatography (LC). The 5 mg/ml dose was a clear solution and the higher concentrations were a translucent suspension. Dose volumes were 2.0 ml/kg. At various times after dosing, samples of blood were obtained by cardiac puncture with heparinized syringes, and plasma was prepared by centrifugation. Brains of rats were surgically excised, and all samples were frozen at −20° C. until analysis.

Aliquots of plasma (0.05 or 0.5 ml) were mixed with internal standard (50 μl of methanol containing 5 μg/ml a monofluoro analog of nepicastat, and 5 mg/ml dithiothreitol). Samples were mixed with 200 mM sodium phosphate buffer, pH 7.0, (0.5 ml) and extracted with 3 ml of ethyl acetate/hexane (1/1, v/v). The organic phase containing analytes was back extracted with 250 μl of 250 mM acetic acid and 100 μl aliquots of the aqueous phase were assayed by LC. The LC system used a Keystone Hypersil BDS 15 cm C₈ column at ambient temperature. Mobile phase A was 12.5 mM potassium phosphate, pH 3.0, with 5 mM dodecanesulfonic acid and mobile phase B was acetonitrile. Solvent composition was 40% B and was pumped at a flow rate of 1 ml/min. Detection was by UV absorption at 261 nm. Concentrations of analytes were determined from a standard curve generated from the analysis of plasma from untreated rats fortified with known concentrations of analyte. Plasma concentration data are expressed as μg (free base) per ml.

Brains were rinsed briefly with saline, blotted on a paper towel, then weighed (1.5-2.0 g). Internal standard was added (50 μl of methanol containing 20 μg/ml a monofluoro analog of nepicastat), and brains were homogenized in 5 ml of 200 mM sodium phosphate, pH 7.0, containing 0.5 mg/ml dithiothreitol. Aliquots of homogenate (2 ml) were extracted with 10 ml of ethyl acetate/hexane (1/1, v/v). The organic phase was gently back extracted with 150 μl of 250 mM acetic acid.

Following addition of 100 μl of methanol to the aqueous phase (to disperse any emulsion), 100 μl aliquots were assayed by LC as described for plasma. Level in brain are expressed as μg (free base) per g of brain tissue.

Pharmacokinetic parameters were calculated from mean plasma concentrations. Plasma half-life (T_{1/2}) was calculated as 0.693/β, where β is the elimination rate constant determined by linear regression of the log plasma concentration vs. time data within the terminal linear portion of the data. Areas under the plasma concentration vs. time curve (AUC) from zero to the time of the last quantifiable plasma concentrations were calculated by the trapezoidal rule. AUC from zero to infinity (AUCₜₒₜₐₗ) was calculated as:

AUCₜₒₜₐₗ=AUC(0−Cₗₐₛₜ)+Cₗₐₛₜβ where Cₗₐₛₜ is the last quantifiable plasma concentration.

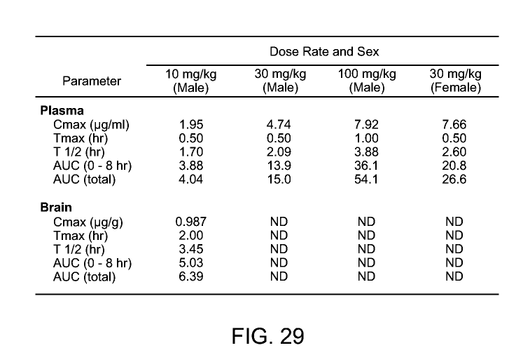

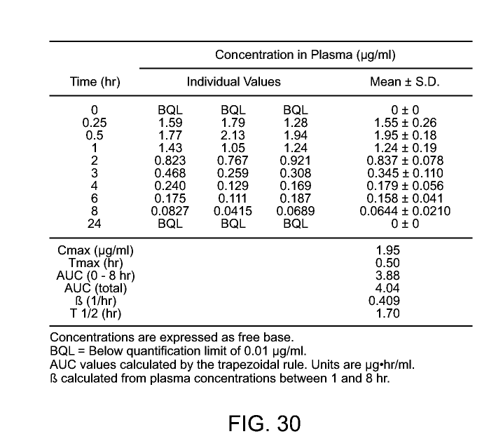

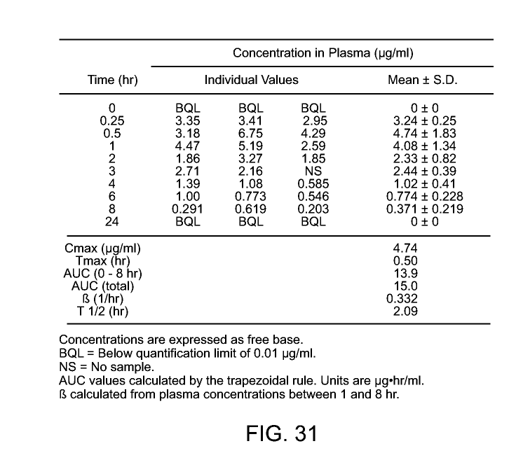

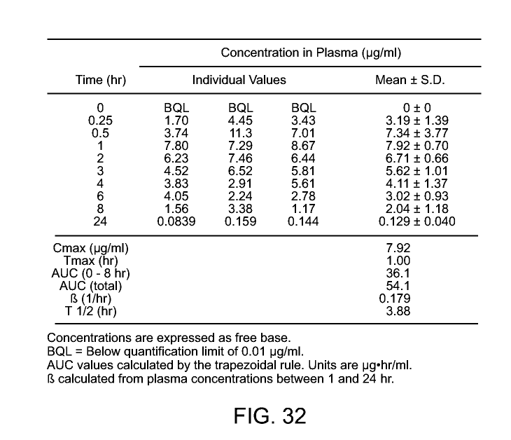

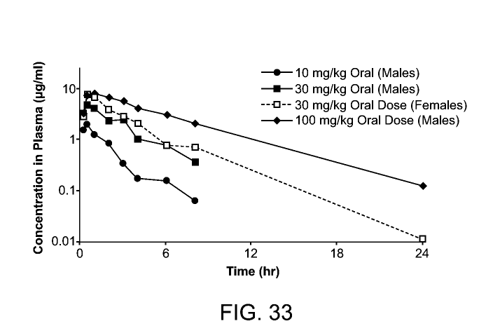

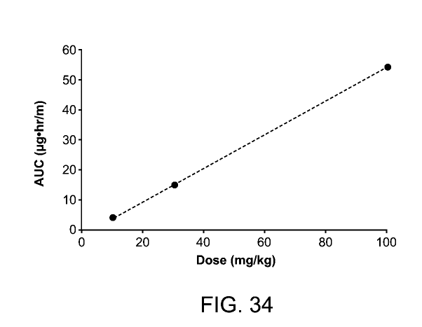

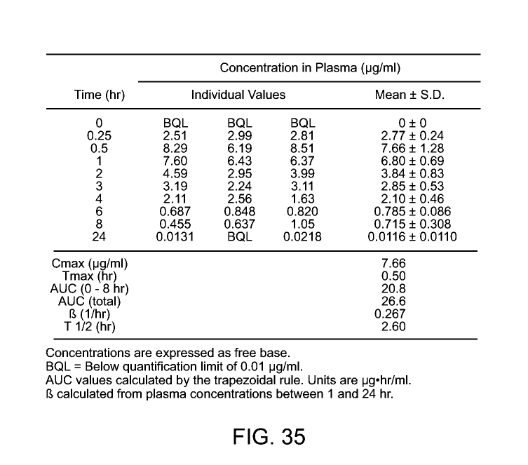

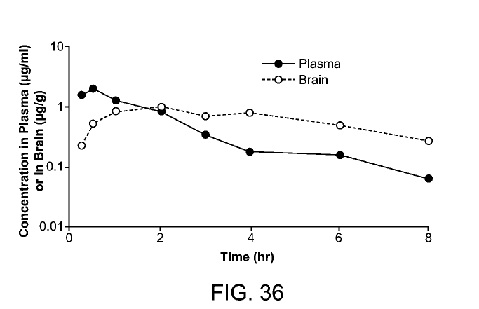

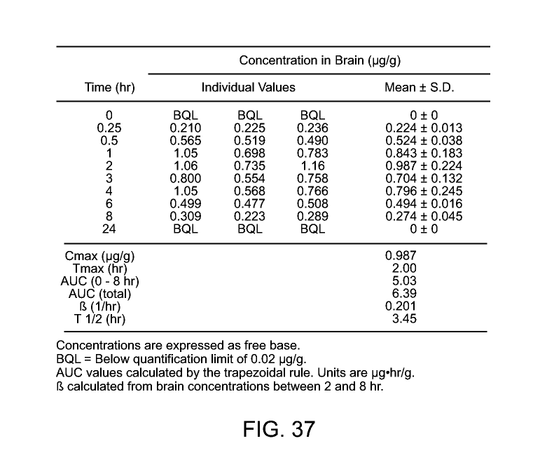

FIG. 29 shows pharmacokinetic parameters of nepicastat in rat plasma and brain. Concentrations of nepicastat in plasma of male rats given 10, 30, or 100 mg/kg single oral doses are shown in FIGS. 30-32 and plotted in FIG. 33. Concentrations of nepicastat in plasma increased with increasing dose, and the relationship between AUCₜₒₜₐₗ and dose was linear (FIG. 34). The elimination half-life appeared to increase slightly at higher doses (1.70, 2.09, and 3.88 hr following the 10, 30, and 100 mg/kg oral doses to male rats, respectively). Following a 30 mg/kg oral dose of nepicastat to female rats, the plasma AUCₜₒₜₐₗ of nepicastat was 77% higher in female rats than in male rats given an equivalent dose of nepicastat (FIGS. 33 and 35). Levels of nepicastat in brain (expressed as μg/g) were initially lower than those in plasma (expressed as μg/ml). From 2 hr following dosing onward, however, concentrations of nepicastat in brain exceeded those in plasma (FIGS. 36-37).

Plasma levels of nepicastat in male rats increased linearly with increasing doses between 10 and 100 mg/kg, based on values of AUCₜₒₜₐₗ.

Plasma levels of nepicastat were higher in female rats than in male rats following a 30 mg/kg oral dose.

Following administration of a 10 mg/kg oral dose of nepicastat to male rats, levels of nepicastat in brain were initially lower than those in plasma, but from 2 hr onward, levels of nepicastat in brain were greater than in plasma.

### Example 10

The purpose of this study was to determine the 24 hours time course of the effects of nepicastat (10 mg/kg) on dopamine and norepinephrine levels in the mesenteric artery following a single oral dose in spontaneously hypertensive rats. Catecholamine levels were measured at 1, 2, 4, 6, 8, 12, 16, and 24 hours after a single oral administration of either nepicastat (10 mg/kg) or vehicle (dH₂O; 10 ml/kg).

Sixteen-17 week old, male spontaneous hypertensive rats (SHR) rats weighing 300-400 grams were allowed food and water ad libitum. Animals were weighed and randomly assigned, the afternoon before the study, to one of the following treatment groups (n=9 per group): a single oral administration of nepicastat at 10 mg/kg or a single oral administration of vehicle (10 ml/kg) to be sacrificed at 1, 2, 4, 6, 8, 12, 16, or 24 hours. nepicastat was synthesized as the hydrochloride salt by the Institute of Organic Chemistry, Syntex Discovery Research and obtained from Syntex Central Compound Inventory. nepicastat was dissolved in vehicle (dH₂O) to yield an oral dose that could be administered in repeated volumes of 10 ml/kg. All doses of nepicastat were administered as free base equivalents and prepared the morning of administration. Animals were dosed every minute the morning of sacrifice. At 1, 2, 4, 6, 8, 12, 16 and 24 hours following administration, 9 treated animals and 9 vehicle animals were anesthetized with halothane, decapitated, and the left ventricle and mesenteric artery were rapidly harvested and weighed. The mesenteric artery was put in 0.5 ml of 0.4M perchloric acid in a centrifuge tube and the left ventricle put into an empty cryotube. Both tissues were immediately frozen in liquid nitrogen and stored at −70° C. Mesenteric artery catecholamine levels were determined using HPLC with electrochemical detection. At the time of decapitation, plasma samples were taken by draining blood from the carcass into a tube containing heparin, and centrifuging at 4° C.

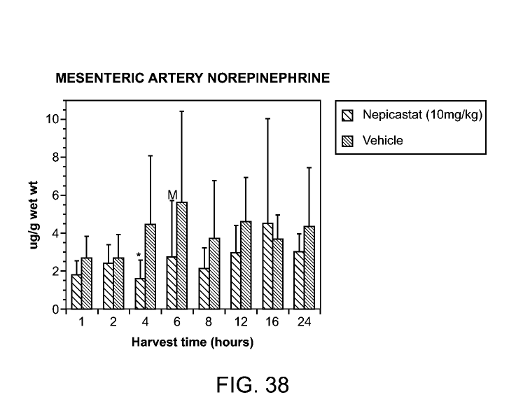

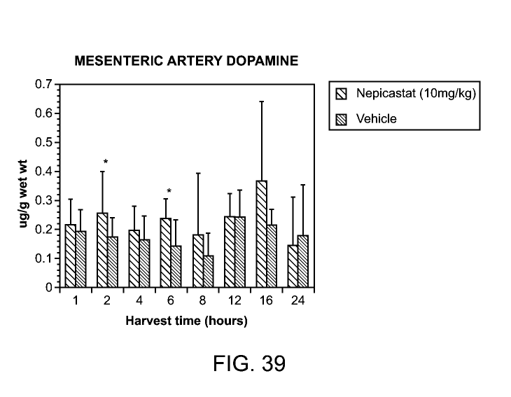

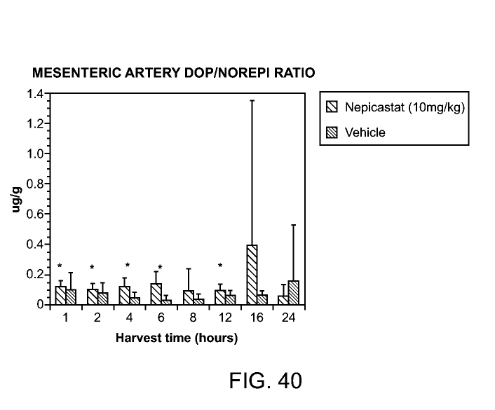

Each treatment group was compared to vehicle at each time point. A two way analysis of variance (ANOVA) with effects TRT, HARVEST and their interaction was performed. A one way ANOVA with factor TRT was performed for each harvest time. Pairwise analyses between treated and vehicle animals, at each time point, were carried out using Fisher's LSD strategy to control the experiment-wise error rate._Norepinephrine values were significantly (p<0.05) lower than vehicle only at the 4 hr time point. Levels were marginally (0.05<p<0.1) lower at the 6 hour time point (FIG. 38)._Dopamine levels were significantly (p<0.05) higher than those of vehicle at the 2 and 6 hr harvest times (FIG. 39). The dopamine/norepinephrine ratio was significantly (p<0.05) greater than those of vehicle treated animals at the 1, 2, 4, 6 and 12 hour time points (FIG. 40).

In general, nepicastat had few statistically significant effects on mesenteric artery norepinephrine or dopamine levels following a single oral administration at 10 mg/kg in spontaneously hypertensive rats at 1, 2, 4, 6, 8, 12, 16 or 24 hours following dosing. However, a consistent increase in the dopamine/norepinephrine ratios were observed across most of the first 12 hours of treatment. At the 16 and 24 harvest time no changes in any of the three parameters were observed.

### Example 11

The purpose of this study was to determine the effects of intravenous administration of nepicastat (hereafter referred to as nepicastat) on the levels of dopamine and norepinephrine in the left ventricle in Sprague-Dawley rats. Animals received two intravenous (iv) administrations, 12 hours apart, of either vehicle (75% propylene glycol+25% DMSO; 1.0 ml/kg) or 15 mg/kg of nepicastat. Tissue norepinephrine and dopamine levels were measured six hours after the last compound administration.

Sixteen to 17 week old male Sprague-Dawley rats, weighing 300-400 grams, were allowed food and water ad libitum. Animals were weighed and randomly assigned, the afternoon before the study, to one of the following treatment groups (n=10 per group): vehicle (1.0 ml/kg) or nepicastat at 15 mg/kg. Nepicastat was synthesized by the Institute of Organic Chemistry, Syntex Discovery Research and obtained from Syntex Central Compound Inventory. nepicastat was dissolved in the appropriate amount of vehicle (75% propylene glycol+25% DMSO) to obtain a dosing volume of 1.0 ml/kg. nepicastat was administered as the free base equivalent and prepared the afternoon prior to the first administration.

Each rat was dosed iv in the tail vein the afternoon before harvest. The dosing was repeated 12 hours later the following morning. Six hours after the final administration rats were anesthetized with halothane, decapitated, and the left ventricle was rapidly harvested and weighed. The ventricle was placed in 1.0 ml iced 0.4 M perchloric acid. Tissues were immediately frozen in liquid nitrogen and stored at −70° C. Tissue dopamine and norepinephrine concentrations were assayed by high performance liquid chromatography using electrochemical detection.

A one-way analysis of variance (ANOVA) with a main effect for treatment was performed for norepinephrine. A Kruskal-Wallis was performed for dopamine and their ratio primarily due to heterogeneous variances among treatment groups. Subsequent pairwise comparisons between nepicastat treated rats and vehicle were performed using Fisher's LSD test. A Bonferroni adjustment was performed on all p-values to ensure an overall experiment-wise type 1 error rate of 5%.

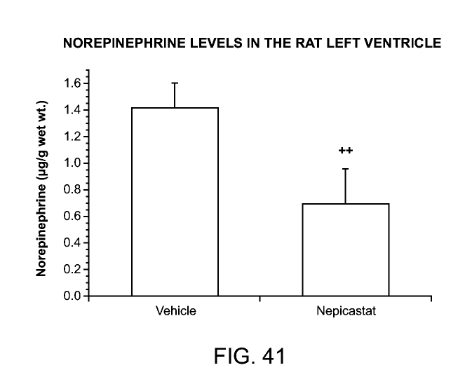

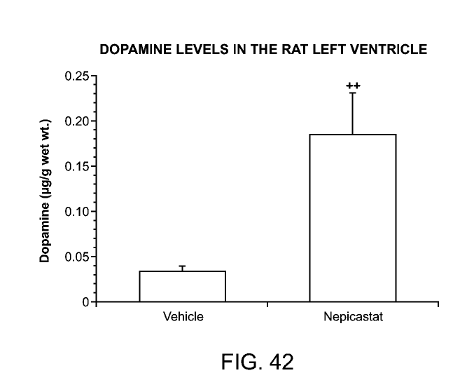

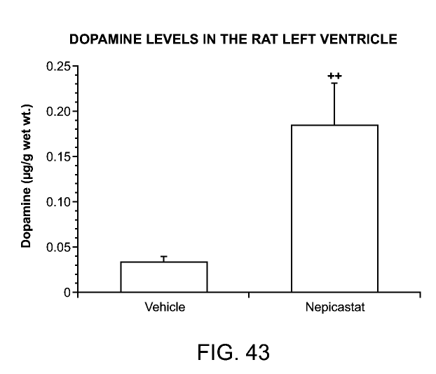

Nepicastat administered at 15 mg/kg significantly (p<0.01) decreased norepinephrine levels by 51% (FIG. 41), and significantly (p<0.01) increased dopamine levels by 472% (FIG. 42), and significantly (p<0.01) increased the dopamine/norepinephrine ratio by 1117% (FIG. 43), compared to vehicle treated animals.

In conclusion, intravenous administration of nepicastat resulted in significant inhibition of DBH in the left ventricle of Sprague-Dawley rats.

### Example 12

This study assessed the effectiveness of nepicastat in altering the levels of dopamine and norepinephrine in the cortex, left ventricle, and mesenteric artery of male spontaneously hypertensive rats (SHR). Animals were given three doses, 12 hours apart at 3, 10, 30 or 100 mg/kg p.o.

This study also compared the efficacy of the S enantiomer (nepicastat) with the R enantiomer ((R)-5-Aminomethyl-1-(5,7-difluoro-1,2,3,4-tetrahydronaphth-2-yl)-2,3-dihydro-2-thioxo-1H-imidazole hydrochloride) following three doses (30 mg/kg).

This study also compared the effects of nepicastat with SKF102698, a DBH inhibitor previously shown to be orally active in rats.

Compounds were prepared and administered as the free base equivalent. Nepicastat, (R)-5-Aminomethyl-1-(5,7-difluoro-1,2,3,4-tetrahydronaphth-2-yl)-2,3-dihydro-2-thioxo-1H-imidazole hydrochloride and SKF 102698 were obtained from Syntex Central Compound Inventory. Nepicastat was dissolved in the appropriate amount of vehicle (dH₂O for nepicastat and PEG 400:dH₂O, 50:50 vol:vol for SKF102698. Doses of 3, 10, 30, and 100 mg/kg of nepicastat, and 30 mg/kg SKF 102698 were prepared in 10.0 ml/kg dosing volumes.

Fifteen to sixteen week old male spontaneously hypertensive rats (SHR) (Charles River Labs) were allowed food and water ad libitum. Animals were weighed and randomly assigned to one of the following treatment groups: 1) distilled water vehicle (dH₂O), or nepicastat at 3, 10, 30, and 100 mg/kg, 2) (R)-5-Aminomethyl-1-(5,7-difluoro-1,2,3,4-tetrahydronaphth-2-yl)-2,3-dihydro-2-thioxo-1H-imidazole hydrochloride at 30 mg/kg in distilled water, or 3) PEG 400:dH₂O vehicle or RS-2643 1-000 at 30 mg/kg. Each rat was dosed orally (p.o., using a gavage needle) three times 12 hours apart, beginning in the morning. At six hours after the third dose rats were anesthetized with halothane, decapitated, and the cortex, mesenteric artery, and left ventricle were rapidly harvested, weighed, placed in iced 0.4 M perchioric acid, frozen in liquid nitrogen, and stored at −70° C. Tissue dopamine and norepinephrine concentrations were assayed by high performance liquid chromatography and electrochemical detection.

Four series of statistical analyses were performed. The first series compared the rats treated with various doses of nepicastat, and (R)-5-Aminomethyl-1-(5,7-difluoro-1,2,3,4-tetrahydronaphth-2-yl)-2,3-dihydro-2-thioxo-1H-imidazole hydrochloride at 30 mg/kg to the vehicle control animals. A nonparametric one-way analysis of variance (ANOVA) with factor Dose and blocking factor Day was performed for each tissue and strain separately. Overall results are reported. Pairwise analysis between treated and controls at each dose were carried out using Dunnett's test to control the experiment-wise error rate. The second statistical test compared SKF 102698 to the PEG-dH₂O vehicle treated group using a nonparametric t-test. The third statistical test compared (R)-5-Aminomethyl-1-(5,7-difluoro-1,2,3,4-tetrahydronaphth-2-yl)-2,3-dihydro-2-thioxo-1H-imidazole hydrochloride to NEPICASTAT at doses of 30 mg/kg using a nonparametric t-test. A fourth statistical analysis compared RS25560-197 to SKF 102698 at doses of 30 mg/kg. Since two different vehicles were used, a linear contrast was developed which calculates the difference of differences as follows:

Change=(30 mg/kg−Vehicle)_{NEPICASTAT}−(30 mg/kg−Vehicle)_{SKF102698}

This new variable was tested for equality to zero by the SAS procedure General Linear Models.

All data in the figures is presented ± the standard deviation.

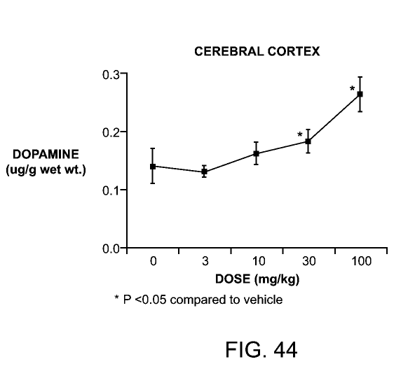

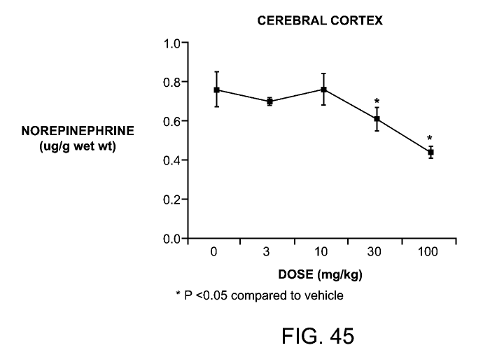

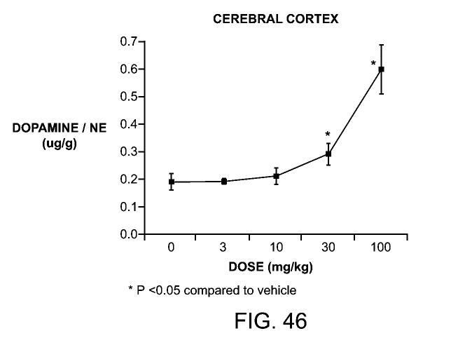

The dopamine concentration in the cerebral cortex was significantly (p<0.05) greater (FIG. 44), the norepinephrine concentration was significantly (p<0.05) lower (FIG. 45), and the dopamine/norepinephrine ratios significantly (p<0.05) greater (FIG. 46) than vehicle at doses of 30 and 100 mg/kg of nepicastat.

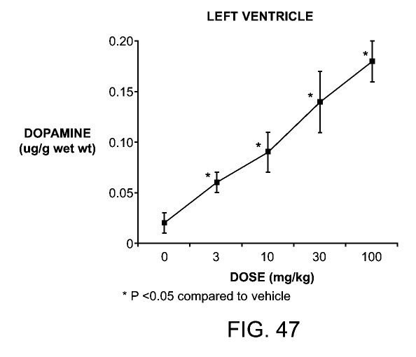

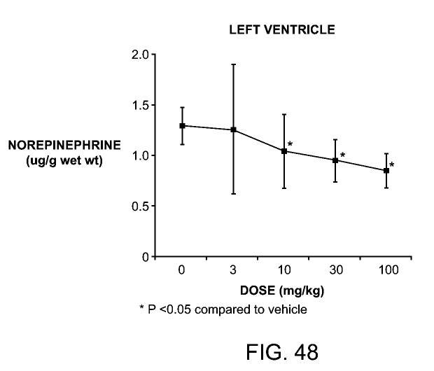

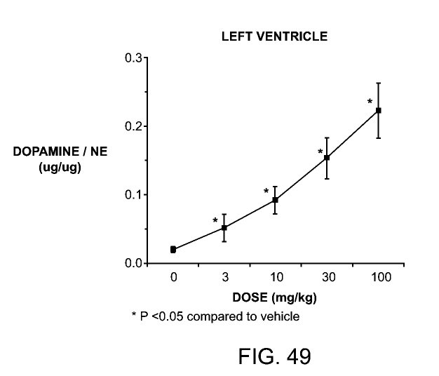

Dopamine concentration in the left ventricle was significantly (p<0.05) greater than vehicle at doses of 3, 10, 30 and 100 mg/kg (FIG. 47). Norepinephrine concentration was significantly (p<0.05) lower than vehicle at doses of 10, 30 and 100 mg/kg (FIG. 48). The dopamine/norepinephrine ratio in the left ventricle was significantly (p<0.05) greater than vehicle at doses of 3, 10, 30, and 100 mg/kg (FIG. 49) of nepicastat.

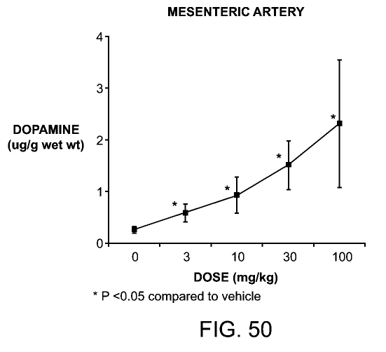

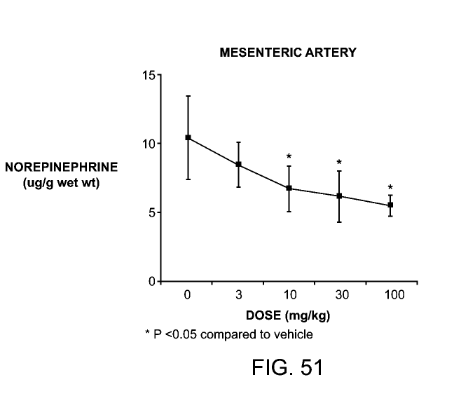

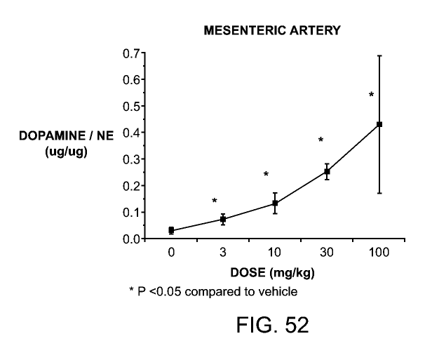

Dopamine concentration in the mesenteric artery of SHR was significantly (p<0.05) greater than vehicle at doses of 3, 10, 30 and 100 mg/kg (FIG. 50). Norepinephrine concentration was not significantly less (p>0.05) than vehicle at 10, 30, and 100 mg/kg (FIG. 51). The dopamine/norepinephrine ratios in the mesenteric artery were significantly (p<0.05) greater than vehicle at all doses (FIG. 52) of nepicastat.

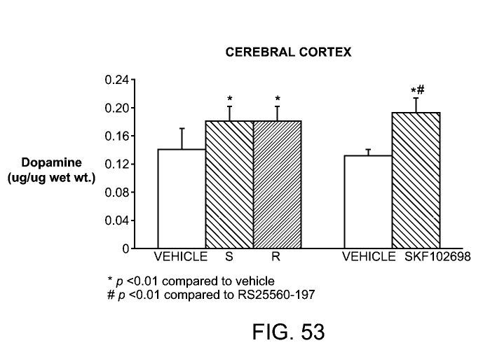

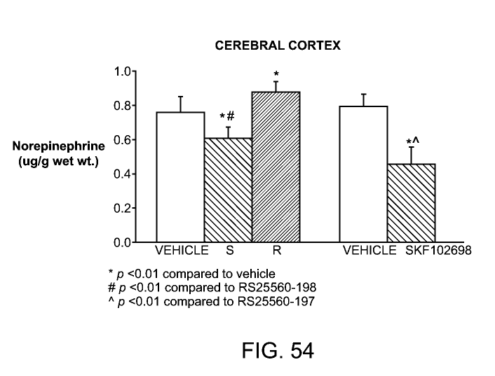

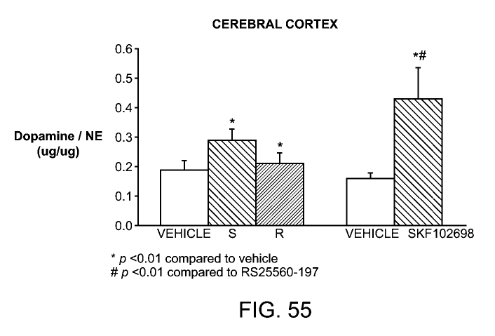

In the cerebral cortex, relative to treatment with vehicle, (R)-5-Aminomethyl-1-(5,7-difluoro-1,2,3,4-tetrahydronaphth-2-yl)-2,3-dihydro-2-thioxo-1H-imidazole hydrochloride resulted in significant increase in both dopamine(FIG. 53) and norepinephrine (FIG. 54) (p<0.01), and had no effect on the dopamine/norepinephrine ratio (FIG. 55). Norepinephrine levels were significantly lower with nepicastat compared to (R)-5-Aminomethyl-1-(5,7-difluoro-1,2,3,4-tetrahydronaphth-2-yl)-2,3-dihydro-2-thioxo-1H-imidazole hydrochloride (p<0.01)(FIG. 54).

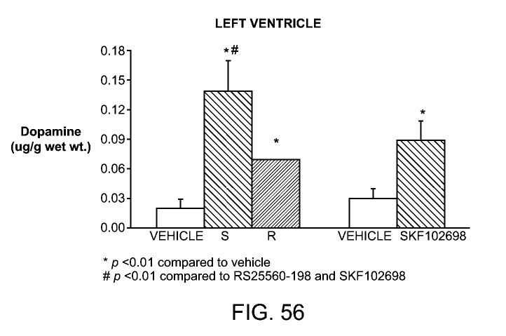

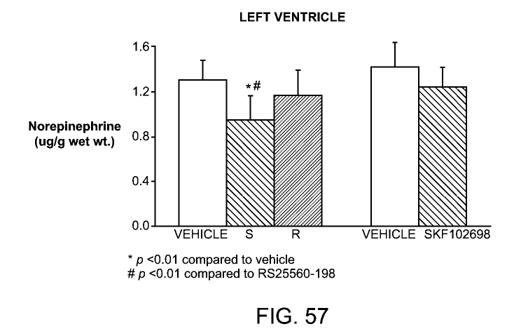

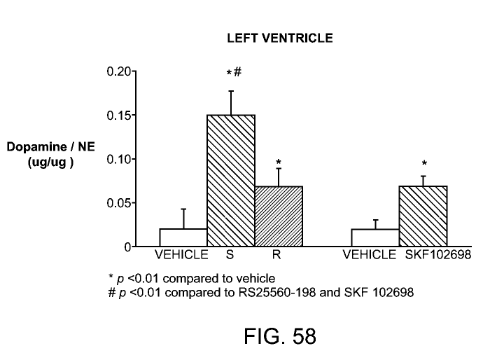

In the left ventricle, relative to treatment with vehicle, (R)-5-Aminomethyl-1-(5,7-difluoro-1,2,3,4-tetrahydronaphth-2-yl)-2,3-dihydro-2-thioxo-1H-imidazole hydrochloride resulted in a significant increase in dopamine (FIG. 56) and the dopamine/norepinephrine ratio (FIG. 58) (p<0.01), but did not significantly lower norepinephrine levels (FIG. 57). NEPICASTAT was significantly more effective (p<0.01) than (R)-5-Aminomethyl-1-(5,7-difluoro-1,2,3,4-tetrahydronaphth-2-yl)-2,3-dihydro-2-thioxo-1H-imidazole hydrochloride at lowering norepinephrine levels (FIG. 57), and increasing dopamine and the dopamine/norepinephrine ratio (FIGS. 56 and 58).

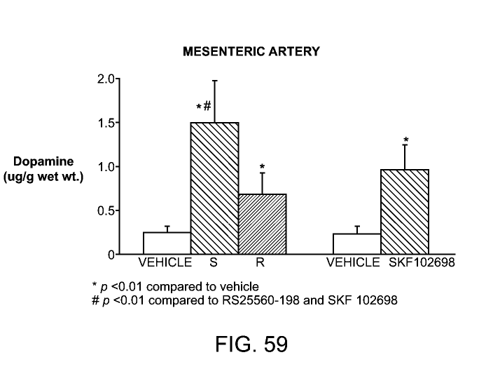

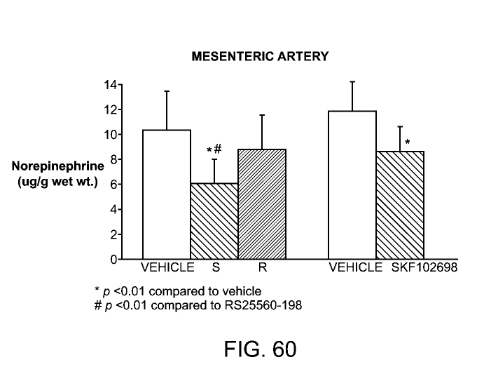

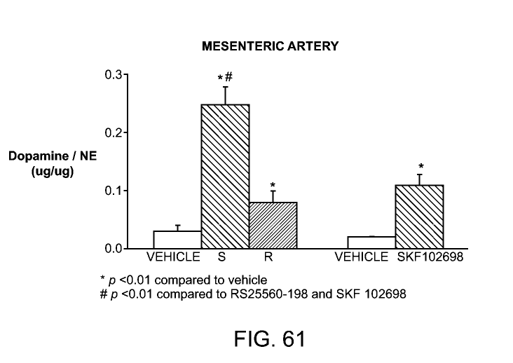

In the mesenteric artery, relative to treatment with vehicle, (R)-5-Aminomethyl-1-(5,7-difluoro-1,2,3,4-tetrahydronaphth-2-yl)-2,3-dihydro-2-thioxo-1H-imidazole hydrochloride resulted in a significant increase in dopamine (FIG. 59) and the dopamine/norepinephrine ratio (FIG. 61) (p<0.01), but did not significantly lower norepinephrine levels (FIG. 60). nepicastat was significantly more effective (p<0.01) than (R)-5-Aminomethyl-1-(5,7-difluoro-1,2,3,4-tetrahydronaphth-2-yl)-2,3-dihydro-2-thioxo-1H-imidazole hydrochloride at lowering norepinephrine levels (FIG. 60), and increasing dopamine and the dopamine/norepinephrine ratio (FIGS. 59 and 61).

Comparing nepicastat with SKF102698 at 30 mg/kg) in the cerebral cortex, dopamine concentration in the cortex was significantly greater (p<0.01) than vehicle for SKF 102698 at a dose of 30 mg/kg (FIG. 53). The increase above vehicle was greater for SKF102698 than for nepicastat (p<0.01). Norepinephrine concentration was significantly lower than vehicle for SKF 102698, and the decrease was greater for SKF 102698 than for nepicastat (p<0.01) (FIG. 44). The dopamine/norepinephrine ratios in the cortex were significantly (p<0.01) greater than vehicle for SKF 102698 (FIG. 55), and the increase above vehicle was greater for SKF 102698 than for nepicastat (p<0.01).

The dopamine concentration in the left ventricle was significantly greater (p<0.01) than vehicle for SKF102698 (FIG. 56), and the increase above vehicle was greater for nepicastat than for SKF 102698 (p<0.01). Norepinephrine concentration was not different from vehicle with SKF 102698 treatment, however treatment with nepicastat significantly lowered norepinephrine relative to vehicle more than SKF 102698 (p<0.01) (FIG. 57). The dopamine/norepinephrine ratios in the left ventricle were significantly (p<0.05) greater than vehicle for SKF102698 (FIG. 58), and the increase above vehicle was greater for nepicastat than for SKF 102698 (p<0.05).

The dopamine concentration in the mesenteric artery was significantly greater than vehicle for SKF102698 (FIG. 59), and the increase above vehicle was greater for NEPICASTAT than for SKF102698. Norepinephrine concentration was significantly lower than vehicle with SKF 102698 treatment, and treatment with nepicastat significantly lowered norepinephrine relative to vehicle more than SKF102698(FIG. 60). The dopaminelnorepinephrine ratios in the left ventricle were significantly greater than vehicle than for SKF 102698 (FIG. 61), and the increase above vehicle was greater for nepicastat than for SKF102698.

In conclusion, the data show that nepicastat is a potent inhibitor of DBH in vivo in the mesenteric artery, left ventricle, and cerebral cortex of SHR six hours after the third of three oral doses administered 12 hours apart. The S enantiomer, nepicastat was more potent than the R enantiomer ((R)-5-Aminomethyl-1-(5,7-difluoro-1,2,3,4-tetrahydronaphth-2-yl)-2,3-dihydro-2-thioxo-1H-imidazole hydrochloride) in all three tissues at 30 mg/kg. Furthermore, nepicastat was more effective than SKF 102698 in the mesenteric artery and left ventricle, but less effective in the cerebral cortex, following three doses at 30 mg/kg administered over 24 hours.

### Example 13

Nepicastat was prepared and administered as the free base equivalent. Nepicastat and methimazole were dissolved in vehicle (66.7% propylene glycol:33.3% dH₂O) to yield dosing solutions of appropriate concentrations so that all doses could be administered in a 1.0 ml/kg volume.

Male Sprague-Dawley rats, weighing 180-200 grams, were fed an iodine deficient diet (Purina, 5891C, Lot 1478, 0.066±0.042 mg iodine/kg sample) ad libitum 14 days prior to treatment. Animals were weighed and randomly assigned to one of the following treatment groups (n=12 per group): Nepicastat at 2.0 mg/kg, Nepicastat at 6.2 mg/kg, R, Methimazole at 1 mg/kg, or vehicle at 1 ml/kg. Each group of rats was dosed orally in the evening and the following morning, approximately 12 hours apart, for 10 consecutive days.

At four hours after the second dose, on day 10, rats were anesthetized with halothane, decapitated, and the cortex, striatum, and mesenteric artery were harvested and weighed. Tissue samples were not harvested from the methimazole groups as they only served as positive controls for determination of thyroid function. The mesenteric artery, cortex, and striatum were immediately placed in 0.4M iced perchloric acid and analyzed for norepinephrine and dopamine levels the same day using HPLC.

Orbital blood samples were taken at day—3, 0, 3, 7, and 9 (day 0 was the first day of dosing). Serum samples were analyzed for T₃ and T₄ levels using a radioimmunoassay.

To statistically evaluate changes in T₃ and T₄ levels, a change from baseline was calculated from the day-3 time point. A non-parametric two-way within subject analysis of variance (ANOVA) was conducted. Also a one-way ANOVA was performed to detect if a significant difference from control occurred. Pairwise analyses between controls and each treatment group were carried out using Fisher's LSD strategy to control the experiment-wise error rate. For statistical analysis of catecholamine levels, a one-way ANOVA with factor DOSE was performed. Pairwise analyses between treated and controls at each dose were carried out using Fisher's LSD strategy to control the experiment-wise error rate.

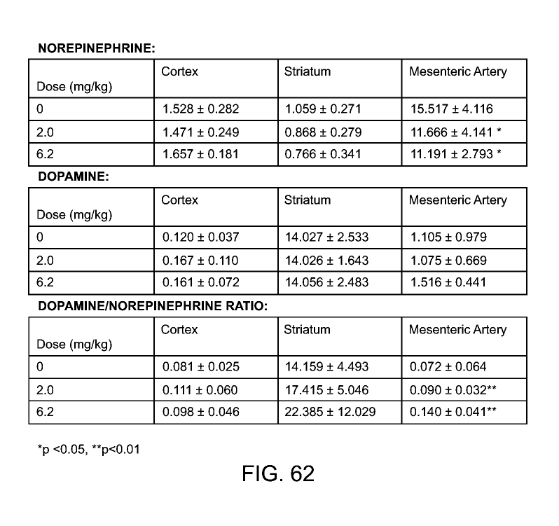

Norepinephrine levels in the nepicastat treated animals were not significantly (p>0.05) different in the cortex compared to vehicle control at doses of 2.0 and 6.2 mg/kg. Norepinephrine levels in the mesenteric artery were significantly (p<0.05) lower at the 2.0 and 6.2 mg/kg dose groups, and norepinephrine levels in the striatum were marginally (p<0.10) lower in both the 2.0 and 6.2 mg/kg dose groups, compared to vehicle control (FIG. 62).

Dopamine levels in all three tissues were not significantly (p>0.05) different from vehicle control at either the 2.0 or 6.2 mg/kg dose group of nepicastat (FIG. 62).

The dopamine/norepinephrine ratio of the cortex and striatum at 2.0 and 6.2 mg/kg RS-25560-197 were not significantly (p>0.05) different from vehicle control, while the ratio of the mesenteric artery at both 2.0 and 6.2 mg/kg nepicastat were significantly (p<0.05) higher than vehicle control (FIG. 62).

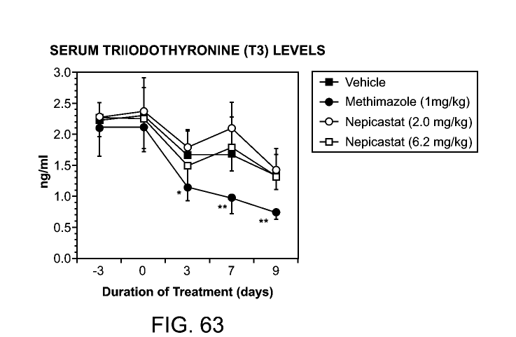

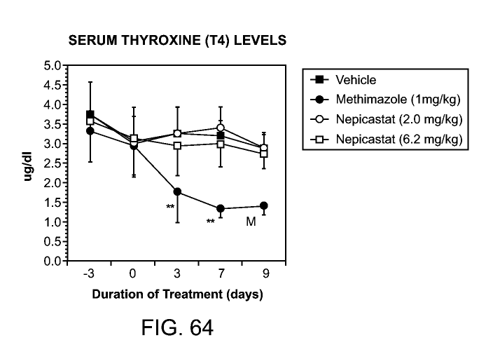

Neither 2.0 or 6.2 mg/kg nepicastat affected thyroid function by altering free T₃ or total T₄ levels in the rat serum. A dose of 1.0 mg/kg of Methimazole, the positive control, significantly (p<0.05) lowered T₃ levels on all treatment days (FIG. 63) and T₄ levels at day 3 and 7 (FIG. 64), compared to vehicle control. T₄ levels of the methimazole treated animals were only marginally (p<0.10) lower on day nine.

nepicastat (2.0 or 6.2 mg/kg) did not cause any significant (p>0.05) changes in dopamine or the norepinephrine levels, or dopamine/norepinephrine ratio when compared to vehicle. In the striatum, a marginally significant (p<0.10) decrease in norepinephrine level was observed in the 6.2 mg/kg dose group, but no other significant changes were observed. In the mesenteric artery, both 2.0 and 6.2 mg/kg of nepicastat produced significantly (p<0.05) lower norepinephrine levels and significantly (p<0.05) higher dopamine/norepinephrine ratios, compared to vehicle, with no significant changes observed in dopamine levels. Thus nepicastat appears to be an effective inhibitor of dopamine β-hydroxylase in vivo, with greater effect in the mesenteric artery than the cerebral cortex or striatum following 10 days of dosing in Sprague-Dawley rats.

### Example 14

This study was performed to determine the dopamine and norepinephrine concentrations in kidney medulla and kidney cortex from dogs dosed with nepicastat. Adult male beagle dogs were randomly assigned to four groups of 8 dogs per group and dosed by oral administration with nepicastat. Nepicastat was delivered in doses of 5, 15 and 30 mg/kg placed in single capsules. Vehicle was an empty capsule. Each dog received 2 doses daily, morning and afternoon (8-10 hours apart) for four days. On the fifth day, each dog received a single dose in the morning and the dogs were euthanized six hours after the last dose. Samples of kidney medulla and kidney cortex were rapidly harvested, weighed, placed in cold 0.4 M perchloric acid, frozen in liquid nitrogen and stored at −70° C.

To quantitate concentrations of norepinephrine (NE) and dopamine (D), each tissue was homogenized by brief sonication in 0.4 M perchloric acid. After sonication, the homogenates were centrifuged at 13,000 rpm in a microfuge for 30 minutes at 4° C. An aliquot of each supernatant was removed and spiked with 3,4-dihydroxybenzylamine (DHBA) as internal standard. The extract from each sample was subjected to HPLC separation using electrochemical detection. The method has a quantitation limit of 2.0 ng/mL and a linear range of 2.0 ng/mL to 400 ng/mL for each analyte.

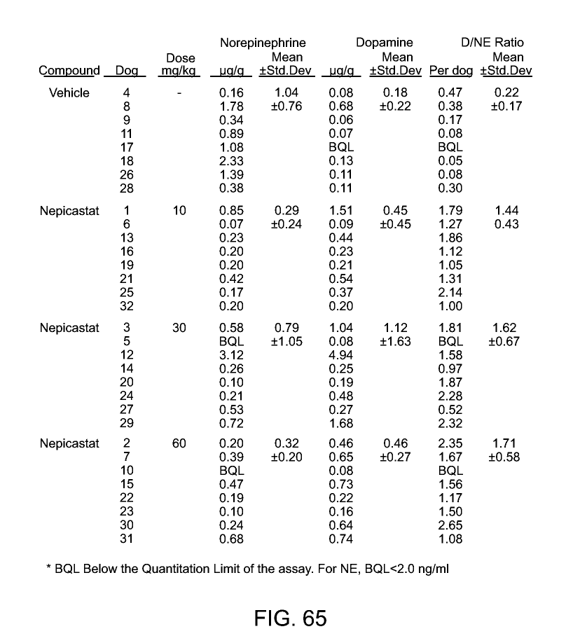

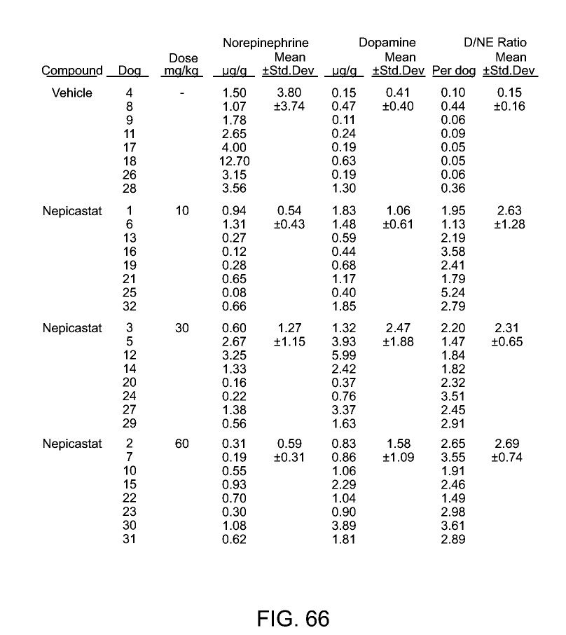

The analytical results are presented in FIGS. 65 and 66. Each analyte determination was normalized to the weight of the tissue sample and expressed as μg of analyte per gram of tissue. The table contains concentrations of dopamine, norepinephrine and the ratio of dopamine concentration to norepinephrine concentration (D/NE) obtained for each dog. In addition, the calculated means and standard deviations for each analyte and D/NE ratio are provided for each treatment group.

### Example 15

Male Beagle dogs (Marshall farms, North Rose, N.Y.) weighing between 9-16 kg were used in the study. The animals were allowed water ad libitum and given food once daily at ˜10.00 AM. Animals were randomly assigned to one of the following treatment groups (n=8/group): placebo (empty capsule), or nepicastat at 2 mg/kg b.i.d (4 mg/kg/day). Each animal received 2 doses daily, morning and afternoon (8-10 hours apart). Daily blood samples (10 ml) were drawn 6 h after the AM dose for measurement of plasma levels of nepicastat and catecholamines. The blood was collected in tubes containing heparin and glutathione and centrifuged at −4° C. within 1 h of collection. The plasma was separated and divided into two samples, one for the measurement of plasma catecholamines and the other for analysis of nepicastat.

Tissue samples were also taken from the dogs at the end of the study in case it was deemed necessary to analyse tissue catecholamines at a later point. On day 15, 6 hours after the AM dose, a final blood sample (10 ml) was taken. Dogs were anesthetized with sodium pentobarbital (40 mg/kg, iv), placed on a necropsy table and euthanized with a second injection of pentobarbital (80 mg/kg,iv). A rapid bilateral transthoracotomy and abdominal incision was performed. Biopsies were taken from the renal artery and left ventricle. The skull was opened to expose the frontal lobe of the cerebral cortex and a biopsy was taken. Tissue samples were weighed, placed on iced 0.4 M perchloric acid, frozen in liquid nitrogen and stored at −70° C. until analyzed.

Plasma NE, DA and EPI were anaylysed by HPLC using electrochemical detection. Plasma concentration of nepicastat was determined by HPLC using electrochemical detection.

The Box-Cox transformations indicated that the logarithm was an appropriate variance stabilizing transformation; hence all analyses were performed on the log-values. The BQL (below quantitation limit) in the DA concentration of dog 1 at day 10 was set to 0; ln (0) was set to missing. The analysis was performed using a mixed model (using PROC MIXED) with the day and treatment categorical variables being fixed and the dog within treatment being a random factor. For the fixed effects, the interaction between the day and the treatment was included, since the difference between the drug and placebo groups varies from day to day. Contrasts were calculated using the CONTRAST statement, which correctly takes into account the error terms for each particular contrast. In particular, the contrasts comparing the treatment group to the drug group uses the dog mean square for its error term, while the comparisons used to establish steady state are all within dog comparisons, and require the error mean square.

The time period of steady state was calculated using the Helmert transformation (cf. SAS PROC GLM manual). These transformations compare each treatment mean with the average of the treatment means of the time points following. The steady state period is defined to start at the first time point following the maximum time at which the Helmert contrast is statistically significant. However, since this method can fail to detect a smoothly changing process, as appears might be the case here, the slope of the analyte concentration during the steady state period also was calculated. The slope during the steady state period was calculated for each dog individually, yielding one slope per animal. Univariate statistics on the slopes were then calculated, with Normal theory confidence intervals built on the mean slope, and the hypothesis of slope equalling zero was tested, and its Normal theory p-value was calculated. This slope analysis was used as the basis for determining whether the steady state period was a period of changing concentration.

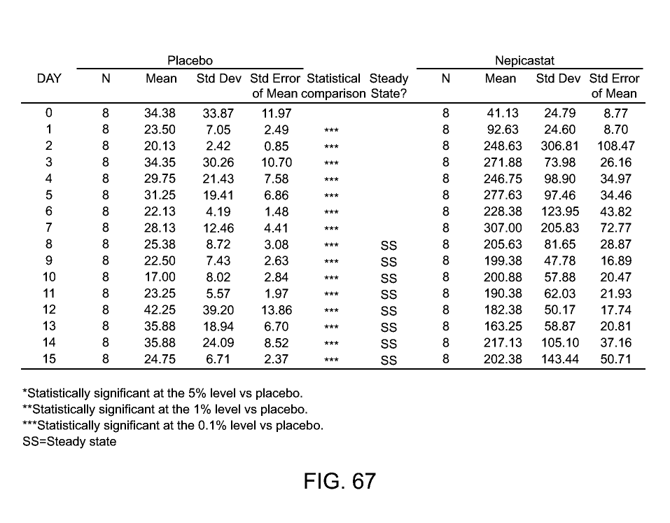

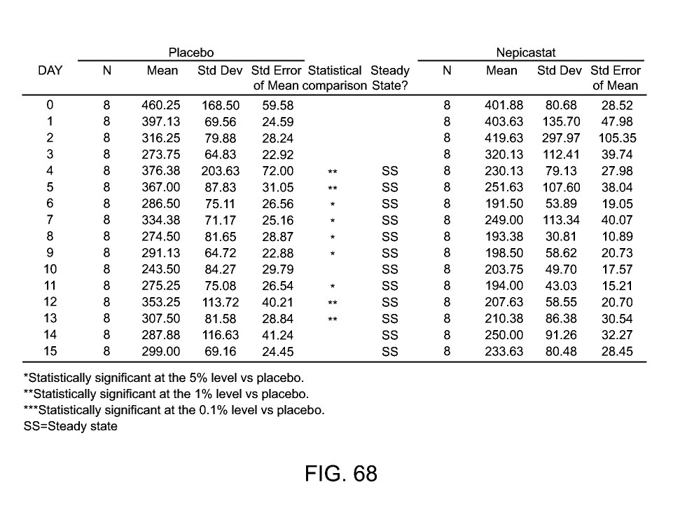

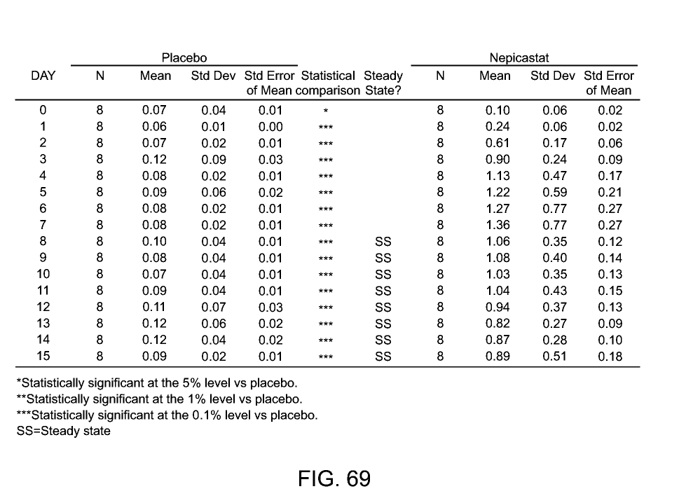

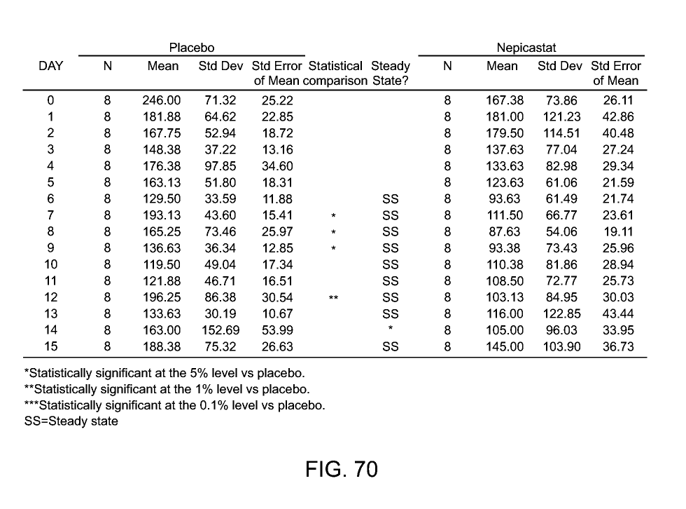

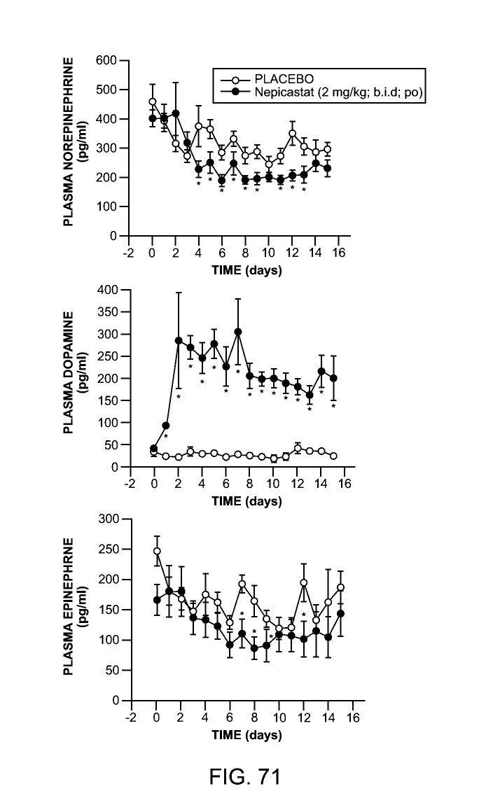

When compared to the placebo group, nepicastat (2 mg/kg, b i d) produced significant decreases in plasma NE (2.1 fold) and EPI (1.91 fold) and significant increases in plasma DA (7.5 fold) and DA/NE ratio (13.6 fold) (see FIG. 67-71). The peak decreases in plasma NE and EPI were observed at day 6 and day 8, respectively, whereas the peak increases in plasma DA and DA/NE ratio were observed at day 7 and day 6, respectively. The effects on plasma NE, DA and EPI attained steady-state at approximately 4, 8 and 6 days post-dose, respectively. The changes in plasma DA and DA/NE ratio were significantly different from placebo on all days post-dose. The changes in plasma NE were significantly different from placebo on days 4-9 and days 11-13 post dose. The changes in plasma EPI were significantly different from placebo on days 7-9 and day 12 post-dose.

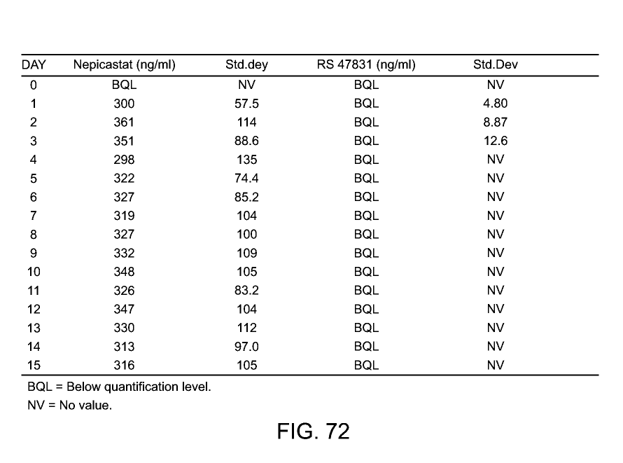

Administration of nepicastat (2 mg/kg, bid) produced significant plasma levels of the drug on all days (FIG. 72). The peak levels were observed at 2 days post-dose. No significant levels of the N-acetyl metabolite of nepicastat were detected on any of the days.

Chronic (14.5 days) administration of nepicastat (2 mg/kg, bid, po) produced significant decreases in plasma NE and EPI and significant increases in plasma DA and DA/NE ratio. These changes reflect inhibition of the sympatho-adrenal system via inhibition of the enzyme dopamine-β-hydroxylase.

### Example 16

Nepicastat was weighed and put into capsules (size 13—Torpac; East Hanover, N.J.) to yield doses of 5, 15, and 30 mg/kg per capsule (given b.i.d. to yield doses of 10, 30 and 60 mg/kg/day). The initial dog weight was used to determine the dose for each animal. Dogs receiving 0 mg/kg/day received empty capsules (placebo). All doses of nepicastat were administered as free base equivalents.

Thirty-two male beagle dogs, weighing 10-12 kg, were randomly assigned to one of the following 4 treatment groups (n=8 per group): nepicastat at 0 mg/kg/day (placebo), 10 mg/kg/day (5 mg/kg b.i.d.), 30 mg/kg (15 mg/kg b.i.d.), or 60 mg/kg/day (30 mg/kg b.i.d.). Dog numbers 1-16 were assigned as dose group A and dog numbers 17-32 as dose group B. The terminal surgery for tissue harvest was performed over 2 days with 16 animals studied per day. Two or 3 days before the first compound administration each dog was weighed and skin regions overlying both cephalic, saphenous and jugular veins were shaved. Dosing consisted of oral administration of one capsule with the second given 8-10 hr later. Dogs were dosed as scheduled on days 1-3. On day 4, prior to the AM dose, 3 ml of blood were obtained from a jugular vein for determination of baseline plasma compound levels. The dog was then administered the AM dose, and at 1, 2, 4 and 8 hr following the dose additional 3 ml blood samples were collected for determination of plasma compound levels. Blood samples were put into tubes containing heparin, centrifuged at 4° C. and stored at −20° C. until analysis. The PM dose was then administered as scheduled. The AM dose was administered as scheduled on the days of surgery. Approximately 6 hr after the AM dose, a final 3 ml blood sample was taken from the jugular vein for determination of plasma compound levels. The dog was then anesthetized with pentobarbital Na (˜40 mg/kg), given iv in a cephalic or saphenous vein, and delivered to the necropsy room where an additional dose of pentobarbital Na was given (˜80 mg/kg, iv). The left ventricle, renal artery, kidney, renal medulla, renal cortex and cerebral cortex were then rapidly harvested, weighed, put into 2 ml iced 0.4M perchloric acid, frozen in liquid nitrogen and stored at −70° C. until analysis for catecholamines by HPLC using electrochemical detection. All tissue samples were divided into 2 portions, the second of which were immediately frozen in liquid nitrogen and stored at −70° C. for determination of tissue compound levels. A third transmural sample taken from the left ventricle was immediately frozen in liquid nitrogen and stored at −70° C. for use in receptor binding studies.

Ventricles were homogenized in 50 mM Tris-HCl, 5 mM Na₂EDTA buffer (pH 7.4 at 4° C.) using a Polytron P-10 tissue disrupter (setting 10, 2×15 second bursts). Homogenates were centrifuged at 500×g for 10 minutes and the supernatants stored on ice. The pellets were washed by resuspension and centrifugation at 500×g and the supernatants combined. The combined supernatants were centrifuged at 48,000×g for 20 minutes. The pellets were washed by resuspension and centrifugation once in homogenizing buffer and twice in 50 mM Tris-HCl, 0.5 mM EDTA buffer (pH 7.4 at 4° C.). Membranes were stored at −70° C. until required. Saturation experiments were conducted using [³H] CGP-12177 in buffer containing 50 mM Tris-HCl, 0.5 mM EDTA (pH 7.4 at 32° C.). Non-specific binding was defined by 10 uM isoproterenol. Total bound, non-specific bound and total count tubes were set up for eight concentrations of [³H] CGP-12177 ranging from 0.016 nM to 2 nM. Samples wee incubated at 32° C. for 60 minutes. Samples were filtered over 0.1% PEI pre-treated GF/B glass fiber filtermats using a Brandel cell harvester. Samples wee washed with room temperature water three times for 3 seconds. Aquasol scintillation fluid was added to each vial and radioactivity determined by liquid scintillation counting. Saturation binding isotherms were analyzed after first converting total ligand concentrations to free ligand concentrations (total−bound=free). Individual saturation isotherms were completed for each tissue. Membranes were assayed for protein using the Bio-Rad protein binding method and using gamma globulin as the standard. Receptor densities were expressed, per mg protein, as mean for each treatment group. Tissue catecholamine levels were analyzed by comparing nepicastat-treated groups with the placebo (control) treated groups. A nonparametric one-way analysis-of-variance (ANOVA) with factor DOSE was performed for each tissue and each catecholamine measure separately. Pairwise analyses between treated and controls at each dose were carried out using Dunnett's test to control the experiment-wise error rate. Student-Neuman-Kuels and Fisher's LSD tests were performed as validation. Analysis of tissue and plasma compound levels were performed in 2 ways. First, individual t-tests were run to compare each dose level to a factored level of its partner dose for each parameter. For example, three times the level of compound present at 10 mg/kg in a particular tissue or plasma should be comparable to the compound level observed in the 30 mg/kg group. Additionally, a linear orthogonal contrast was calculated for all three doses within the context of a one-way ANOVA. A paired t-test was used to determine any differences in binding between the vehicle treated group and the mg/kg/day nepicastat group.

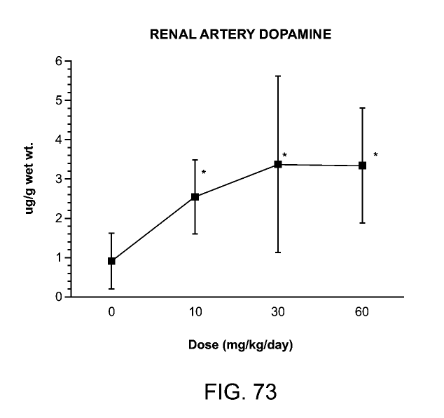

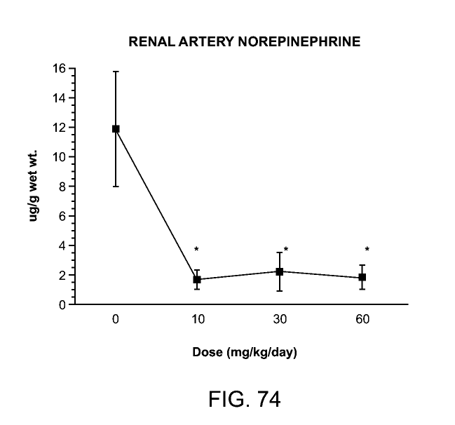

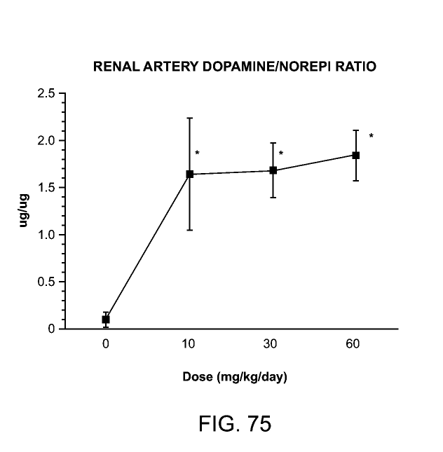

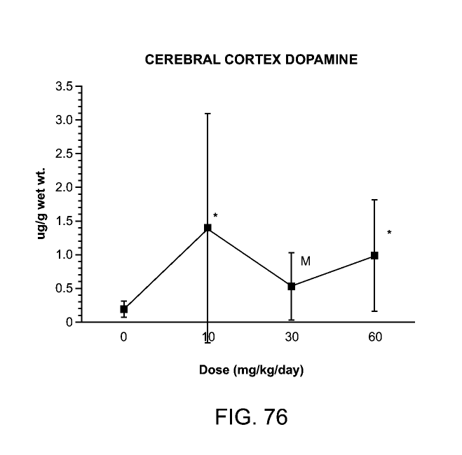

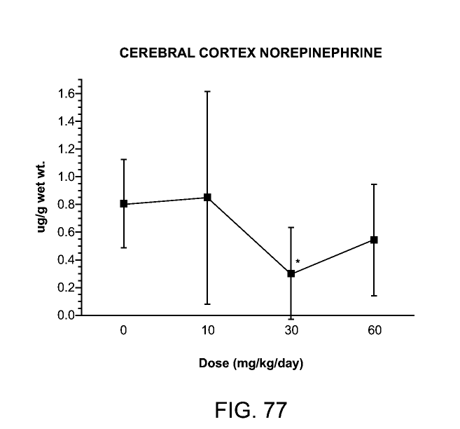

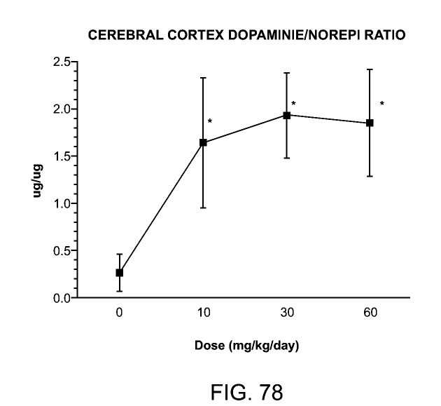

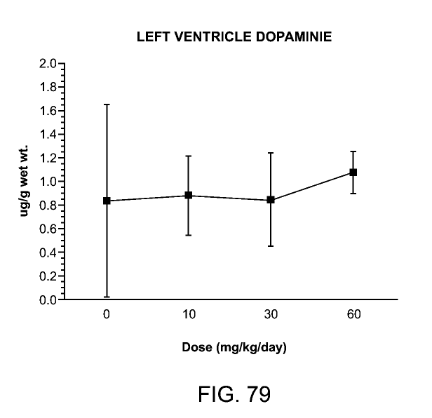

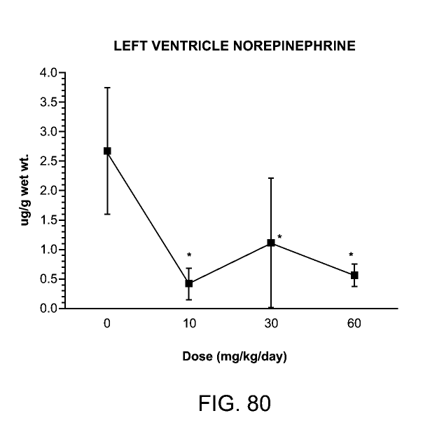

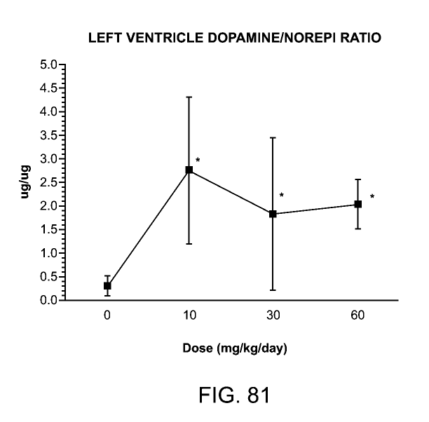

In the renal artery, nepicastat administered at doses of 10, 30 and 60 mg/kg/day significantly (p<0.01) decreased norepinephrine levels by 86%, 81% and 85%, respectively (FIG. 73). Dopamine levels were significantly (p<0.01) increased at doses of 10, 30 and 60 mg/kg/day by 180%, 273% and 268%, respectively (FIG. 74). Doses of 10, 30 and 60 mg/kg/day nepicastat significantly (p<0.01) increased the dopamine/norepinephrine ratio by 1711%, 1767% and 1944%, respectively, compared to placebo (FIG. 75). Following administration of 10 and 60 mg/kg/day nepicastat, dopamine levels were significantly (p<0.01) increased 632% and 411%, respectively in the cerebral cortex (FIG. 76). The dopamine/norepinephrine ratio was significantly (p<0.01) increased 531% after 10 mg/kg/day nepicastat and 612% following administration of 60 mg/kg/day nepicastat (FIG. 78). Norepinephrine levels were not significantly (p>0.01) affected at these 2 doses (FIG. 77). At 30 mg/kg/day, norepinephrine was significantly (p<0.01) reduced by 63% and the ratio significantly (p<0.01) elevated by 86%, while dopamine levels marginally (0.05<p<0.10) increased 174%, compared to placebo (FIGS. 76-78). Following administration of 10, 30 and 60 mg/kg/day nepicastat, norepinephrine levels were significantly (p<0.01) decreased by 85%, 58% and 79%, respectively in the left ventricle (FIG. 80). The dopamine/norepinephrine ratio significantly (p<0.01) increased 852%, 279% and 607%, respectively, compared to placebo animals (FIG. 81). No significant changes were observed in dopamine levels at doses of 10, 30, and 60 mg/kg/day nepicastat (FIG. 79).

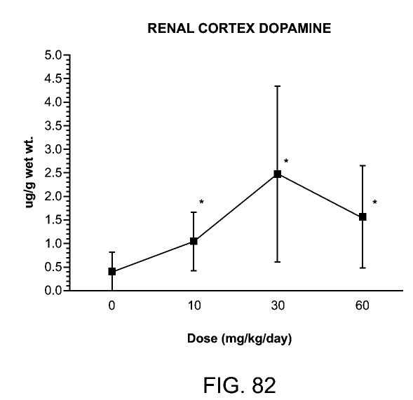

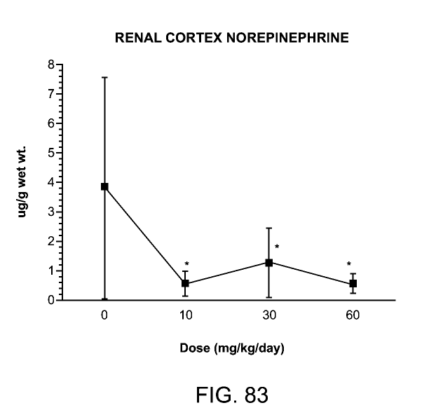

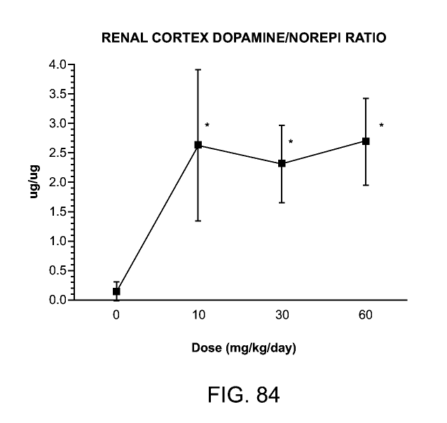

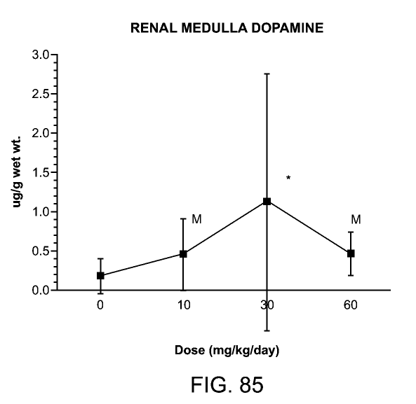

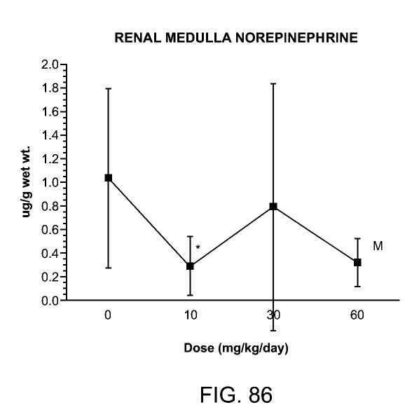

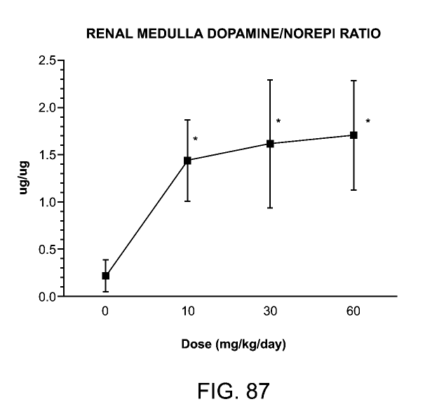

In the renal cortex, compared to placebo, norepinephrine levels were significantly decreased (p<0.01) by 86%, 66% and 85%, respectively, following doses of 10, 30 and 60 mg/kg/day nepicastat (FIG. 83). Dopamine levels were significantly (p<0.01) increased 156%, 502% and 208%, respectively, at these doses (FIG. 82). The dopamine/norepinephrine ratio significantly (p<0.01) increased by 1653%, 1440% and 1693%, respectively, at doses of 10, 30, and 60 mg/kg/day (FIG. 84). In the renal medulla, the dopamine/norepinephrine ratios were significantly (p<0.01) increased by 555%, 636% and 677%, respectively, at doses of 10, 30 and 60 mg/kg/day nepicastat, compared to placebo (FIG. 87). Dopamine levels were significantly (p<0.01) increased 522% at 30 mg/kg/day and marginally (0.05<p<0.10) increased by 150% and 156%, respectively, at 10 and 60 mg/kg/day (FIG. 85). Norepinephrine levels were significantly (p<0.01) decreased 72% following administration of 10 mg/kg/day nepicastat, compared to placebo, and marginally (0.05<p<0.10) decreased by 69% following 60 mg/kg/day (FIG. 86).

Statistical analysis indicated that the concentration of nepicastat in plasma obtained on Day 4 and tissue and plasma obtained on Day 5 was dose-proportional between each dose level and factored levels of its partner dose. Therefore, dose points were determined to be linear, with the following exceptions (a significant result would suggest the data are not linear):

Kidney medulla: 3×10 <30 (p<0.05)

Kidney medulla: 6×10 <60 (p=0.077)

Plasma (day 4): 2×30>60 (p=0.076)

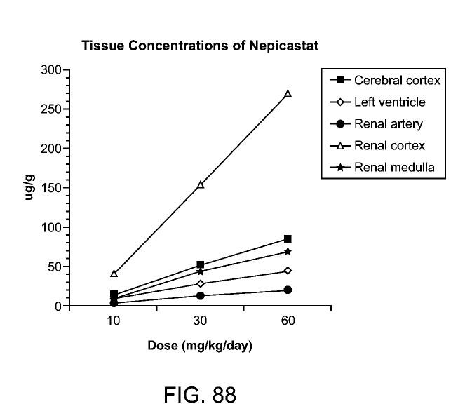

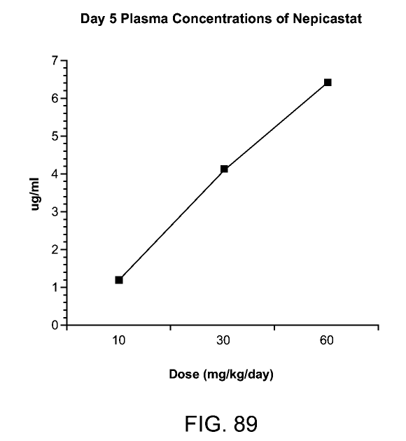

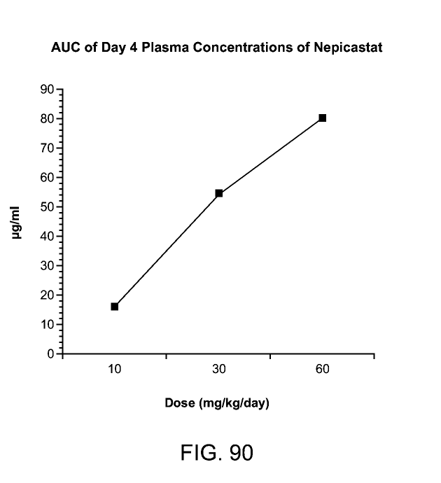

On Day 5, levels of nepicastat in all tissues examined were higher than those in plasma (FIGS. 88-90).

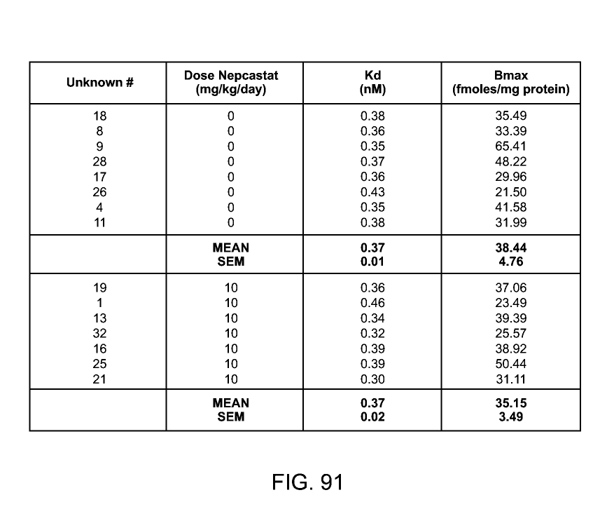

The results demonstrated no difference between left ventricular samples from the 10 mg/kg/day nepicastat treated group and vehicle treated group (FIG. 91).

### Example 17

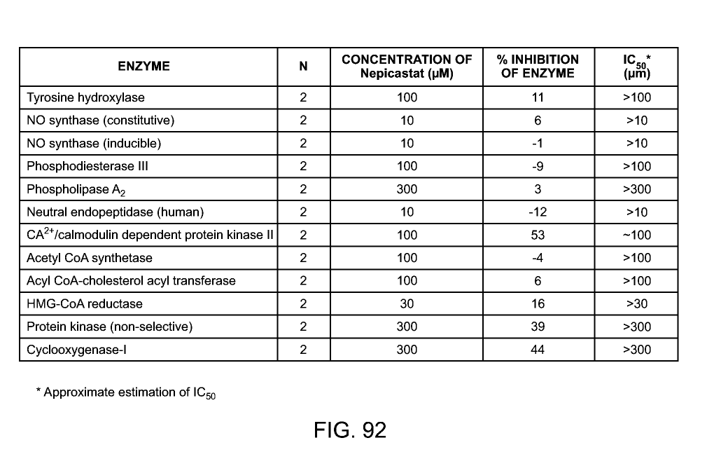

Nepicastat was evaluated for its activity at a range of enzymes including tyrosine hydroxylase, NO synthase, phosphodiesterase III, phospholipase A₂, neutral endopeptidase, Ca²⁺/calmodulin protein kinase II, acetyl CoA synthetase, acyl CoA-cholesterol acyl transferase, HMG-CoA reductase, protein kinase (non-selective) and cyclooxygenase-I. As shown in FIG. 92, nepicastat had an IC₅₀ of >10 μM at all the 12 enzymes studied, therefore is a highly selective (>1000-fold) inhibitor of dopamine-β-hydroxylase.

### Example 18

Bovine DBH from adrenal glands was obtained from Sigma Chemicals (St. Louis, Mo.). Human secretory DBH was purified from the culture medium of the neuroblastoma cell line SK-N-SH and was used to obtain the inhibition data. A lentil lectin-sepharose column containing 25 ml gel was prepared and equilibrated with 50 mM KH₂PO₄, pH 6.5, 0.5 M NaCl. The column was eluted with 35 ml of 10% methyl α, D-mannopyranoside in 50 mM KH₂PO₄, pH 6.5, 0.5 M NaCl at 0.5 ml/min. Fraction containing most enzymatic activities were pooled and concentrated with an Amicon stirred cell using a YM30 membrane. Methyl α, D-mannopyranoside was removed by buffer exchange with in 50 mM KH₂PO₄, pH 6.5, 0.1 M NaCl. The concentrated enzyme solution was aliquoted and stored at −25° C. An HPLC assay was used to measure DBH activity using tyramine and ascorbate as substrates. The method is based on the separation and quantitation of tyramine and octopamine by reverse phase HPLC chromatography (Feilchenfeld, N. B., Richter, H. & Waddell, W. H. (1982). Anal. Biochem: A time-resolved assay of dopamine β-hydroxylase activity utilizating high-pressure liquid chromatography. 122: 124-128.). The assay was performed at pH 5.2 and 37° C. in 0.125 M NaAc, 10 mM fumarate, 0.5˜2.0 μM CuSO₄, 0.1 mg/ml catalase (6,500 u, Boeringer Mannheim, Indianapolis, Ind.), 0.1 mM tyramine, and 4 mM ascorbate. In a typical assay, 0.5-1.0 milli-units of enzyme were added to the reaction mixture and then a substrate mixture containing catalase, tyramine and ascorbate was added to initiate the reaction (final volume 200 μl). Samples were incubated at 37° C. for 30˜40 minutes. The reactions were quenched by the stop solution containing 25 mM EDTA and 240 μM 3-hydroxytyramine (internal standard). The samples (150 μl) were loaded to a Gilson autosampler and analyzed by HPLC using UV detection at 280 nm. PC-1000 software (Thermo Separations products, Fremont, Calif.) was used for integration and data analysis. The HPLC run was carried out at the flow rate of 1 ml/min using a LiChroCART 125-4 RP-18 column and isocratic elution with 10 mM acidic acid, 10 mM 1-heptanesulfonic acid, 12 mM tetrabutylammonium phosphate, and 10% methanol. The remaining percent activity was calculated based on the control without inhibitor, corrected using internal standards and fitted to a nonlinear 4 parameter dose response curve to obtain the IC₅₀ values.

Purification of [¹⁴C]-Tyramine. [¹⁴C]Tyramine hydrochloride was purified by a C18 light load column (two columns combined into one) that was washed with 2 ml of MeOH, 2 ml of 50 mM KH₂PO₄, pH 2.3, 30% acetonitrile, and then 4 ml of 50 mM KH₂PO₄, pH 2.3. A vacuum manifold (Speed Mate 30, from Applied Separations) was used to wash and elute the column by vacuum. After loading of [¹⁴C]tyramine, the column was washed with 6 ml of 50 mM KH₂PO₄, pH 2.3 and eluted with 2 ml of 50 mM KH₂PO₄ containing 30% acetonitrile. The eluate was lyophilized to remove acetonitrile, resuspended in H₂O, and stored at −20° C.

Enzyme Assay by Radioactive Method. Enzymatic activity was assayed using [¹⁴C]tyramine as substrate and a C18 column to separate the product. The assay was performed in 200 ml volume containing 100 mM NaAc, pH 5.2, 10 mM fumaric acid, 0.5 μM CuSO₄, 4 mM ascorbic acid, 0.1 mg/ml catalase and various concentrations of tyramine. The total counts of each reaction was ˜15,000 cpm. Bovine DBH (0.18 ng for each reaction) was mixed with tyramine and inhibitor in the reaction buffer at 37° C. The reaction was initiated by the addition of ascorbate/catalase mixture and was incubated at 37° C. for 30 minutes. The reaction was stopped by the addition of 100 ml of 25 mM EDTA, 50 mM KH₂PO₄, pH 2.3. Entire mixture was loaded to a C18 light load column (two combined into one) that was pre-washed with MeOH and equilibrated with 50 mM KH₂PO₄, pH 2.3. Elution into scintillation vials was carried out with 1 ml of KH₂PO₄, pH 2.3 buffer twice, followed by 2 ml of the same buffer. ReadySafe scintillation fluid (16 ml) was added to the scintillation vials and the samples were counted for ¹⁴C radioactivity.

Nepicastat concentrations of 0, 1, 2, 4, 8 nM were used to study inhibition kinetics at the following tyramine concentrations: 0.5, 1, 2, 3, 4 mM. The ¹⁴C counts were identical in each reaction which was carried out as described above. A blank control without the enzyme was used to obtain the background. The data were corrected for background, converted to activity in nmol/min, and ploted (1/V vs 1/S). Km′ was calculated from the slopes and Y intercepts and linear regression was used to obtain Ki value.

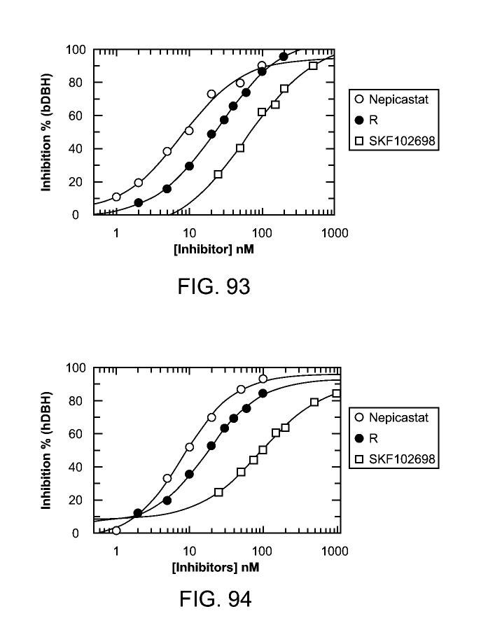

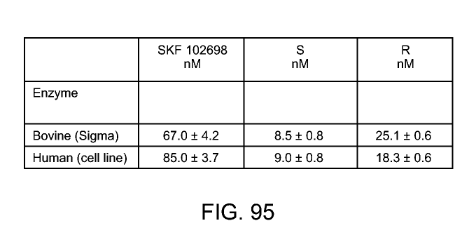

The IC₅₀ values for SKF102698, nepicastat and (R)-5-Aminomethyl-1-(5,7-difluoro-1,2,3,4-tetrahydronaphth-2-yl)-2,3-dihydro-2-thioxo-1H-imidazole hydrochloride against human and bovine DBH were obtained using the HPLC assay at the substrate concentrations of 0.1 mM tyramine, 4 mM ascorbate at pH 5.2 and 37° C. All three compounds caused a dose-dependent inhibition of DBH activity on both bovine and human enzyme (FIGS. 93 and 94).

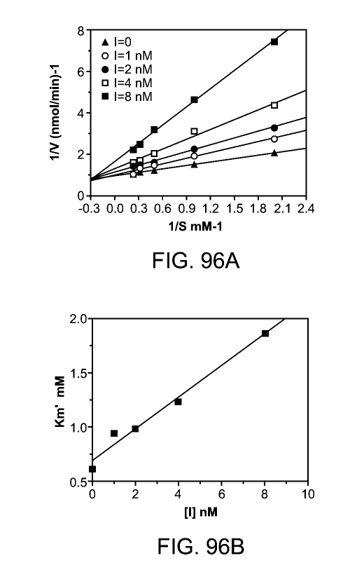

The IC₅₀ values for nepicastat, (R)-5-Aminomethyl-1-(5,7-difluoro-1,2,3,4-tetrahydronaphth-2-yl)-2,3-dihydro-2-thioxo-1H-imidazole hydrochloride and SKF 102698 are given in FIG. 95. The S enantiomer (nepicastat) was more potent than the R enantiomer ((R)-5-Aminomethyl-1-(5,7-difluoro-1,2,3,4-tetrahydronaphth-2-yl)-2,3-dihydro-2-thioxo-1H-imidazole hydrochloride by 3-fold against bovine DBH and 2-fold against the human enzyme. nepicastat was more potent than SKF 102698 by 8-fold against bovine enzyme, and 9-fold against human DBH.

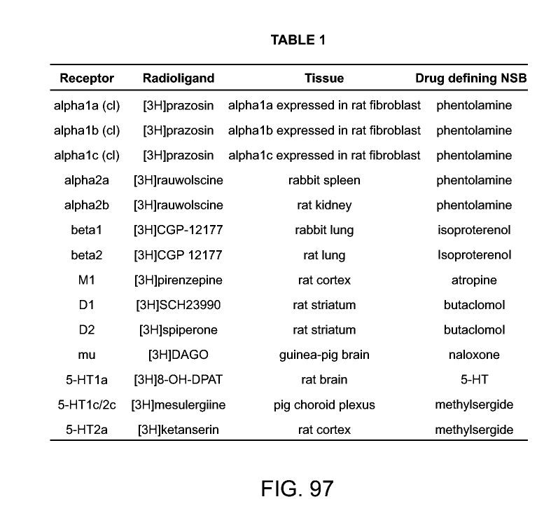

FIG. 96 shows the Lineweaver-Burk plot of the inhibition data against bovine DBH (upper panel) and the plot of apparent Km versus inhibitor concentration (lower panel). A Km of 0.6 mM was determined from the plot. nepicastat (1-8 nM) caused a major shift in Km, as would be predicted for a competitive inhibitor. The inhibition of bovine DBH by nepicastat appears to be competitive with tyramine. A Ki of 4.7±0.4 nM was calculated by linear regression.

Nepicastat was a potent inhibitor of both human and bovine DBH. It was 8-9-fold more potent than SKF102698. nepicastat (the S enantiomer) is 2-3 fold more potent than (R)-5-Aminomethyl-1-(5,7-difluoro-1,2,3,4-tetrahydronaphth-2-yl)-2,3-dihydro-2-thioxo-1H-imidazole hydrochloride (the R enantiomer). The inhibition of bovine DBH by nepicastat appeared to be competitive with tyramine, with a Ki of 4.7±0.4 nM.

### Example 19

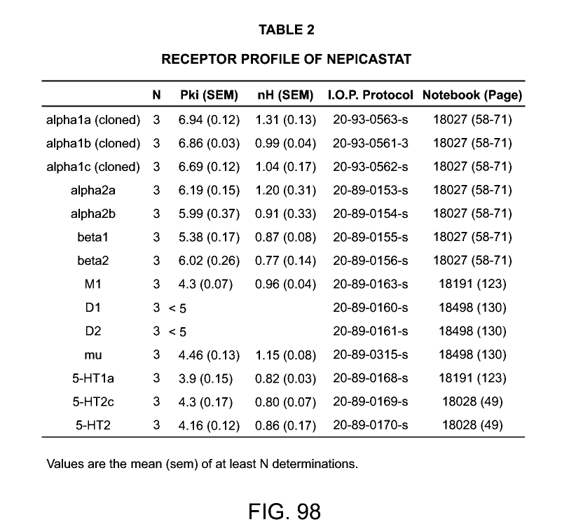

The affinity of nepicastat was determined in the bindings assays outlined in FIG. 97. Standard radioligand filtration binding methods were used.

Competition binding data were analyzed by iterative curve fitting to a four parameter logistic equation. Hill coefficients and IC₅₀ were obtained directly. pKi (−log of Ki) of competing ligands were calculated from IC₅₀ values using the Cheng-Prusoff equation.

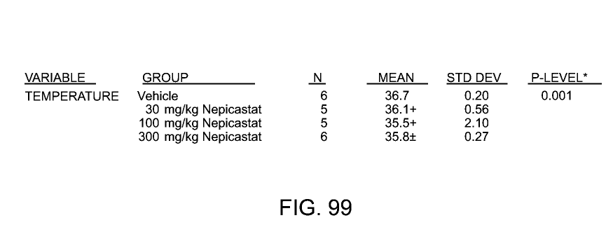

Nepicastat had moderate affinity for alpha₁ receptors (pKi of 6.9-6.7). The affinity at all other receptors examined was relatively low (pKi <6.2) (FIG. 98).

### Example 20

Vehicle and nepicastat monohydrate powder were obtained from the Center for Pharmaceutical Development, Syntex Preclinical Research and Development. At the time of dosing, a 60-mg/ml Nepicastat formulation was prepared by mixing vehicle with Nepicastat powder, followed by shaking. The 6- and 20-mg/ml Nepicastat formulations were prepared by diluting the 60-mg/ml formulation with vehicle. The reconstituted Nepicastat formulations retained potency for the duration of use. The aqueous vehicle andnepicastat formulations contained hydroxypropylmethylcellulose, benzyl alcohol, and polysorbate 80.

Dose selection was based on an acute toxicity study in which mice were administered single oral doses of 250, 1000, or 2500 mg/kg of Nepicastat. Clinical signs of toxicity and death occurred at 1000 and 2500 mg/kg.

A single oral dose of vehicle or Nepicastat formulation was administered by gavage to each mouse using a rodent intubator. The oral route was selected because it is a proposed clinical route of administration. Dose volumes were calculated on the basis of individual body weights recorded before dosing (body weight data are not tabulated in this report). Food and water were withheld from the mice 2.5 to 3.5 hours before dosing, instead of 1.5 hours as specified in the protocol. This deviation did not affect the integrity of the study.

Clinical observations were recorded before dosing. Beginning 60 minutes after dosing, mice in each treatment group were evaluated in groups of up to 3 over an interval of approximately 10 minutes each for clinical observations and protocol-specified behavioral tests.¹ One mouse in the 30-mg/kg group and 1 mouse in the 100-mg/kg group died after dosing and they were removed from the study. Surviving mice were euthanatized and removed from the study at the end of the observation/testing period.

Mice in groups of 6 males each were administered single oral doses of 0 (vehicle), 30, 100, or 300 mg/kg of nepicastat by gavage. Clinical observations and behavioral tests were initiated 60 minutes after administration of the test formulation. At the end of the observation period, all surviving mice were euthanatized and removed from the study.

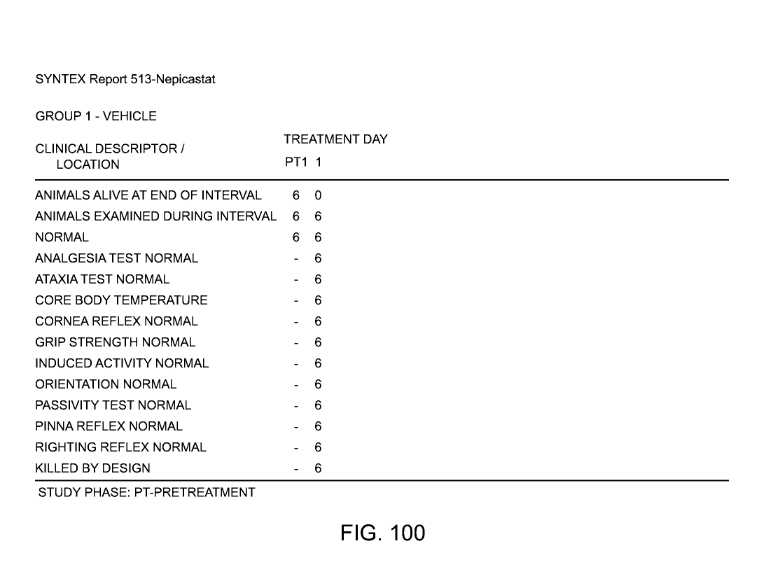

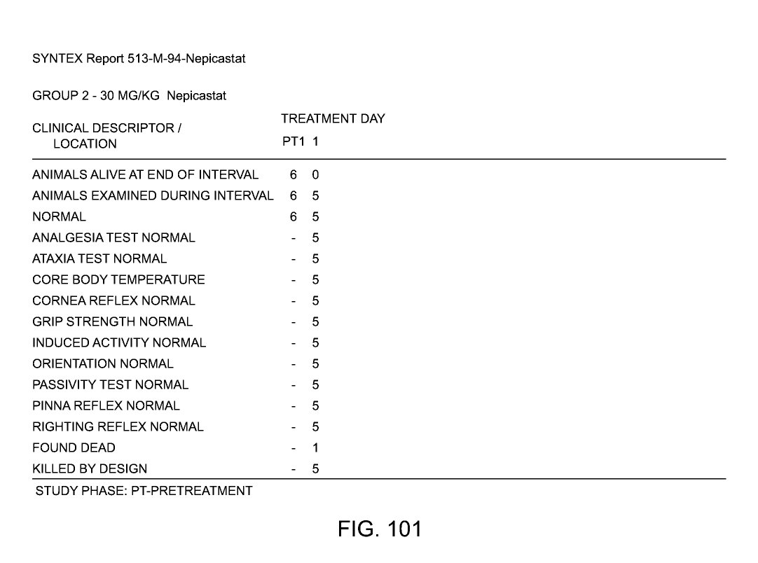

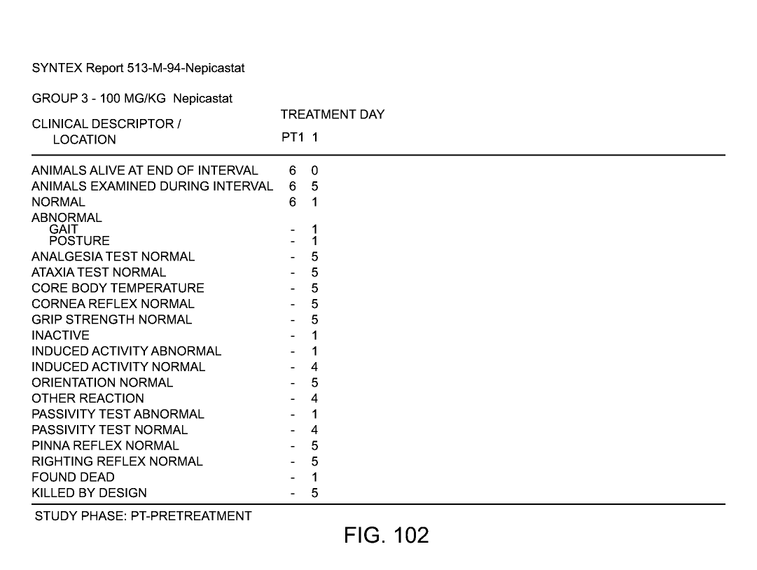

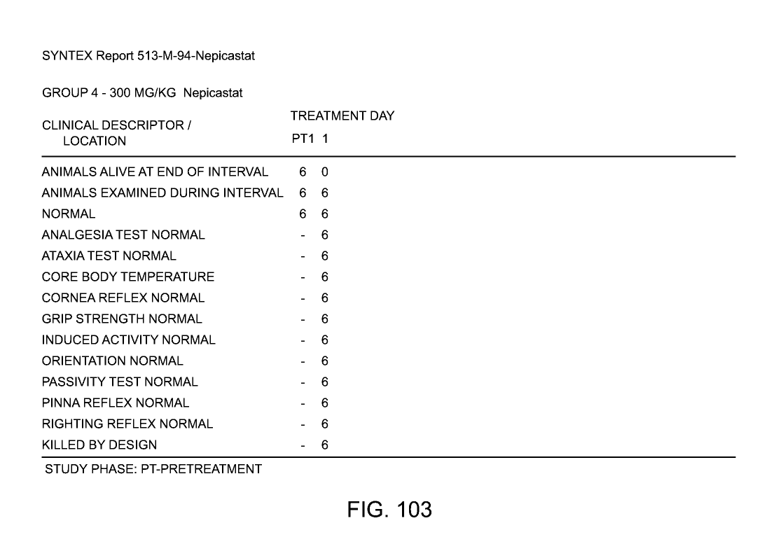

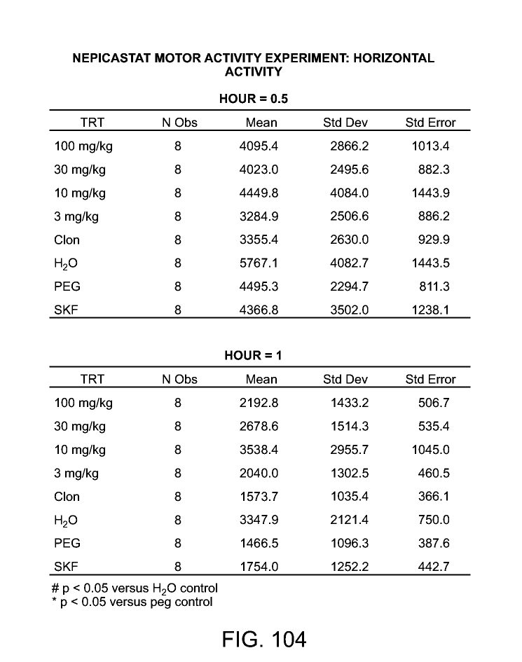

Lower body temperatures were present in the 30-, 100-, and 300-mg/kg groups compared with the vehicle-control group. No treatment-related clinical or gross behavior changes were present. Rectal body temperature data are summarized in FIG. 99; observation and behavioral test data are summarized in FIG. 100. No treatment-related clinical or gross behavioral changes were present. (See FIGS. 101-103) Abnormal social grouping (listed as other reaction) occurred among mice in the 100-mg/kg group, but not the 300-mg/kg group; this finding was considered incidental. Clinical/behavioral changes in 1 mouse in the 100-mg/kg group included inactivity, abnormal gait and posture, decreased induced activity, abnormal passivity, and soft/continuous vocalization; these changes were not attributed to Nepicastat. One mouse each in the 30- and 100-mg/kg group died after dosing; the deaths were considered incidental and the mice were removed from the study.

### Example 21

The purpose of this study is to determine if the DBHIs SKF-102698 and nepicastat produce changes in locomotor activity or acoustic startle reactivity. Changes in these behaviors may therefore reflect activity of these compounds in the central nervous system. Adult male Sprague Dawley rats (250-350 g on study day) were obtained from Charles Rivers Labs. Rats were housed under a normal light/dark cycle with lights on between 0900 Hrs. and 2100 Hrs. Animals were housed in pairs in standard metal wire cages, and food and water were allowed ad libitum.

The locomotor activity boxes consisted of a Plexiglas® box measuring 18″×18″ by 12″ high. Surrounding the Plexiglas® boxes were Omnitech Digiscan Monitors (model # RXXCM 16) which consisted of a one inch ban of photobeams and photosensors numbering 32 per box. The number of photobeam breaks were analyzed by an Omnitech Digiscan Analyzer (model # DCM-8). The animals were tested in an enclosed room with a white noise generator running to mask extraneous noise.

Acoustic startle reactivity tests were conducted in eight SR-Lab (San Diego Instruments, San Diego, Calif.) automated test stations. The rats were placed individually in a Plexiglas® cylinder (10 cm diameter) which is housed in a ventilated sound-attenuating enclosure. Acoustic noise bursts (a broad band noise with a rise time and fall time of 1 msec) was presented via a speaker mounted 30 cm above the animal. A piezoelectric accelerometer transforms the subject's movement into an arbitrary voltage on a scale of 0 to 4095.

Prior to drug administration, each of seventy-two rats was placed in the startle apparatus, and after a 5 minute adaption period they were presented with an acoustic noise burst every 20 seconds for 15 minutes (45 startles total). The average startle was calculated for each rat by taking the mean of startle number 11 through 45 (the first ten startles will be disregarded). Sixty-four of these rats were then placed in one of eight treatment groups such that each group had similar mean startle values. The eight treatment groups were as follows: SKF-102698 (100 mg/kg) and its vehicle (50% water/50% polyethylene glycol), clonidine (40 μg/kg), nepicastat (3, 10, 30 and 100 mg/kg), and their vehicle, dH₂O. Previous work has shown that this matching procedure to be the most appropriate for startle since there is significant variability in startle response between rats, but a high degree of consistency within rats from one day to the next.

Each day after this testing procedure, eight rats (one rat from each of the eight treatment groups) was injected with their assigned drug treatment and immediately placed individually in a motor activity box. The rats motor activity was monitored for four hours. Next, the rats were placed in a transfer cage for fifteen minutes. At the beginning of this fifteen minutes the rat that has been assigned the clonidine treatment will receive another injection of 40 μg/kg. Next, the rats were placed in the startle apparatus, and after a five minute acclimation period they were presented with a 90 dB noise burst every minute for four hours.

To evaluate motor activity, horizontal activity (number of photobeams broken), number of movements, and rest time were measured. Each parameter was analyzed separately. At each time interval (or called sample), a two-way analysis of variance (ANOVA) was performed using the ranked data (nonparametric technique) to test for the treatment effect blocked by day. Pairwise comparisons for 4 RS-treated groups to the vehicle control were also performed using Dunnett's t-test.

To evaluate startle reactivity, for the 200 milliseconds immediately succeeding each startle the average force exerted by each startled rat over the entire 200 milliseconds, and the maximum force, were measured. The mean maximum and average voltages (MAXMEAN and AVGMEAN) were computed for each treatment (TREAT) at each trial (TRIALN), and then these values were plotted against trial number for each treatment. The plots are attached to the report. Trials 1-60 were set to time=1, trials 61-120 to time=2, trials 121-180 to time=3 and trials 181-240 to time=4. The mean maximum and average startle responses was computed within each time and for each treatment. The means were then used in the statistical analysis. The startle responses were analyzed using analysis of covariance. Treatment comparisons within time were of interest to the investigators, but not time effects within treatments. Therefore, the startle responses were analyzed by time. The model included terms for the day the rat was tested (date), baseline startle response, and treatment. Date was a blocking factor and baseline startle response was a covariate. There were three separate models for each of the objectives stated above. The varying doses of nepicastat were compared to vehicle using Dunnett's procedure in order to control for multiple comparisons.

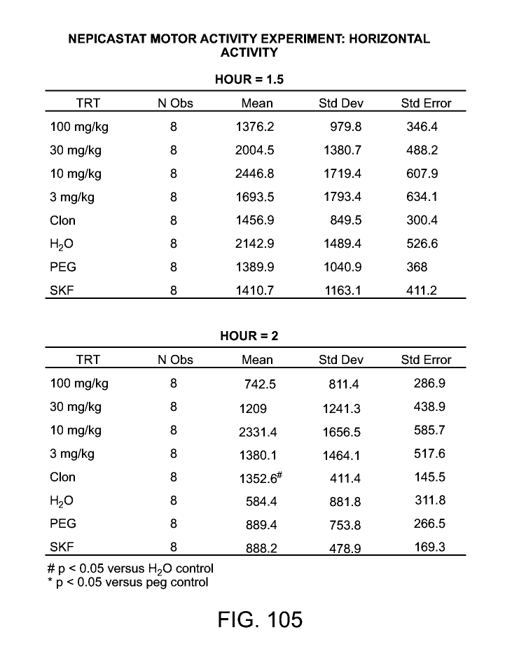

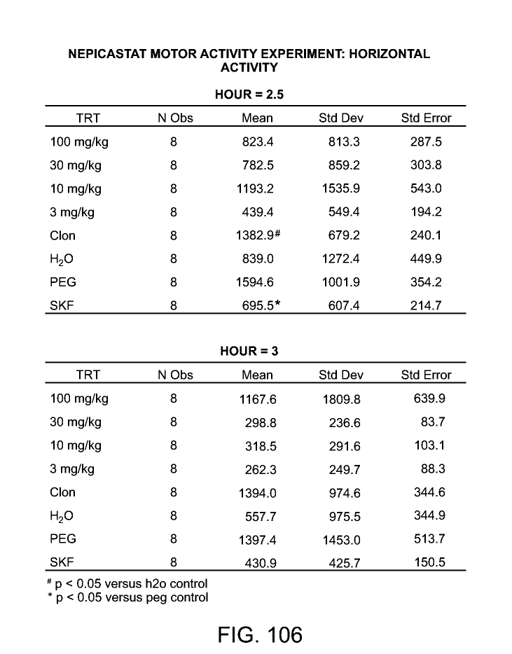

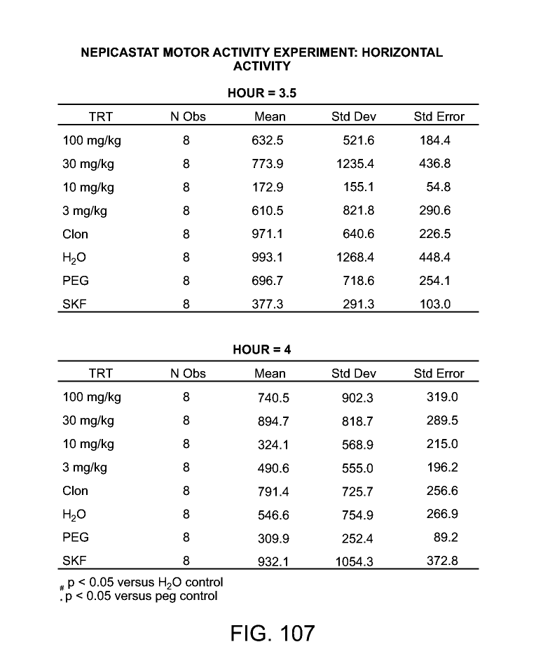

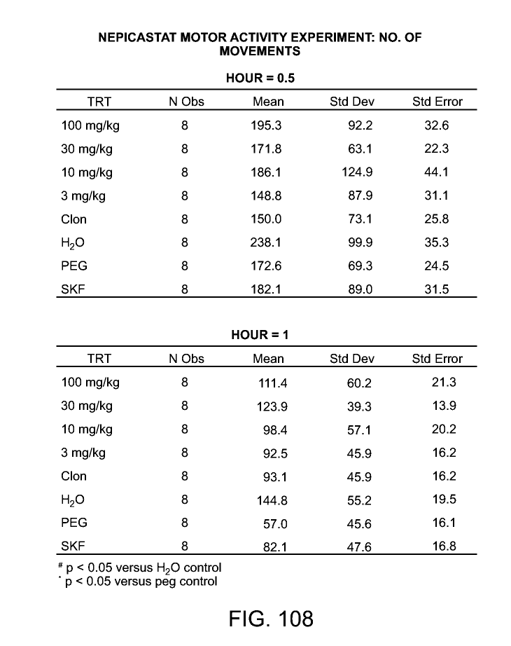

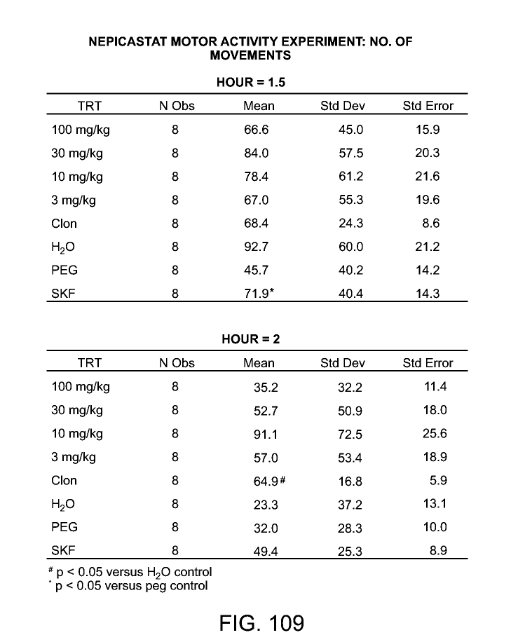

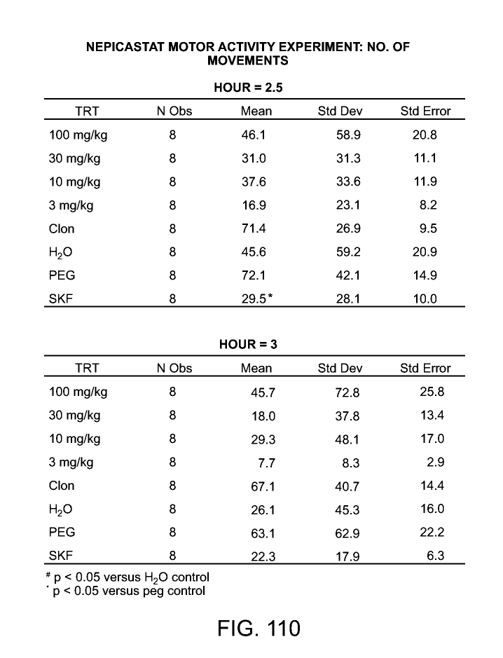

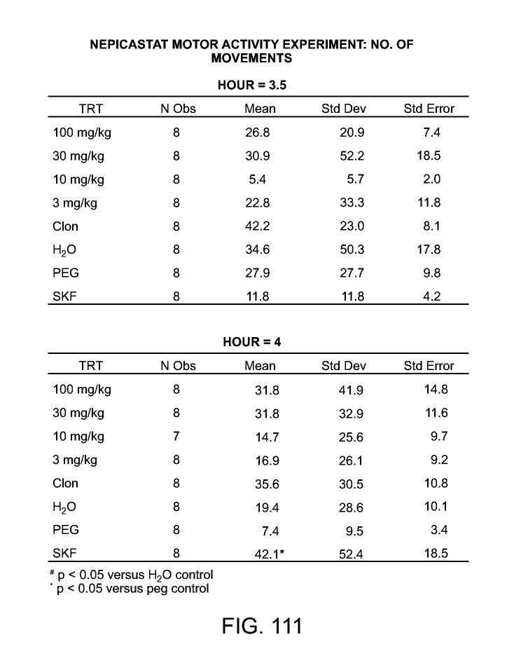

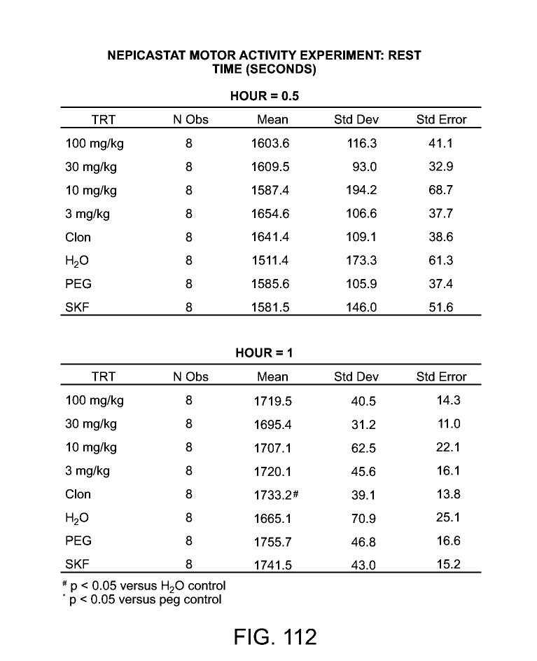

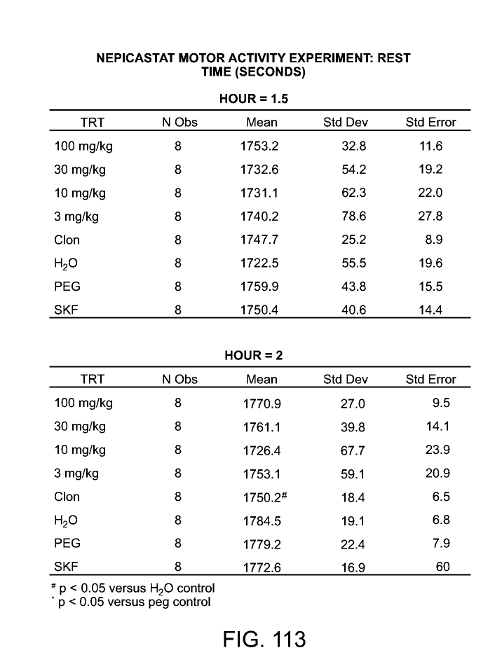

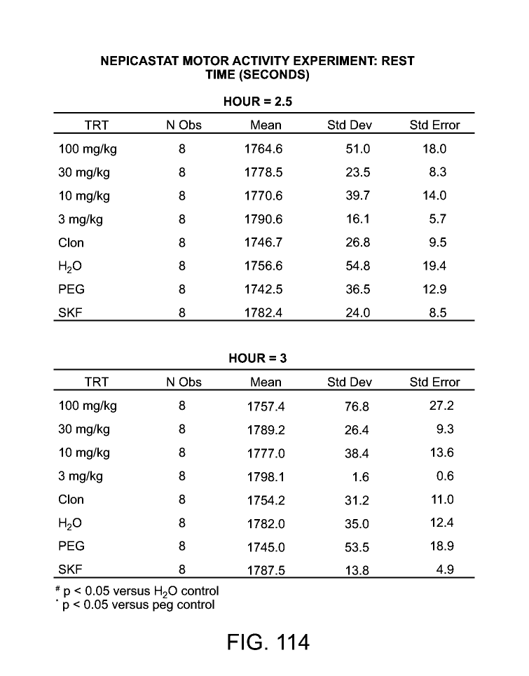

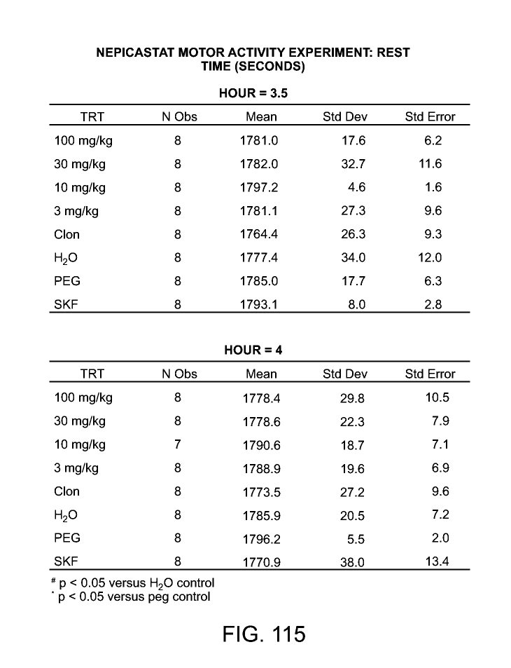

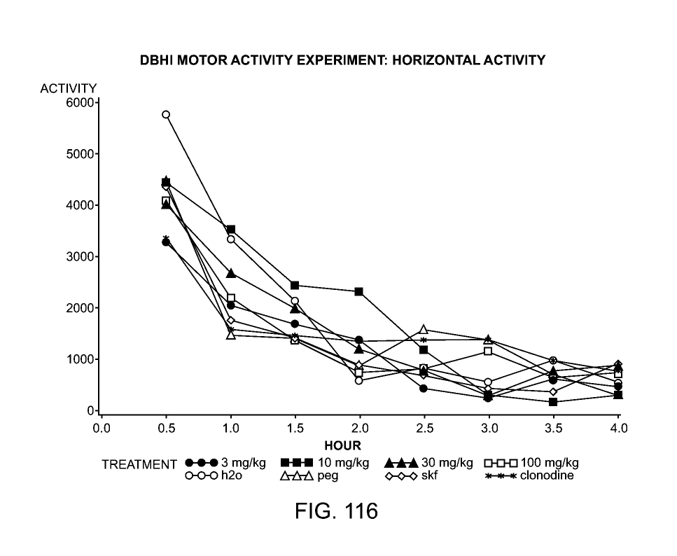

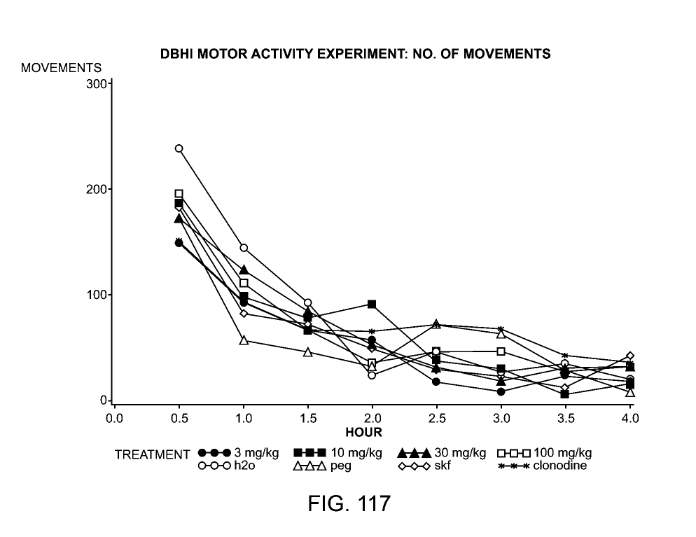

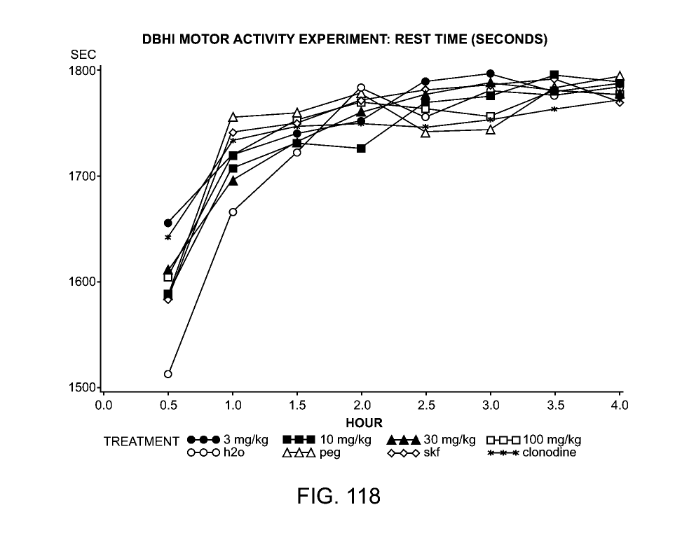

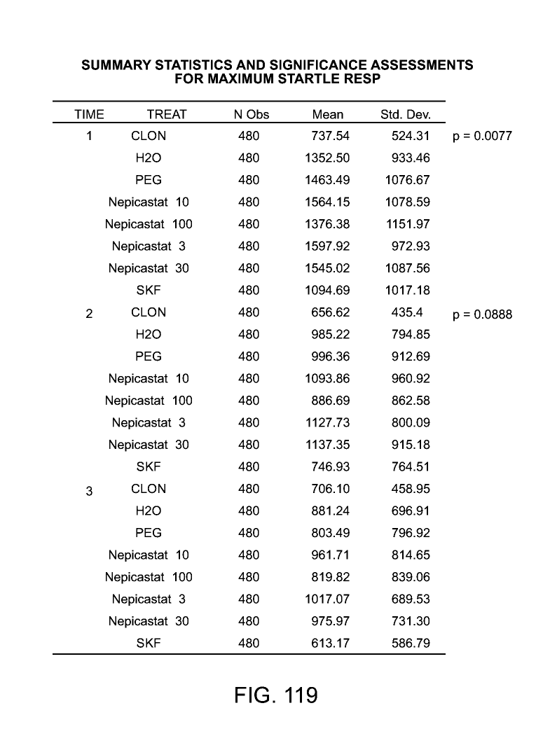

The results of analyses for the 3 parameters (horizontal activity, no. of movements and rest time) are presented in FIGS. 104-115. The plots of each parameter versus hours by treatment group are displayed in FIGS. 116-118.

When the four nepicastat-treated groups were compared to the vehicle-treated controls, there were no overall no pairwise significant differences at any time examined in any of the 3 parameters.

When compared to the vehicle-treated controls, the clonidine-treated group had significantly more horizontal activities at 2 and 2.5 hours, significantly more movements at 2 hours, and significantly less rest time at 2 hours (all p<0.05, see FIGS. 104-115 respectively). Note that the clonidine-treated group had significantly more rest time than the vehicle-treated controls at 1 hour (p<0.05).

When compared to the vehicle-treated controls, the SKF-102698-treated group had significantly less horizontal activities and significantly less movements at 2.5 hours (both p<0.05, see FIGS. 104-115. Note that the SKF-102698-treated group had significantly more movements than the vehicle-treated controls at 1.5 and 4 hours (both p<0.05). No significant differences between SKF-102698 and vehicle were detected at any time examined in the rest time (see FIGS. 104-115)

In general, the horizontal activity and number of movements decreased for the first 2 hours and stayed low for the last 2 hours. Similarly, the rest time increased for the first 2 hours and remained elevated for the last 2 hours.

nepicastat had no significant effects on the locomotor activity in rats. Animals treated with 3, 10, 30 or 100 mg/kg of nepicastat were not significantly different from the vehicle-treated controls at any time examined in the horizontal activity, no. of movements or rest time.

Animals treated with the alpha₂-adrenoceptor agonist clonidine had significantly more rest time than the vehicle-treated controls at 1 hour. However at 2 hours, animals treated with clonidine had significantly more horizontal activities and movements, and significantly less rest time, as compared to the vehicle-treated controls.

Animals treated with SKF-102698 had significantly less horizontal activities and movements than the vehicle-treated controls at 2.5 hours. The rats treated with SKF-102698 had significantly more movements at 1.5 and 4 hours, as compared to the vehicle-treated controls. No significant differences between SKF-102698 and controls were detected at any time examined in the rest time.

In startle response, the overall treatment effects for nepicastat and vehicle were not significant (p>0.05) at any time for either response. The overall treatment effect for average startle response at time 2 was marginally significant (p=0.0703), and Dunnett's test revealed that nepicastat 30 mg/kg had a significantly higher average startle response than the vehicle group (p<0.05). Baseline average startle response was statistically significant at times 3 and 4 for both responses (p≦0.05), and marginally significant at times 1 and 2 for maximum startle response, and at time 2 for average startle response (p≦0.010).

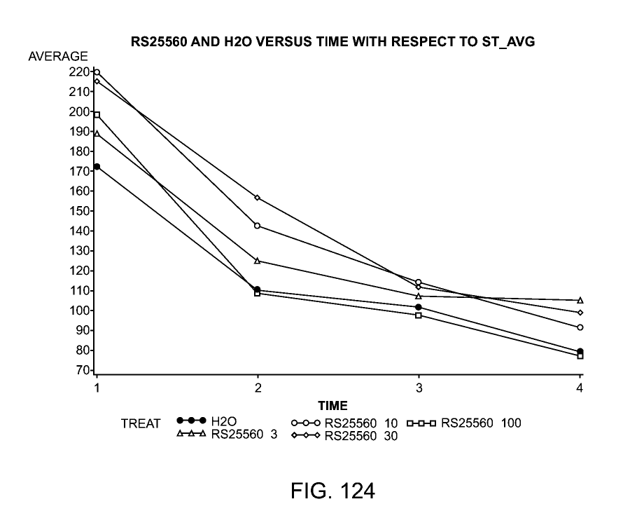

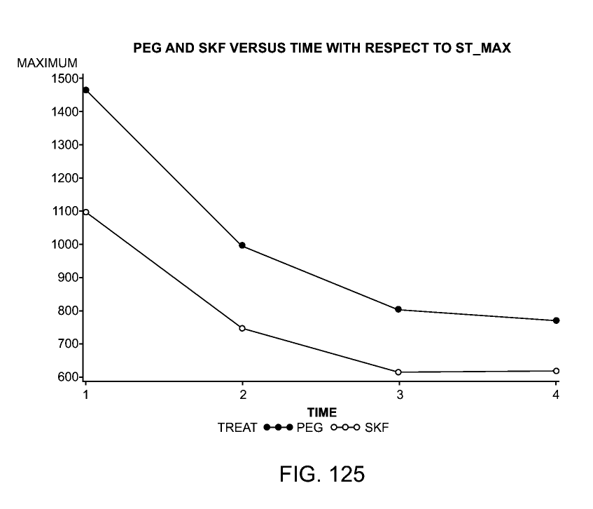

FIGS. 123-124 show the mean maximum and average startle responses versus time for each of these five treatment groups.

SKF-102698 (100 mg/kg) was not statistically significantly different from vehicle at any time for either startle response measurement.

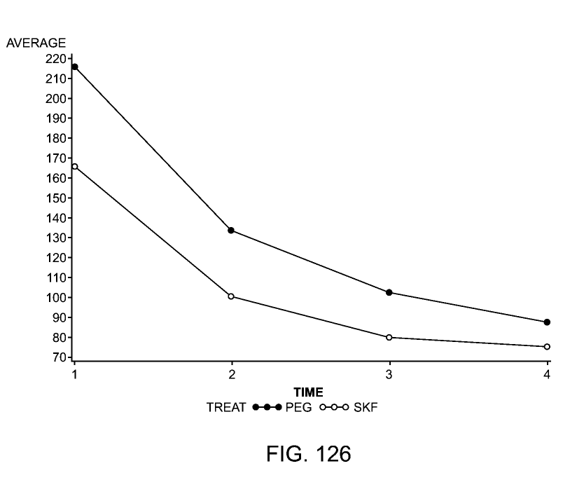

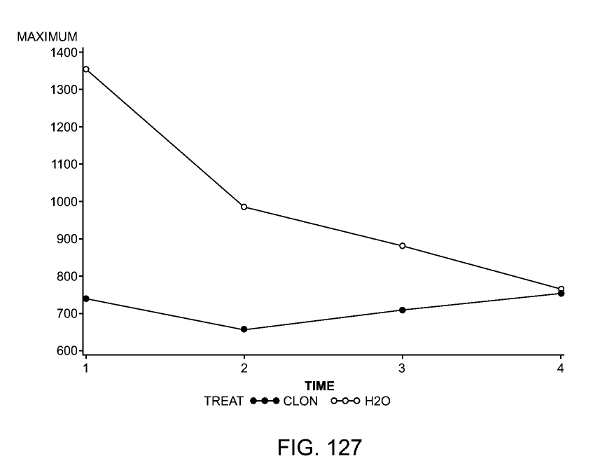

FIGS. 125 and 126 show the time course for mean maximum and average startle responses for SKF-102698 and vehicle.

Clonidine had statistically significantly lower maximum and average startle responses than vehicle at time 1 (p<0.01) and at time 2 for average startle only (p=0.0352). The maximum startle response at time 2 and the average startle response at time 3 for the clonidine group were marginally significantly lower than the water group.

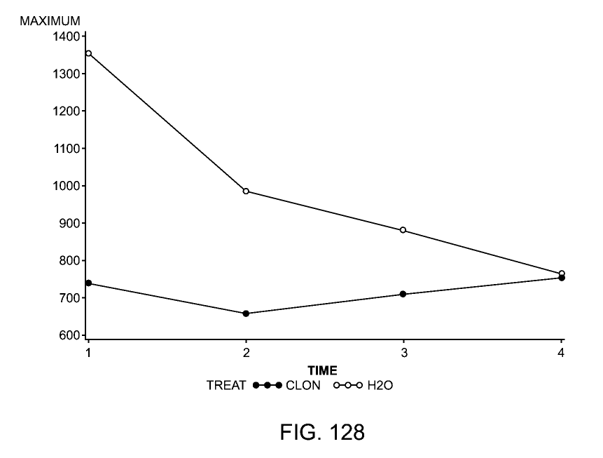

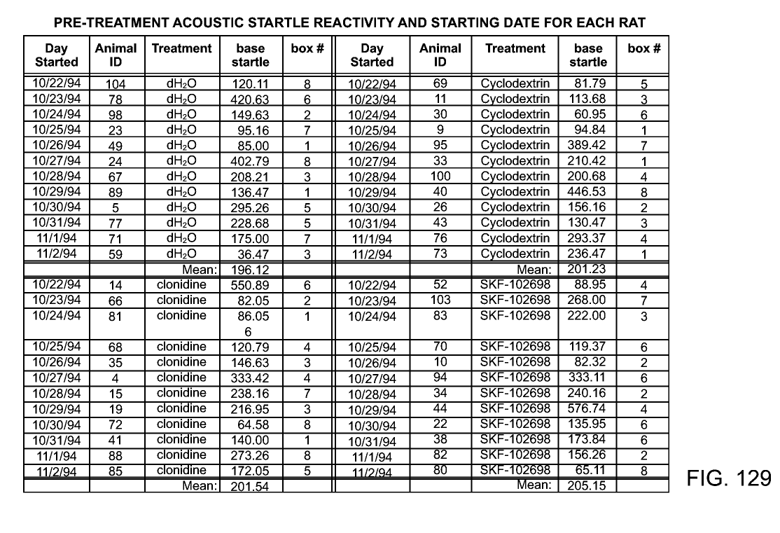

FIGS. 127-128 show the time course for mean maximum and average startle responses for clonidine and water.

nepicastat administered at 3, 10, 30, or 100 mg/kg does not appear to effect the maximum or average startle response in rats at any time when compared to vehicle. SKF-102698 behaved similarly to vehicle (PEG) for both startle responses at all times. Clonidine successfully lowered both maximum and average startle response during earlier times, and behaved similarly to vehicle during later times.

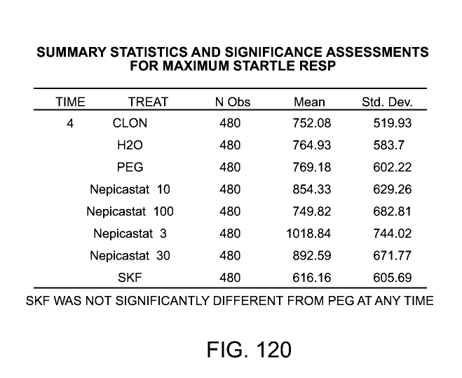

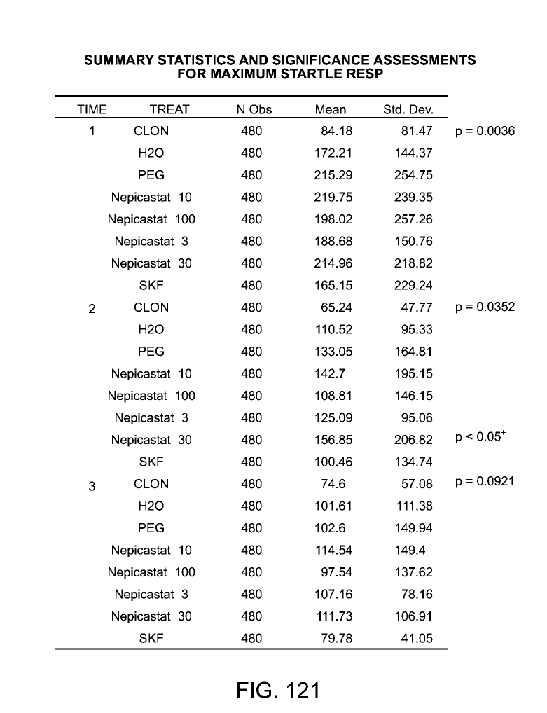

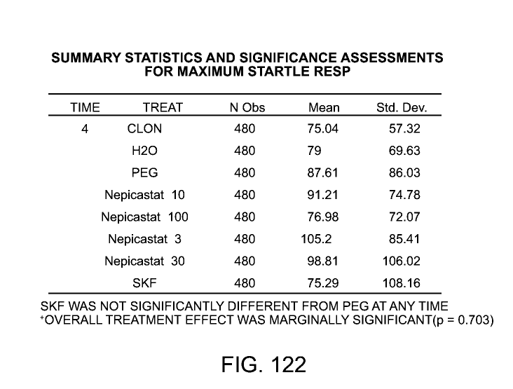

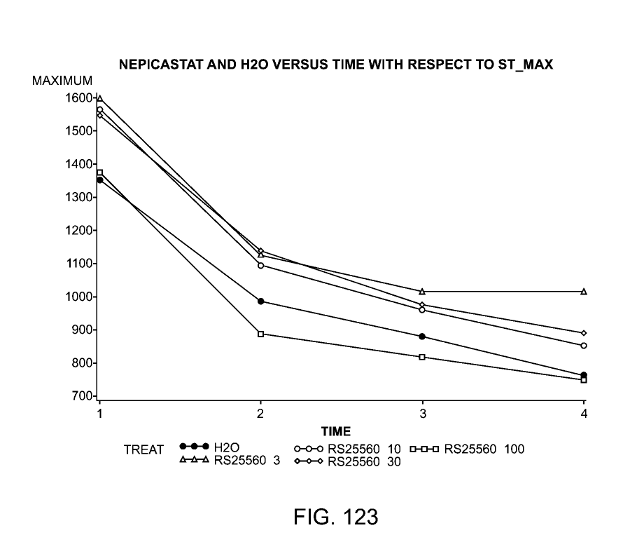

FIGS. 119-122 show summary statistics and significance assessments for maximum startle response.

### Example 22

The effects of chronic dosing of nepicastat were examined. Between three and thirteen days prior to the first dosing day the rats were placed inside the startle apparatus and after a five minute acclimation period they were presented with a 118 dB noise burst on average once a minute (a variable inter-trial interval ranging between 30 and 90 seconds will be used) for 20 minutes. The startle responses were measured and a mean for the last twenty startle response was calculated for each rat. The rats were randomly placed in one of the eight treatment groups (nepicastat, 5, 15 or 50 mg/kg, bid; SKF-102698, 50 mg/kg, bid; clonidine, 20 ug/kg, bid: d-amphetamine, 2 mg/kg, bid; dH₂O or cyclodextrin (SKF-102698's vehicle). Rats were dosed by oral gavage with a 10 ml/kg dosing volume. The rats were dosed in the morning and in the evening every day for ten day. The time in between morning and evening dosing will be between 6 and 10 hours. Previous work has shown that this matching procedure to be the most appropriate for acoustic startle reactivity since there is significant variability in startle response between rats, but a high degree of consistency within rats from one day to the next.

Since it was impossible to test all 96 rats (8 treatment groups, n=12) on the same day, the dosing schedule was staggered such that only 8 rats were run every day. These 12 groups of eight rats each consisted of one rat from each of the eight treatment groups so that the treatment groups were balanced across days. Furthermore, all treatment groups were balanced across the eight motor activity chambers, however, treatment groups could not be balanced across the startle chambers.

The following behavioral tests were administered during and after chronic dosing; body core temperature, motor activity, acoustic startle reactivity, and pre-pulse inhibition of acoustic startle.

The animals were tested in an enclosed room with a white noise generator running. Motor activity tests were conducted immediately after the body core temperature reading taken on dosing day ten (about 3 hours and 35 minutes after the morning daily dose of nepicastat, and SKF-102698, and 20 minutes prior to the daily administration of clonidine and d-amphetamine on dosing day ten). Motor activity tests were run for one hour. A diagnostic program was run on each of the motor activity chambers prior to each test session to assure that the photo beams and light sensors were operating properly. Motor activity has been shown to be sensitive to changes in central dopamine levels (Dietze and Kuschinsky, 1994) which makes this behavioral test a potential sensitive assay to the effects of DBHI in-vivo. D-amphetamine was used as the positive control for this assay.

Rat body core temperatures were obtained by inserting the rectal probe 2 cm into the colon of each rat. Each rat's body core temperature was measured three times and the average of the three reading was calculated. Body core temperature readings were obtained immediately prior to the ten day chronic dosing schedule (to obtain a baseline), and three and half hour after the morning daily dose of nepicastat, and SKF-102698, and 15 minutes prior to the daily administration of clonidine and d-amphetamine, on dosing days one, five and ten. Body core temperature has been shown to be sensitive to both dopamine and norepinephrine levels, which makes this behavioral test a potential sensitive assay to the effects of DBHI in-vivo. Both clonidine (an alpha₂ agonist), and d-amphetamine (a dopamine releaser) were used as the positive controls for this assay.

Acoustic startle reactivity (a series of muscle contractions elicited by an intense burst of noise with a rapid onset), and pre-pulse inhibition (sensorimotor gating measured by analyzing any decrease in startle reactivity which occurs when a startling stimulus is immediately preceded by a non startling stimulus) were both measured in eight SR-Lab (San Diego Instruments, San Diego, Calif.) test stations. The rats were placed individually in a Plexiglas cylinder (10 cm diameter) which was housed in a ventilated sound-attenuating enclosure. Acoustic noise bursts (a broad band noise with a rise time and fall time of 1 msec) were presented via a speaker mounted 30 cm above the animal. Also, these speakers produced a 68 dB level of background noise throughout all test sessions. A piezoelectric accelerometer attached below the plexiglas cylinder transduced the subject's movement into a voltage which was then rectified and digitized (on a scale from 0 to 4095) by a PC computer equipped with SR-Lab software and interface assembly. A decibel meter was used to calibrate the speakers in each of the eight test station to ±1% of the mean. Additionally, a SR-Lab calibrating instrument was used to calibrate each of the eight startle detection apparatuses to ±2% of the mean. Startle reactivity and pre-pulse inhibition tests were run concurrently immediately alter the motor activity test (about 4 hours and 40 minutes after the morning daily injection of nepicastat, and SKF-102698, and 10 minutes after a supplemental administration of clonidine and d-amphetamine on dosing day ten). The startle reactivity and pre-pulse inhibition tests consisted of placing each rat individually into a SR-Lab test station and after a five minute acclimation period the rats were presented with one of three different types of noise bursts (and startle reaction measured) on average once a minute (a variable inter-trial interval ranging between 30 and 90 seconds was used) for an hour (60 total noise bursts and startle reactions). The three different types of noise bursts consisted of a loud noise burst (118 dB), and a relatively quite noise burst (77 dB), the quite burst preceding the loud noise bursts by 100 msec (pre-pulse inhibition trial). These trials were presented in pseudo-random order. Pre-pulse inhibition has been shown to be sensitive to changes in mesolimbic dopamine levels. Furthermore, acoustic startle reactivity has also been shown to be sensitive to changes in dopamine and norepinephrine levels which makes these behavioral test a potential sensitive assay to the effects of DBHI in vivo. Clonidine and d-amphetamine served as the positive control for the acoustic startle reactivity and pre-pulse inhibition of acoustic startle tests.

The schedule of daily behavioral tests was as follows. At t=0, DBHI is injected. At 3.5 hours, the core body temperature is measured. At 3 hr. 35 minutes, motor activity is assessed. At 4 hr. 40 minutes, startle reactivity and pre-pulse inhibition are assessed.

Three temperature readings were taken from each subject per time of testing. The avenge of these three readings was then calculated.

Each rats spontaneous locomotion was obtained by calculating the total number of photobeams that the subject broke during the testing session.

The subject's reaction was measured during each trial for the 40 msec window after the stimulus was presented. Each startle reaction was calculated by taking the avenge of 40 readings (one per millisecond) starting immediately after each noise burst. Acoustic startle reactivity was calculated by determining the mean response for each subjects startle elicited by the 118 dB acoustic burst. Pre-pulse inhibition values were calculated by subtracting the mean startle response elicited by the 77 dB pulse-118 dB pulse paired trial (pre-pulse inhibition trial described above) from the 118 dB alone trial and then dividing this value by the 118 db alone trial for each rat, i.e. ([118 dB trial value−pre-pulse inhibition trial value] 118 db trial value).

An overall one-way ANOVA with a main effect for treatment was performed at each time on the change from baseline for each animal. Subsequent t-tests were performed for each comparison of interest.

Spontaneous motor activity was measured for each animal every 15 min for 1 hour. Each time block (every 15 min) was analyzed separately. Kruskal-Wallis test (nonparametric technique) was performed to test for the difference between treatment groups. If the overall significant difference is not detected, Bonferroni's adjustment for multiple comparisons is then made.

The mean average voltage (AVGMEAN) and mean percent prepulse inhibition (RATIO) were computed for each treatment (TREAT) and trial type (TRIALT) at each trial (TRIALN). Pre-pulse inhibition values were calculated by subtracting the mean startle response elicited by the 77 dB pulse−118 dB pulse paired trial (pre-pulse inhibition trial described above) from the 118 dB alone trial and then dividing this value by the 118 db alone trial for each rat, i.e. ([118 dB trial value−pre-pulse inhibition trial value]÷118 db trial value).

These values were plotted against trial number for each treatment and trial type, and these plots are attached to the report. Note that the y-axis on the plots varies. The trials 1-15 correspond to time 1, 16-30 time 2, 31-45 time 3, and 46-60 time 4. Plots displaying the mean percent prepulse inhibition and the mean average startle of animals over TIME for each treatment are attached also.

The average startle response and the percent prepulse inhibition were analyzed using Analysis of Variance. The model included terms for treatment, animals nested within treatment, time and treatment by time interaction. Treatment effects were tested using the error term for animals nested within treatment. Overall treatment effects and treatment effects by time were studied. The method of Fisher's Least Significant Differences was used to adjust for multiple comparisons. If the overall treatment or treatment by time effects were not significant (p-value>0.05) then a Bonferroni adjustment was made. If the overall treatment effects were nonsignificant, then the adjustment was applied to the specific pairwise comparisons. Further, if the specific pairwise treatment effect was not significant (p-value >0.05), then the adjustment was also applied to the treatment effects within time. If both the overall treatment and treatment by time effects were not significant (p-value >0.05) then a Bonferroni adjustment was made for the individual comparisons within time and averaging over time.

The change from pre-dose in body weights was calculated for each animal for the analysis. A repeated measures two-way ANOVA was used to test for the overall effects of treatment, time and treatment by time interaction. One-way ANOVAS were then performed to test the treatment effect at each day.

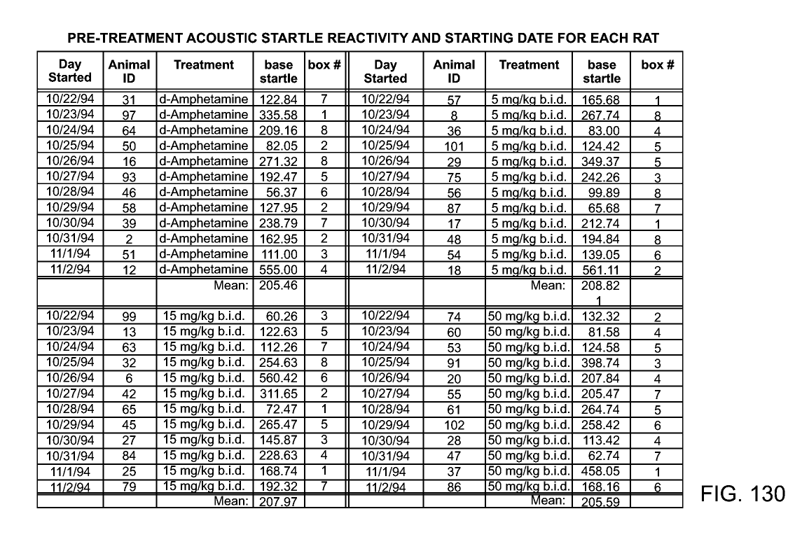

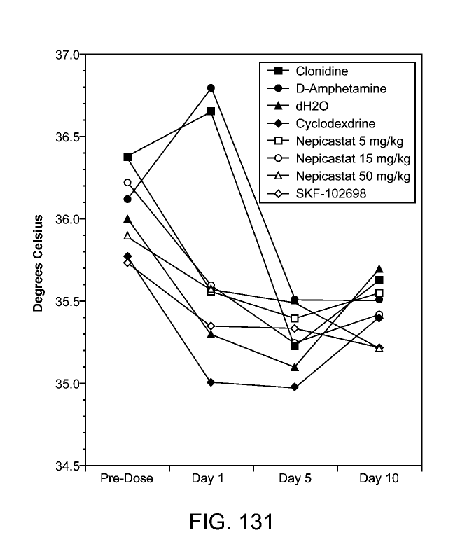

FIG. 129-130 show pre-treatment acoustic startle reactivity and starting date for each rat.

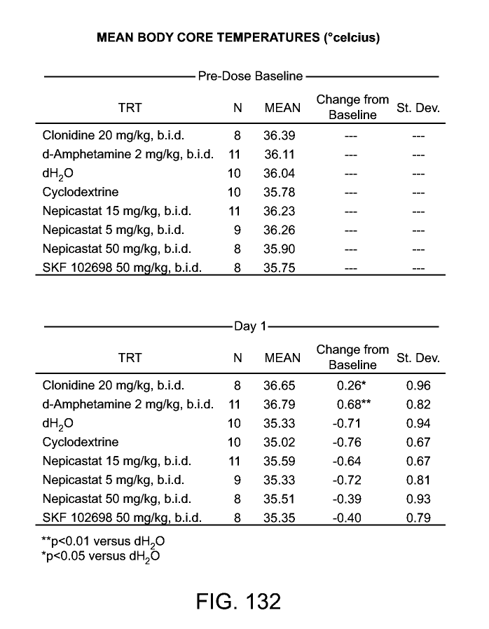

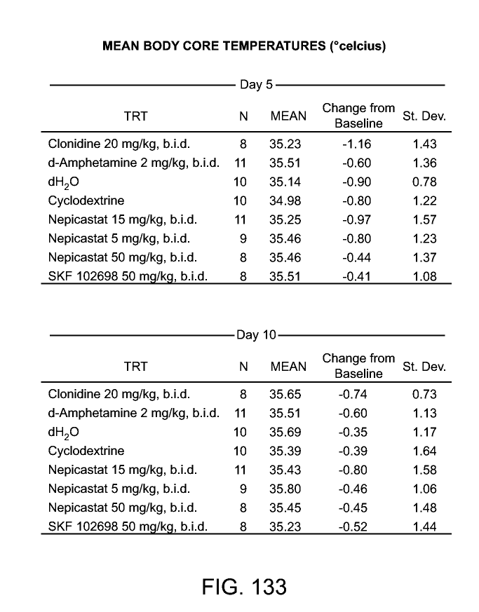

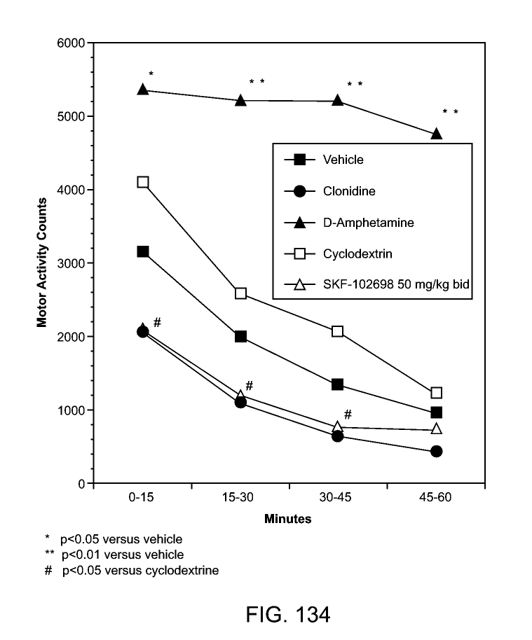

FIG. 131 shows that other than the positive controls (d-amphetamine and clonidine) significantly increasing body core temperature on day one of the chronic dosing, no other compound had any significant effect on body core temperature at any time. FIGS. 132-133 contain the mean body core temperature at each time for each treatment, the mean change in core body temperature from baseline, and significance results.

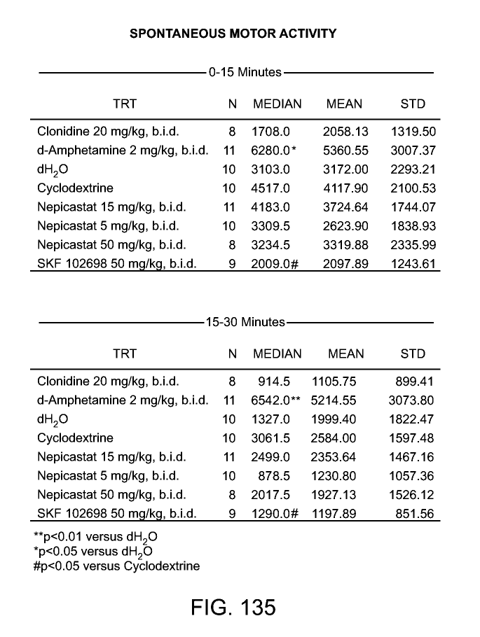

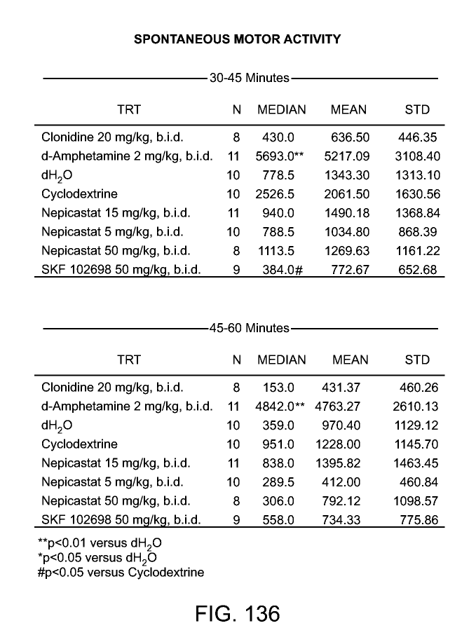

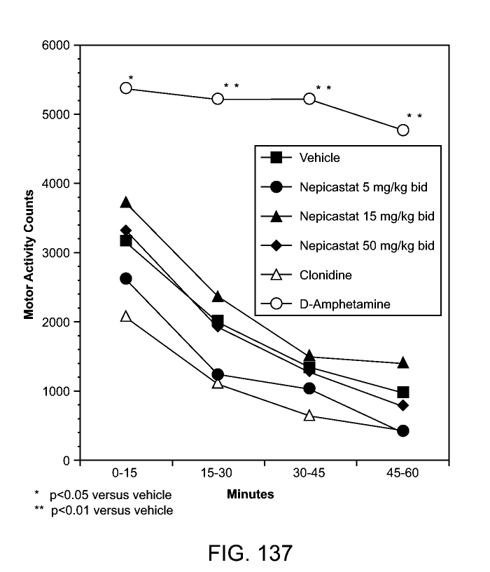

As FIG. 134 shows, the d-amphetamine group had significantly higher locomotor activity than the vehicle control at all times examined. The clonidine group, however, was not significantly different from the vehicle controls at any time examined. The SKF 102698 50 mg/kg b.i.d. group had significantly lower locomotor activity than its vehicle control at the first 45 minutes (i.e. samples 1-3), but not significant after 45 minutes (see FIGS. 135-136).

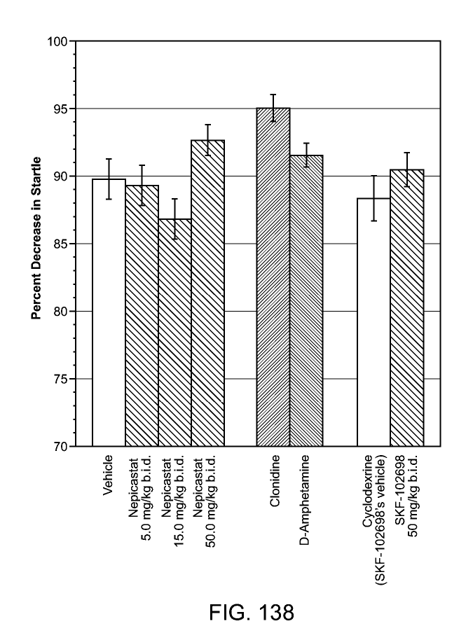

FIG. 137 also shows that there was no overall significant treatment effect for nepicastat at any time examined. Pairwise comparisons revealed that none of the nepicastat-treated groups were significantly different from the vehicle controls at any time examined. Also, there was no significant difference between the two vehicle controls ((dH₂O and SKF's vehicle) at any time examined (see FIG. 135 and FIG. 136).

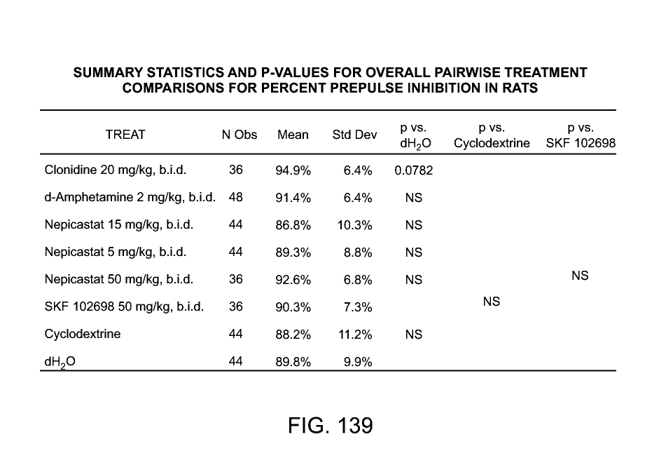

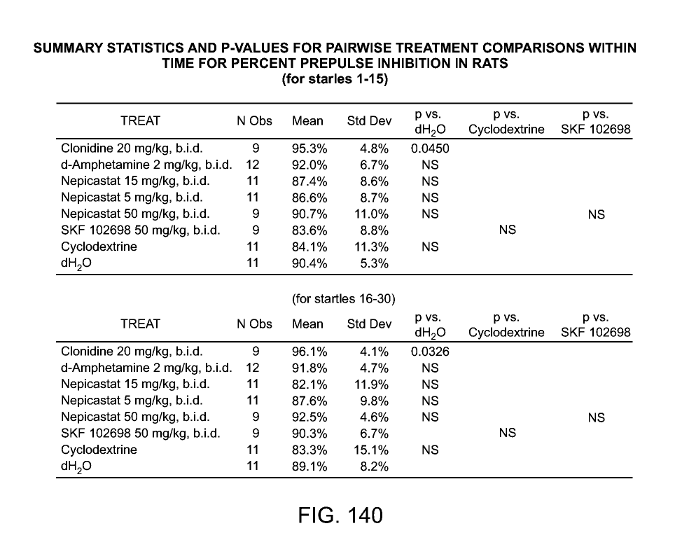

As FIG. 138 shows, none of the treatment groups produced any significant change in pre-pulse inhibition. The overall time effect was statistically significant for the SKF 102698 group and the cyclodextrin group (p=0.0001). The treatment by time interaction was statistically significant for cyclodextrin versus dH₂O (p=0.0283), but no others. Treatment effects were not significant for any comparisons of interest. However, the SKF group had marginally higher percent prepulse inhibition compared to the cyclodextrin group (p=0.0782) (see FIG. 139).

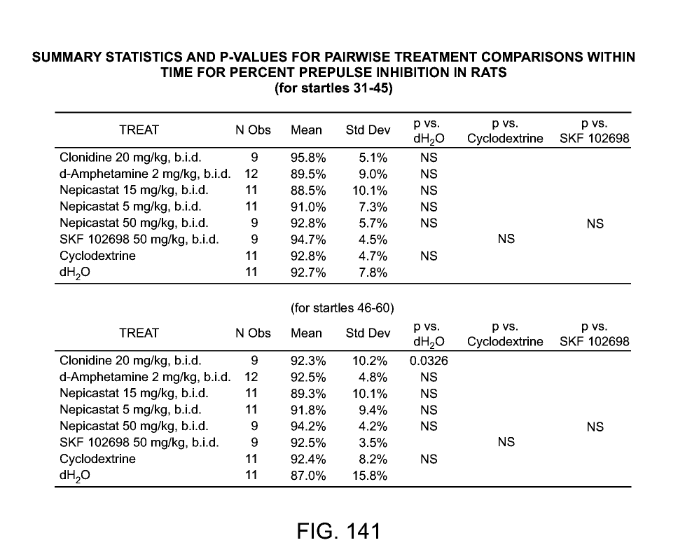

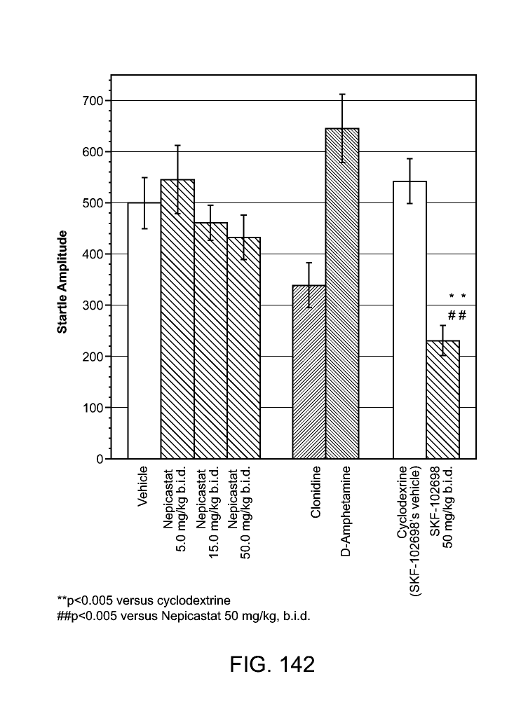

During times 1 and 2, the clonidine group had just significantly higher percent prepulse inhibition than the vehicle control and were not significantly different from vehicle during times 3 and 4 (see FIG. 140-141). Neither d-amphetamine nor SKF 102698 was significantly different from their own vehicle at any time. None of the nepicastat dose groups were significantly different from dH₂O at any time.

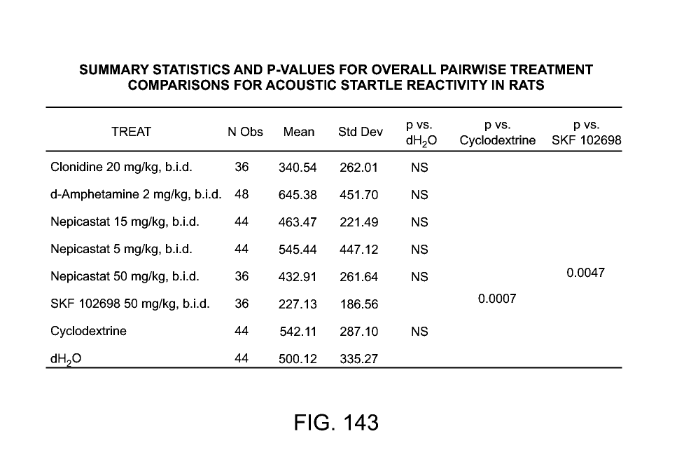

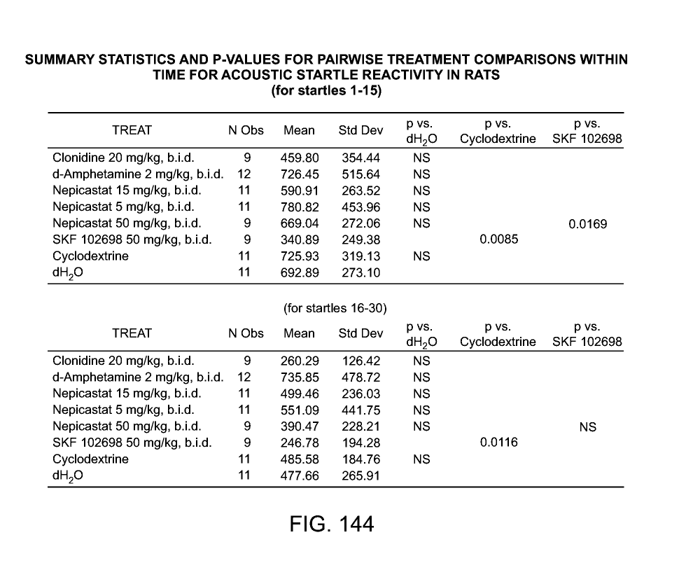

As FIG. 142 shows, only the SKF 102698 treatment group produced a significant change in acoustic startle reactivity. The overall time effect was statistically significant for all comparisons of interest (all p=0.0001). The treatment by time interaction was statistically significant for the comparisons of amphetamine versus dH₂O, clonidine versus dH₂O and cyclodextrin versus dH₂O (all p<0.05), but no others. Treatment effects were significant for SKF 102698 50 mg/kg b.i.d. versus cyclodextrin (p=0.0007) and for nepicastat 50 mg/kg b.i.d. versus SKF 102698 50 mg/kg b.i.d. (p=0.0047), but no others (see FIG. 143). The SKF 102698 50 mg/kg b.i.d. group had significantly lower startle response compared to cyclodextrin, and also had significantly lower startle response as compared to the nepicastat 50 mg/kg b.i.d. group.

The SKF 102698 (50 mg/kg b.i.d.) group had significantly lower startle response than the cyclodextrin group at all times (see Table 144-145). During times 1 and 3, the nepicastat (50 mg/kg b.i.d.) group had significantly higher startle response than the SKF 102698(50 mg/kg b.i.d.) group. No other significant differences were detected.

There was no overall or pairwise significant differences in body weight between groups at the pre-dose baseline.

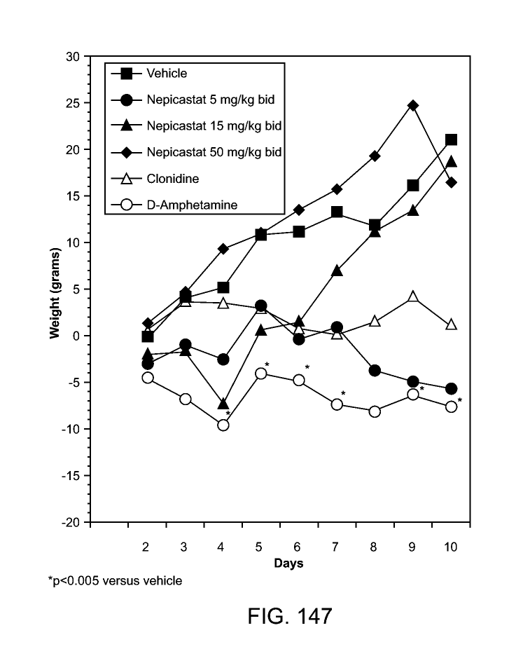

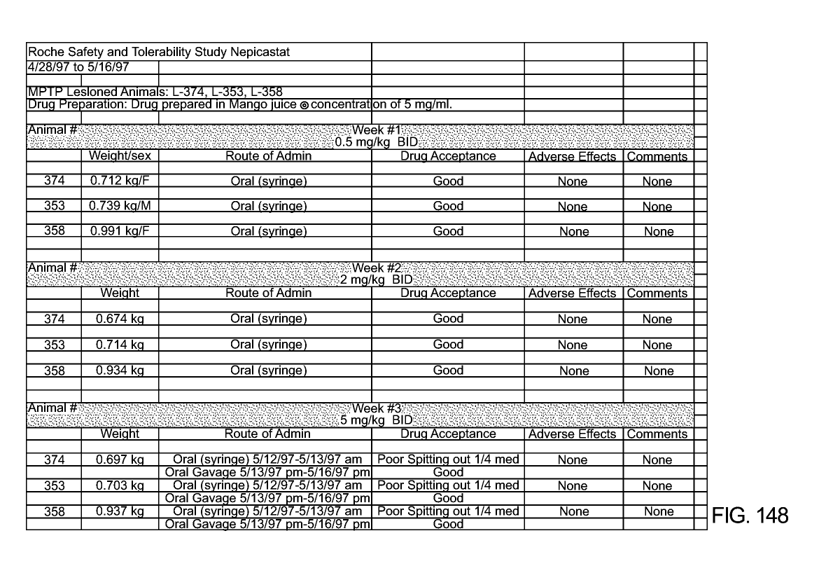

As shown in FIGS. 146-147, the d-amphetamine group had a significantly smaller change in body weight from pre-dose than the vehicle controls (p<0.01). When analyzed within each day, the vehicle controls had a significantly greater increase from pre-dose in body weight than the amphetamine group at treatment days 4-10. The clonidine group, however, was not significantly different from the vehicle controls at any time examined. As illustrated in FIG. 146, the SKF 102698 (50 mg/kg b.i.d.) group showed a significantly smaller increase (p<0.01) in body weight from pre-dose baseline than its vehicle control (SKF-vehicle). When analyzed within each day, the SKF-vehicle controls had a significantly greater increase from pre-dose in body weight than the SKF 102698 group at treatment days 2-10, except days 3 and 6. Importantly, there was no difference in changes in body weight between the SKF-vehicle and the vehicle control groups on any day.

As shown in FIG. 147, there was no overall significant treatment effect for any dose of nepicastat at any time examined. Pairwise comparisons revealed that none of the nepicastat-treated groups were significantly different from the vehicle controls at any time examined. Interestingly, there was a significant (p<0.05) overall difference between the SKF 102698 (50 mg/kg b.i.d.) group and the nepicastat (50 mg/kg b.i.d.) group with respect to changes in body weight. When analyzed within each day, the SKF 102698 (50 mg/kg b.i.d.) group had significantly lower body weights than the nepicastat (50 mg/kg b.i.d.) group at days 7-9.

### Example 23

1-methyl-4-phenyl-1,2,3,6-tetrahydropyridine (MPTP) was purchased from RBI, Inc, (Natick, Mass.). For administration, MPTP was suspended in water at a concentration of 2 mg/ml (free base) and was injected subcutaneously in a volume (ml) equal to the weight (kg) of each animal. For example, a 950 gram animal received an injection of 0.95 ml of MPTP at 2 mg/mI resulting in 2.0 mg/kg final per injection.

Twenty-seven (27) mature squirrel monkeys (Saimiri sciureus), sixteen females and eleven males were used for this study. The monkeys were maintained on a 13 h/11 h light-dark cycle, with food and water available ad libitum. All procedures used in this study followed NIH guidelines and were approved by the Institutional Animal Care and Use Committee (IACUC). Animals were individually housed and allowed a minimum of one month to acclimate to the colony prior to commencing behavioral studies. A total of six squirrel monkeys, three non-lesioned and three lesioned (received 2 mg/kg MPTP 3 months prior), were used for these studies. To determine the optimal route of administration of drug nepicastat, three different approaches were examined including (i) insertion into treats, (ii) oral syringe, and (ii) oral gavage. (i) Insertion of RS solution (5 mg/ml) into marshmallows was tested in 3 non-lesioned monkeys and proved to be a poor route of drug administration due to failure of animals to ingest treats probably due to adverse taste. (ii) Oral syringe injection of drug (0.5, 2, and 5 mg/kg) into the mouth of three non-lesioned and three lesioned monkeys was also not an acceptable route since animals tended to spit out the solution at the highest drug concentration. (iii) Oral gavage administration was carried out in 3 MPTP-Iesioned monkeys at the highest dose (5 mg/kg) and was well accepted.

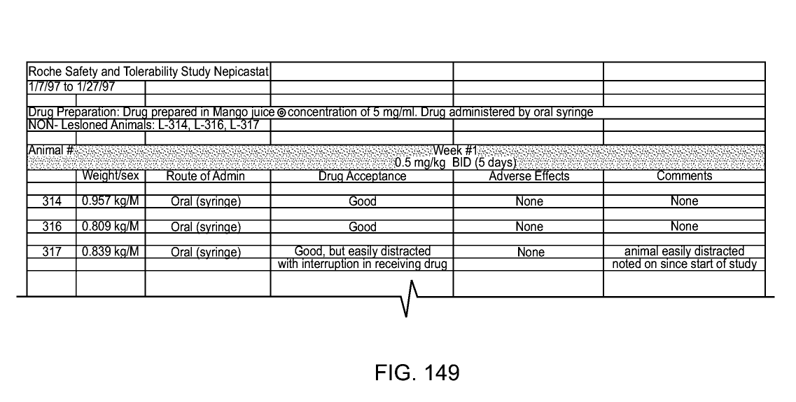

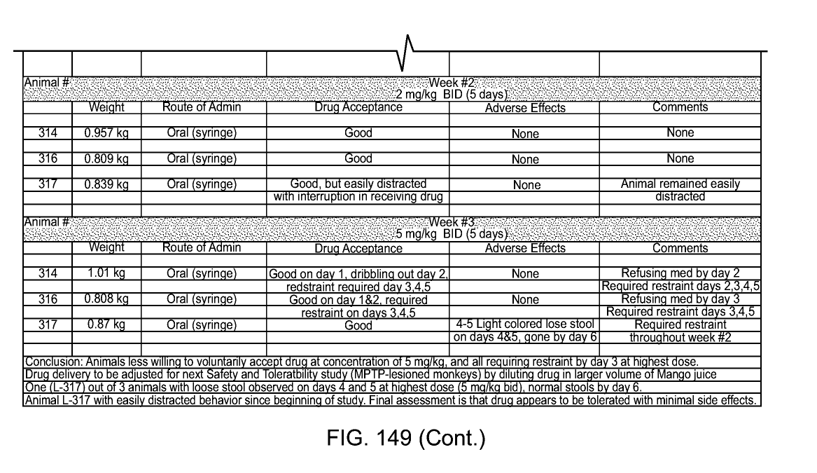

These results (oral syringe and oral gavage delivery) are summarized in FIG. 148 and FIG. 149.

A total of six squirrel monkeys, three non-lesioned and three lesioned (received 2 mg/kg MPTP 3 months prior), were used for these studies. Animals received drug at a concentration of 0.5, 2.0, or 5.0 mg/kg twice daily, (10 am and 2 pm), for 5 days with a two-day washout between the different dose levels. Drug was administered via oral syringe at the 0.5, 2.0 and 5.0 mg/kg doses and as oral gavage at the 5.0 mg/kg dose. Drug was well tolerated at the two lower doses. One non-lesioned monkey receiving 5.0 mg/kg had light beige colored loose stools on the final two days of administration that resolved upon one day withdrawal of drug. These results are summarized in FIGS. 148 and 149.

A total of 24 squirrel monkeys (Saimiri sciureus), fourteen females and ten males were used in this study. The twenty-four animals were randomly assigned to one of four treatment groups, with 6 animals per group. The groups consisted of the following (1) group A: placebo (2) group B: 1 mg/kg/day; (3) group C: 4 mg/kg/day; and (4) group D: 10 mg/kg/day. In Group B, one animal died acutely following MPTP-lesioning, and was not replaced.

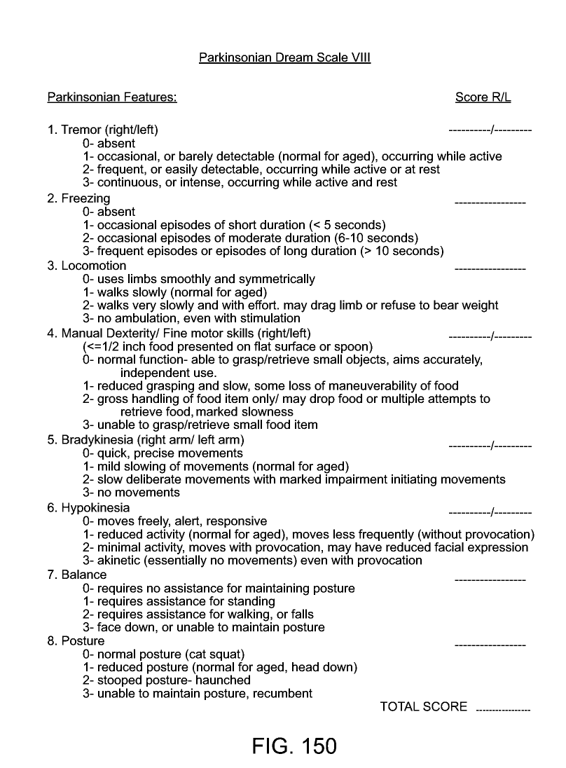

Prior to lesioning, animals were subjected to quantitative assessment of spontaneous motor activity using an infrared activity monitor (IRAM) cage. All recording sessions were 60 minutes in length and were carried out for 10 sessions over a period of 2 weeks. The behavior of animals was also assessed by 1 to 3 clinical raters using a parkinsonian clinical rating scale (CRS) once per day (12 noon to 1 pm) for 3 to 5 consecutive days. Normal animals did not typically score greater than 3 on the CRS (see FIG. 150 for the clinical rating scale used in these studies). Both the activity monitoring (IRAM) and clinical rating assessments established the mean base-line activity of each animal.

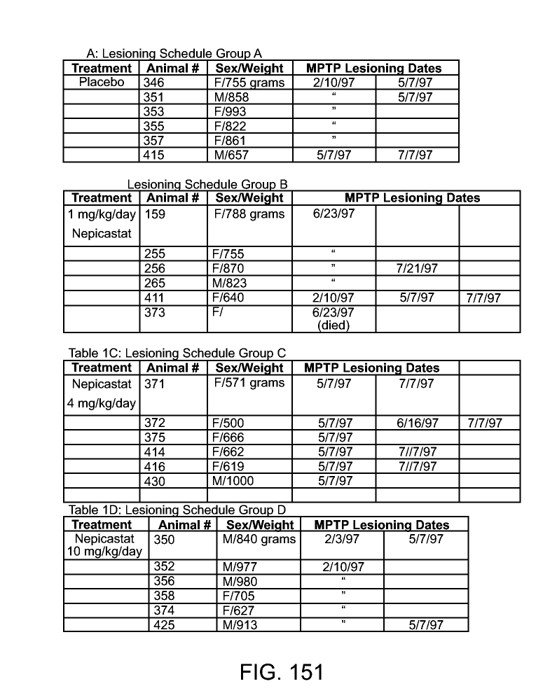

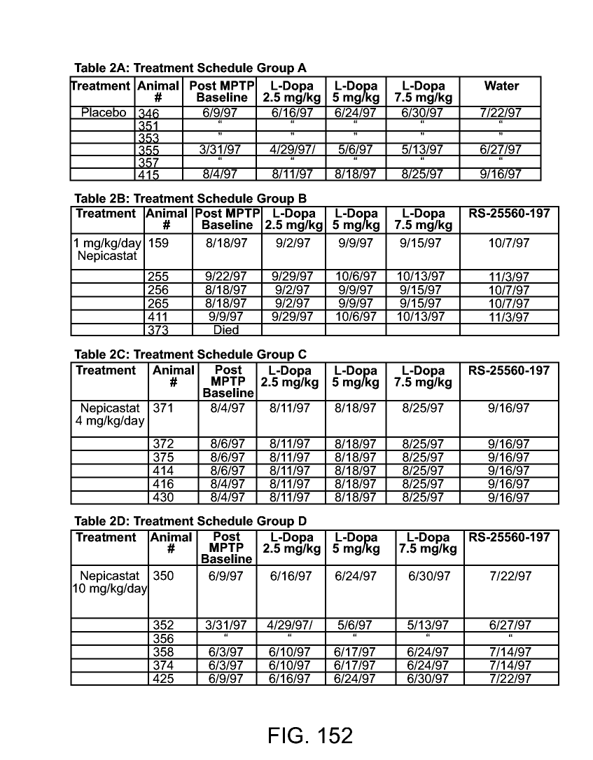

Animals were lesioned by the administration of MPTP at a concentration of 2.0 mg/kg (free-base) via subcutaneous injection to achieve a parkinsonian state (see FIGS. 151-156). A post-MPTP lesioning behavioral assessment was carried out 2 to 4 weeks after the last MPTP-Iesioning. Locomotor activity was monitored by IRAM in 60-minute sessions for 3 to 5 days. Clinical behavior (CAS) was assessed by one to three individuals rating over a period of 3 to 5 days.

In some cases, animals required additional doses of MPTP (2 mg/kg) to obtain a sufficient degree of lesioning to display parkinsonian symptoms, defined as an average total clinical rating score greater than 3.

All animals received a final post-MPTP behavioral assessment (IRAM and CRS), within three weeks of starting the efficacy study. This final post-MPTP evaluation was used to establish a baseline clinical parkinsonian state and used as a pretreatment value for statistical analysis.

Animals were tested for response to L-Dopa and the efficacy of drug nepicastat. Testing was carried out 4 to 12 weeks after the last MPTP dose. Twenty-tour squirrel monkeys were randomly assigned to 4 groups; Group A (6 animals) received placebo (water) treatment; Group B (5 animals) received drug nepicastat at 1 mg/kg/day (0.5 mg/kg twice daily); Group C (6 animals) received 4 mg/kg/day (2 mg/kg twice daily); and Group D (6 animals) received 10 mg/kg/day (5 mg/kg twice daily).

L-Dopa was administered at a concentration of either 2.5, 5, or 7.5 mg/kg by oral gavage twice daily (at 10 am and 2 pm) for 7 consecutive days. Behavior was determined by IRAM and CRS. Clinical rating was carried out 60 to 90 minutes following the 10 am morning dose on the last 4 days of treatment. Raters (one to three individuals) were blinded to the different treatment groups. IRAM assessment were preformed for 90 minutes immediately following drug administration at 2 pm on the last 2 to 5 days of drug treatment. There was a minimum 2 day washout period between each treatment dose.

Drug nepicastat or water (as placebo) was administered for 12 days following a minimum 2 day washout after L-Dopa dosing. Drug was administered twice daily at 10 am and 2 pm by oral gavage. Behavior was rated by IRAM and CRS. The CRS was conducted in the morning, 60 to 90 minutes after the 10 am dose of nepicastat on the last 5 days of drug treatment. Raters (one to three individuals) were blinded to the different treatment groups. IRAM assessments were preformed for 90 minutes immediately following drug administration at 2 pm on the last 5 days of drug treatment.

A pharmacokinetic study was carried out to determine the plasma concentration of nepicastat in the squirrel monkey. This study was carried out concurrently with the safety and tolerability study. Three MPTP-lesioned squirrel monkeys (#353, 358 and 374) were used. One milliliter of blood (drawn from the femoral vein of each animal) was collected for analysis. Nepicastat was administered at concentrations of 1, 4, and 10 mg/kg for 5 days with a 2-day washout between each drug concentration. Blood was collected for analysis 1 hour prior to the first dose to establish baseline and at 6 hours after this first drug dose of each of the different drug levels.

A second pharmacokinetic study was carried out to determine the steady-state plasma level of nepicastat. This study was carried out concomitantly with the efficacy study where animals were tested on each of three different drug concentrations for 12 days. One milliliter of blood was drawn from the femoral vein 6 hours after the first dose on day 1, then 6 hours after the first dose on day 7, and finally 6 hours after the first dose on day 12. Baseline plasma levels were determined on samples collected the week prior to drug dosing.

The average locomotor activity was calculated pre- and post-MPTP-lesioning for each animal. The pre-MPTP-lesioning baseline was determined by averaging ten 1-hour monitoring sessions. The post-MPTP (pre-treatment) behavioral assessment was obtained within three weeks of commencing the efficacy study. The post-MPTP-lesioning locomotor activity was determined by averaging three to five 1-hour monitoring sessions (IRAMS). Activity monitoring was reported as “movements/10 minutes”. A higher score was considered a faster animal. The Wilcoxon sign rank test was used to compare pre- and post-MPTP-lesioning activity for each group of animals (groups A through D). (b) Clinical Rating Score No pre-MPTP-lesioned animal scored greater than three on the CRS. A post-MPTP clinical rating score was determined within three weeks of commencing the efficacy study by averaging the total CRS of 1 to 3 individual raters from data over 3 to 5 consecutive days.

Eight parkinsonian features were rated in each animal and the total score was derived from the sum of these eight features. For each animal, a single clinical rating score was obtained for each drug dose by averaging the clinical rating scores of all raters (one to three) conducted over the consecutive multiple dosing (with the same dose) days. This average CRS was used for statistical analysis. A lower score was considered a less parkinsonian behavioral state.

Statistical analysis consisted of:

(1) comparisons between the average CRS of placebo to nepicastat at 1, 4, and 10 mg/kg/day using the Kruskal-Wallis (non-parametric analysis of variance). This comparison was repeated using the average CRS for each experimental drug concentration corrected by the final post-MPTP ratings for each animal. The corrected clinical scores are clinical scores of experimental drug at each concentration as a ratio of post-MPTP clinical scores.

(2) Pairwlse comparisons between the average CRS post-MPTP lesioning (pre-treatment) to 2.5, 5.0, and 7.5 mg/kg L-Dopa and placebo treatment using Friedman's analysis (non-parametric analysis of variance, repeated measures). The same analysis was performed for nepicastat at concentrations of 1, 4, and 10 mg/kg. Dunnett's post hoc analysis for non-parametric data was performed when needed.

IRAM Locomotor activity was monitored every 10 minutes for a minimum of 90 minutes following each drug level. A higher rating is considered a faster (less parkinsonian) animal.

Statistical analysis consisted of descriptive statistics and graphing the mean of each 10 minutes data blocks of placebo and experimental drug at 1, 4, and 10 mg/kg. The graph was then examined to detect any trends. Further statistical analysis was not performed since no difference was determined from graphical analysis.

Statistical analysis comparing post-MPTP lesioning (pre-treatment) to 2.5, 5.0, and 7.5 mg/kg L-Dopa and nepicastat (1,4,10 mg/kg/day or placebo) was not performed due to insufficient IRAM data collection. Only 60 minutes sessions were collected at Post-MPTP, versus 90 minutes for 1 nepicastat.

Both IRAM (activity monitoring) and CRS (clinical rating scale) were used to assess the degree of MPTP-lesioning in each squirrel monkey. The following section tabulates these results for Groups A through D showing IRAM and CRS. Overall there was no significant difference in the locomotor activity as measured by IRAM between base-line (pre-MPTP-lesioning) and post-MPTP-lesioning within groups due to the high degree of variability of the RAM results for each animal. The CBS results showed a difference between pre-MPTP and post-MPTP-lesioning states. Pre-MPTP-lesioned animals scored no greater than 3 in the CRS. Post-MPTP-lesioned animals all scored greater than 3. All groups (A through D) had an average CRS ranging from 8 to 10 out of a total possible CRS score of 24.

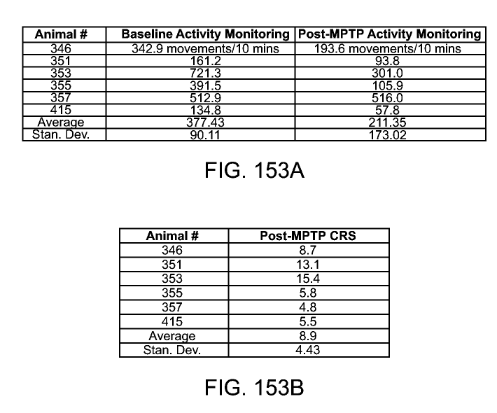

FIG. 153A shows results for Group A: Placebo Treatment. There was no significant difference between pre-lesioned and post-lesioned IRAM groups due to the high degree of variability of movements per 10 minutes per animal. Wilcoxon signed rank test: W=19, N=6, P<0.06 Accept Null Hypothesis.

FIG. 153B shows Clinical Rating Score (GRS). The average GRS for group A was 8.9, range 4.8 to 15.4. All animals showed substantial increase in the clinical rating scores after MPTP-lesioning. Normal animals (non-lesioned) typically have scores less than 3.

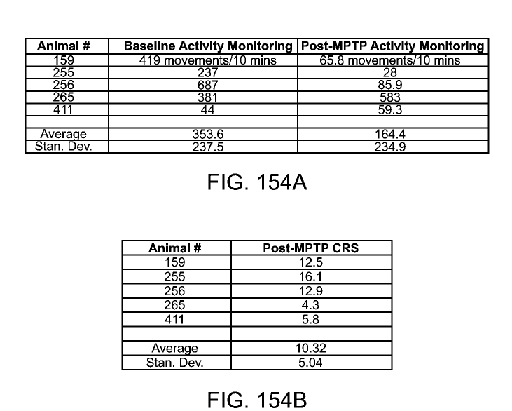

FIGS. 154 A and B show results for Group B: 1 mg/kg/day Treatment There was no significant difference between pre-lesioned and post-lesioned IRAM groups due to the high degree of variability of movements per 10 minutes per animal. Wilcoxon signed rank test: W=9, N=5, p<0.06 Accept Null Hypothesis Clinical Rating Score (CRS). The average CRS for group B was 10.32, range 4.3 to 16.1. All animals showed substantial increase in the clinical rating scores after MPTP-lesioning. Normal animals (non-lesioned) typically have scores less than 3.

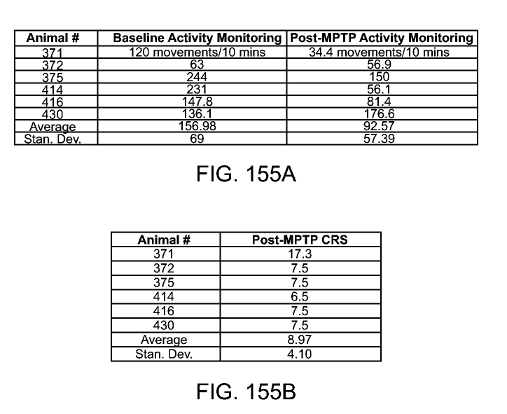

FIG. 155 shows results for Group C: 4 mg/kg/day Treatment. There was no significant difference between pre-lesioned and post-lesioned IRAM groups due to the high degree of variability of movements per 10 minutes per animal. Wilcoxon signed rank test: W=17, N=6, P>0.06 Accept Null Hypothesis The average CRS for group C was 8.97, range 6.5 to 17.3. All animals showed substantial increase in the clinical rating scores after MPTP-lesioning. Normal animals (non-lesioned) typically have scores less than 3.

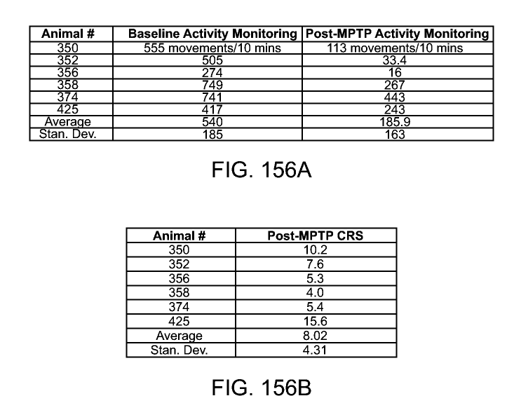

FIG. 156 shows results for Group D: 10 mg/kg/day treatment. There was no significant difference between pre-lesioned and post-lesioned IRAM groups due to the high degree of variability of movements per 10 minutes per animal. Wilcoxon signed rank test: W=21, N=6, P>0.06 Accept Null Hypothesis. All animals showed substantial increase in the clinical rating scores after MPTP-lesioning. The average CRS for group C was 8.02, range 4.0 to 15.6. Normal animals (non-lesioned) typically have scores less than 3.

There were no detectable differences between placebo treatment and three different concentrations of nepicastat (1, 4, 10 mg/kg/day) in the MPTP-lesioned squirrel monkey. Both 4 and 10 mg/kg/day of nepicastat and placebo showed a significant improvement over the post-MPTP (pre-treatment) state. All groups of animals showed significant improvement with both 5 mg/kg and 7.6 mg/kg L-Dopa when compared to post-MPTP (pre-treatment), with the exception of Group C for the 7.5 mg/kg dose and Group B for the 5 mg/kg/dose. No groups of animals demonstrated significant improvement at 2.5 mg/kg L-Dopa when compared to post-MPTP.

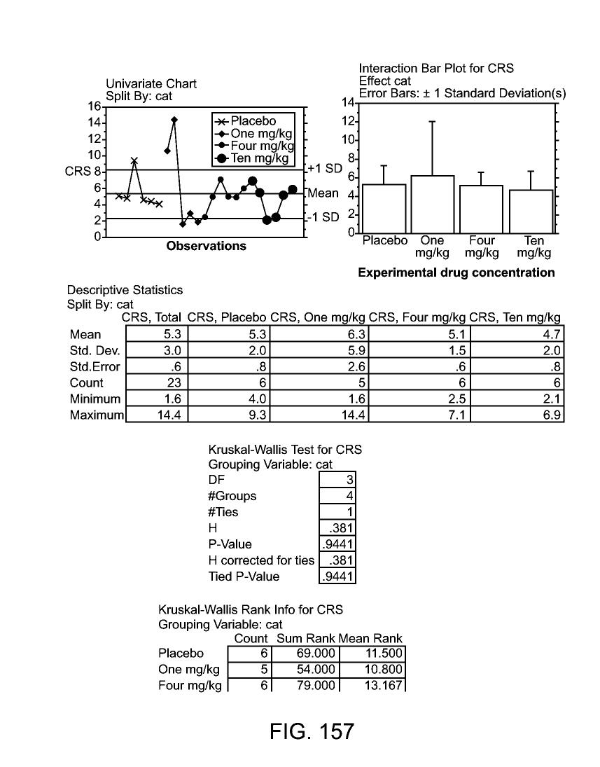

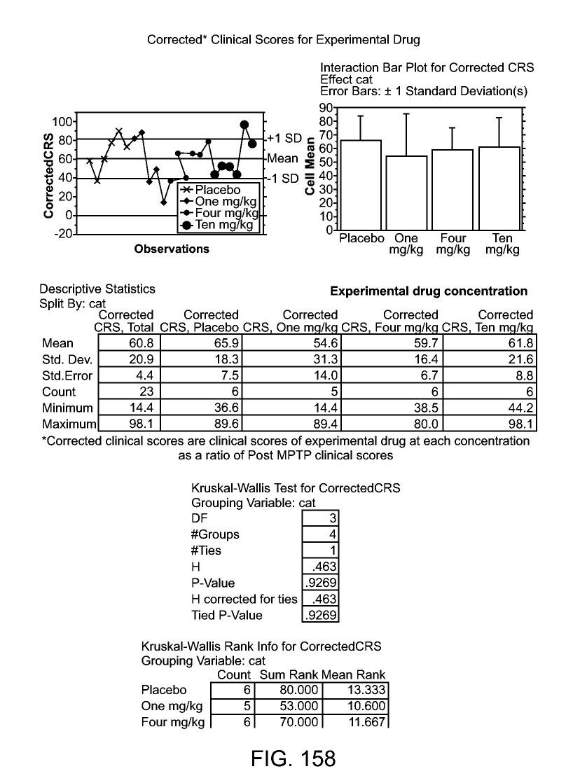

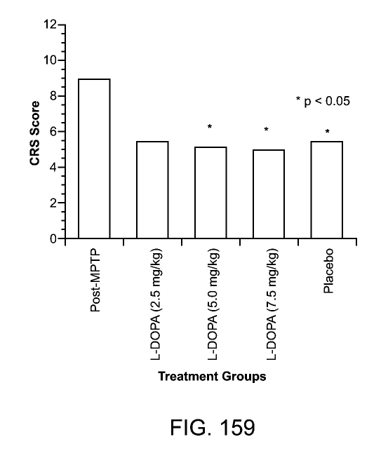

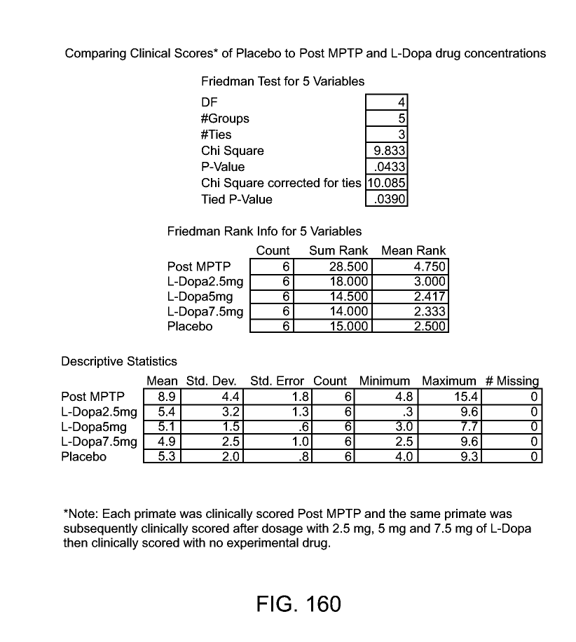

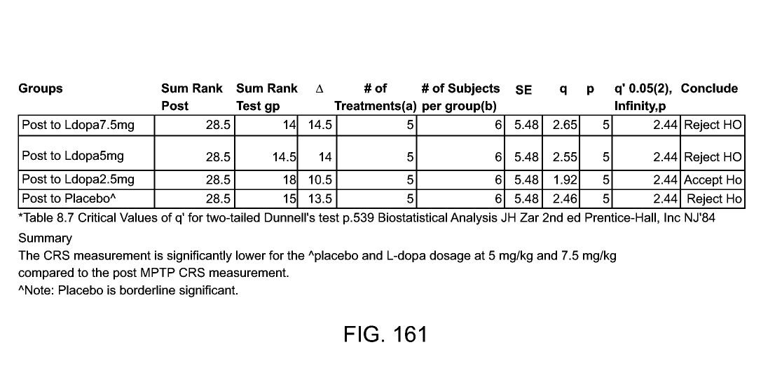

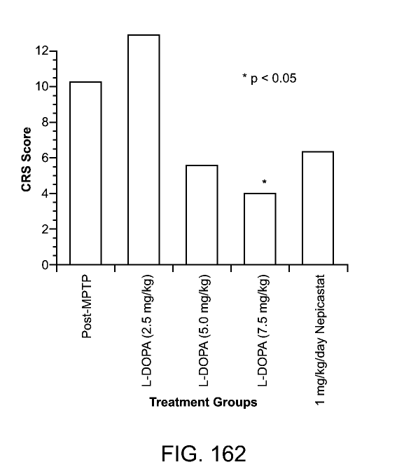

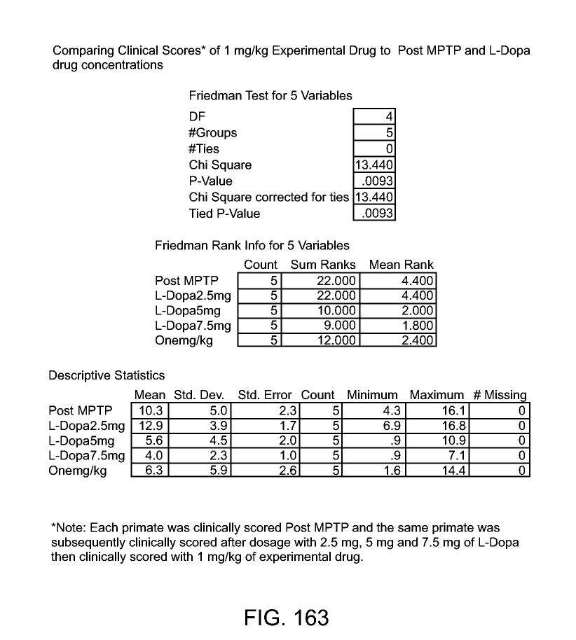

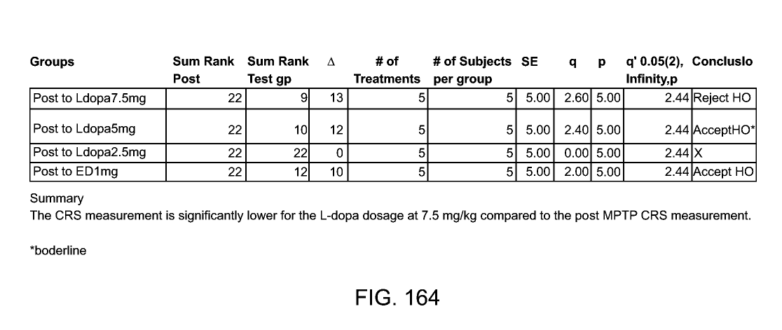

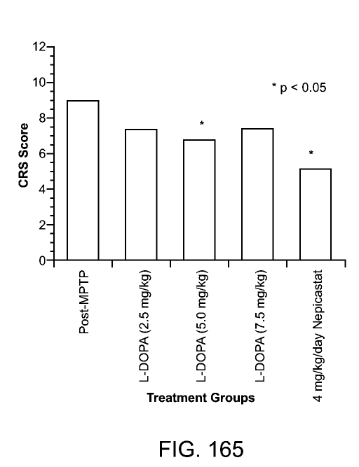

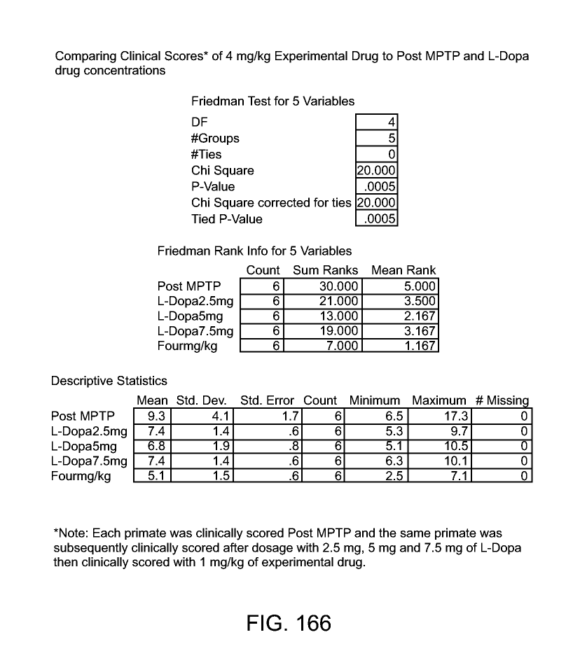

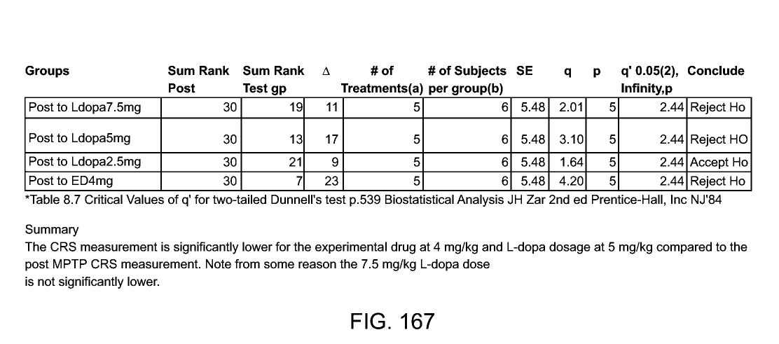

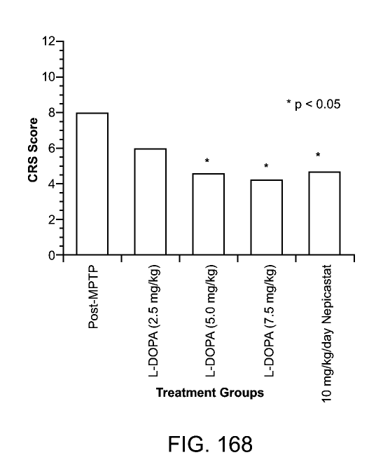

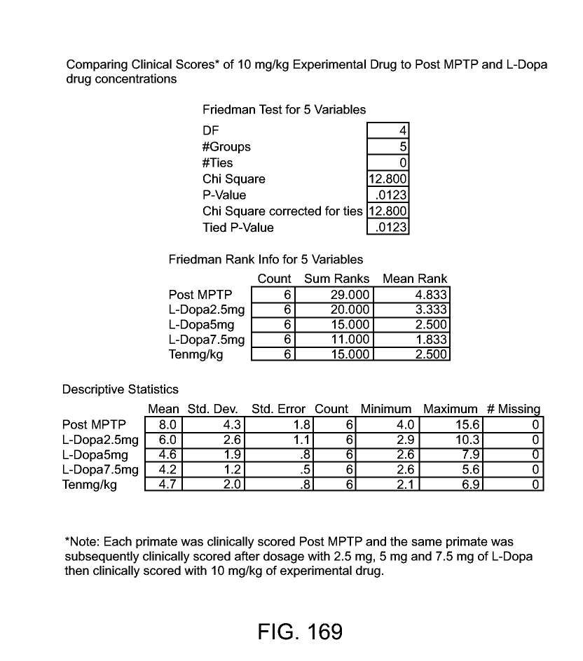

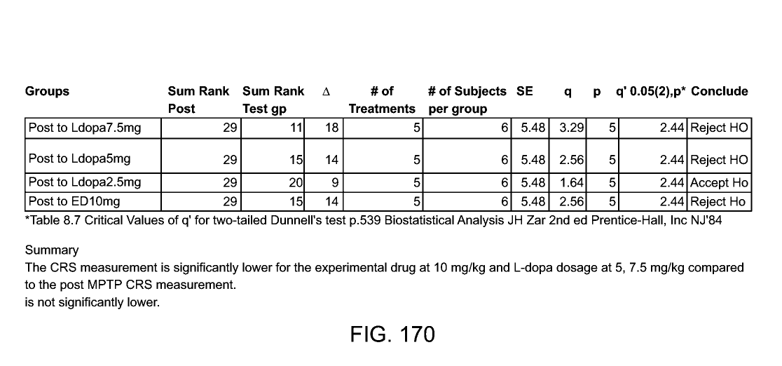

FIGS. 157-170 show comparisons of treatment groups and L-DOPA, Friedman test results, descriptive statistics, and Dunnett's test post hoc analysis.

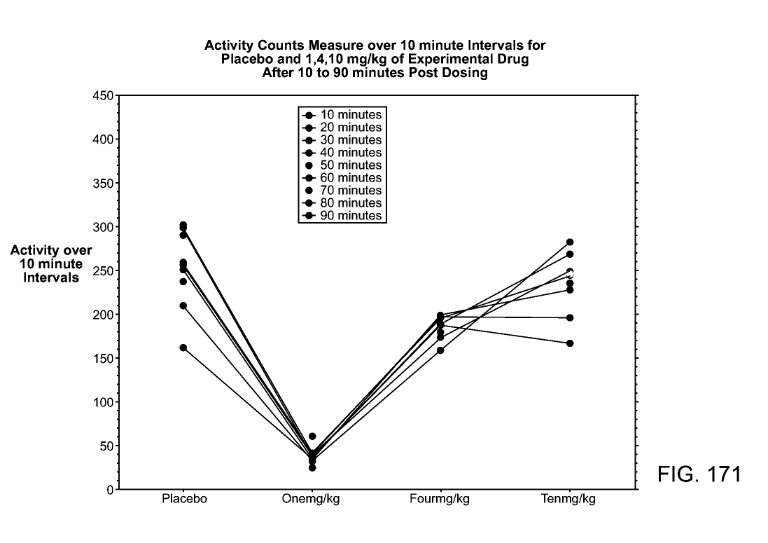

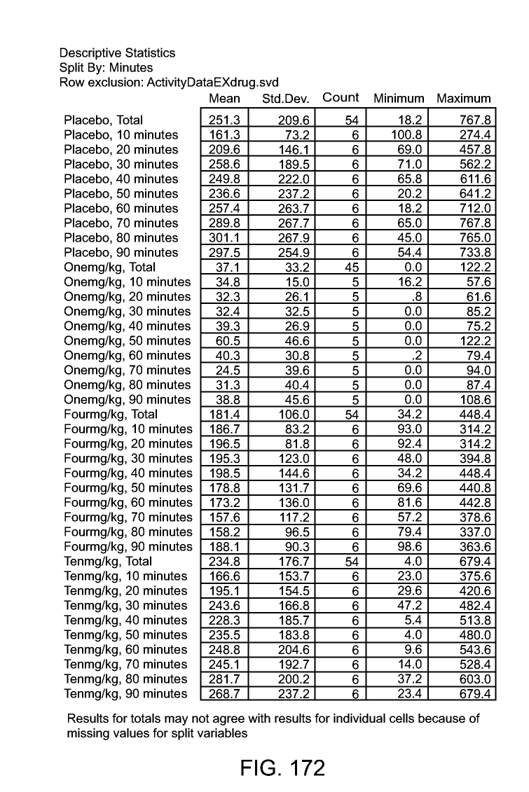

FIGS. 171-172 show the comparison between the activity monitoring of placebo treatment to all other concentrations of nepicastat at time points 10 to 90 minutes post-dosing. Ten-minute intervals were plotted for each drug dose level. There was no difference of drug (nepicastat) treatment at the 4 and 10 mg/kg/day dose level when compared to placebo. At 1 mg/kg/day animals were slower than placebo treatment. Based on a non-pairwise comparative analysis of 4 different treatment groups (1,4, and 10 mg/kg of nepicastat and placebo), nepicastat produced no significant effect in parkinsonian symptoms compared to placebo (water treatment) in the MPTP-lesioned non-human primate model of PD. Based on a pairwise comparative analysis of animals, (animals of the same group examined pre and post treatment), nepicastat at 4 and 10 mg/kg/day concentrations showed a significant effect in parkinsonian symptoms compared to post-MPTP lesioning, (pre-treatment evaluation). Placebo had a borderline significant effect. Using the same pairwise comparison, 5 and 7.5 mg/kg of L-Dopa demonstrated a significant effect when compared to the post-MPTP lesioned state in all groups with the exception of Group B (no effect at 5 mg/kg L-Dopa) and Group C (no effect at 7.5 mg/kg L-Dopa) animals. However, 2.5 mg/kg of L-Dopa demonstrated no significant effect.

This study also demonstrated that, a pairwise analysis, which reduces animal to animal variability by comparing the same animal pre- and post-treatment, is better suited for detecting a significant drug effect than a non-pairwise study design when a small number of animals is used.

### Example 24

Male, spontaneous hypertensive rats (280-345 g; Charles River Labs, Kingston, N.Y.) were fasted overnight then anesthetized with ether. A femoral artery and femoral vein were cannulated with PE50 tubing for recording of blood pressure and administration of compounds, respectively. Animals were then placed in MAYO restrainers and their feet loosely taped to the restrainer. Heparinized saline (50 units sodium heparin/ml) was used to maintain patency of each cannula throughout the experiment. The following parameters were continuously recorded using Modular Instruments MI² BioReport™ software installed on an IBM personal computer: mean arterial pressure (MAP), heart rate (HR), and the change from baseline for each parameter at specified time points in the experiment.

All compounds were dissolved on the day of use. nepicastat was dissolved in deionized water (vehicle) to a free base concentration of 1 mg/ml. Oral dosing volume for nepicastat or vehicle was 10 ml/kg. SCH-23390 was dissolved in saline (vehicle) to a free base concentration of 0.2 mg/ml. Nepicastat or saline were administered intravenously as a bolus in a volume of 1.0 ml/kg followed by 0.2 ml flush of isotonic saline.

Following surgical preparation, each animal was allowed a minimum one hour recovery period. Animals were randomly assigned to four treatment groups: vehicle (iv)/vehicle (po); vehicle (iv)/nepicastat (po); SCH-23390 (iv)/vehicle (po); or SCH-23390 (iv)/nepicastat (po). Once animals were stabilized (minimum one hour), baseline blood pressure and heart rate was determined by taking an average of each parameter over a 15 min period of time. Once baseline blood pressure and heart rate were established, animals were dosed intravenously with either SCH-23390 (200 μg/kg) or vehicle (saline, 1 ml/kg). Fifteen minutes later, animals were orally dosed with either nepicastat (10 mg/kg) or vehicle (deionized water, 10 ml/kg).

Recorded parameters were measured 15 min prior to intravenous dosing to establish baseline blood pressure and heart rate. Recorded parameters were then measured at 5, 10, and 15 min following intravenous administration of SCH-23390 or vehicle. Following oral administration of nepicastat or vehicle, recorded parameters were measured at 15, 30, 60, 90, 120, 150, 180, 210, and 240 min.

At the end of the experiment, each animal was anesthetized with halothane and euthanized via decapitation. The cortex, left ventricle (apex), and mesenteric artery were dissected out, weighed, and fixed in 0.4 M perchloric acid. Tissues were then frozen in liquid nitrogen and stored at −70° C. Biochemical analysis will be performed on these tissues at a later date to determine catecholamine levels (specifically, dopamine and norepinephrine). Assay results will be reported at a later date. Blood pressure and heart rate were analyzed separately. The change from baseline for blood pressure and heart rate were analyzed by an analysis of variance (ANOVA) with effects for treatment, time, and their interaction. This analysis was performed both for the post-iv time period and for the post-oral time period. Further analyses were performed at each time by an ANOVA with a main effect for time. Pairwise comparisons were performed following each ANOVA by Fisher's LSD strategy with a Bonferroni correction when the overall treatment effect was not significant.

An additional analysis was performed to compare the baseline means of each treatment group by an ANOVA with a main effect for treatment and subsequent pairwise comparisons. Comparisons of SCH-23390 (iv)/Vehicle (po) vs. Vehicle (iv)/Vehicle (po), Vehicle (iv)/nepicastat (po) vs. Vehicle (iv)/Vehicle (po), and SCH-23390 (iv)/nepicastat(po) vs. Vehicle (iv)/nepicastat (po) were made.

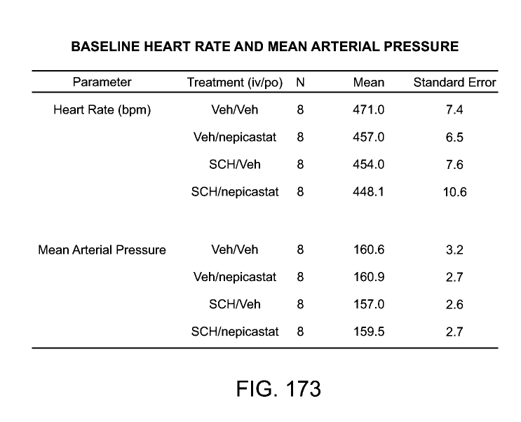

There were no significant differences in baseline heart rate or mean arterial pressure between treatment groups (FIG. 173).

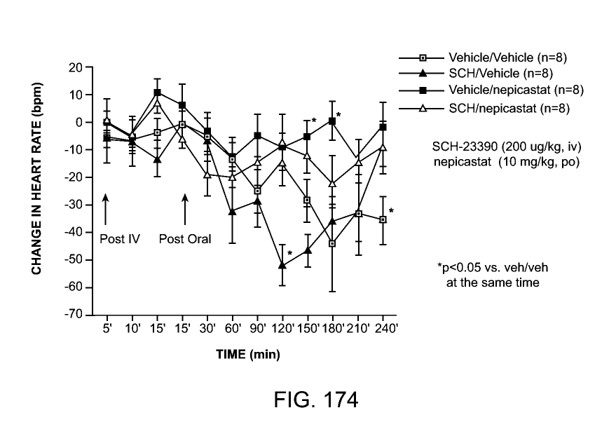

Intravenous treatment with SCH-23390 resulted in a significant decrease (p<0.05) in heart rate during the post-oral period at 120 min and 240 min compared to vehicle control (FIG. 174). Nepicastat did not decrease the heart rate as much as observed in vehicle treated animals. This was statistically significant (p<0.05) at 150 and 180 min post dose (FIG. 174). The large variability in heart rate observed over the course of this experiment should be noted.

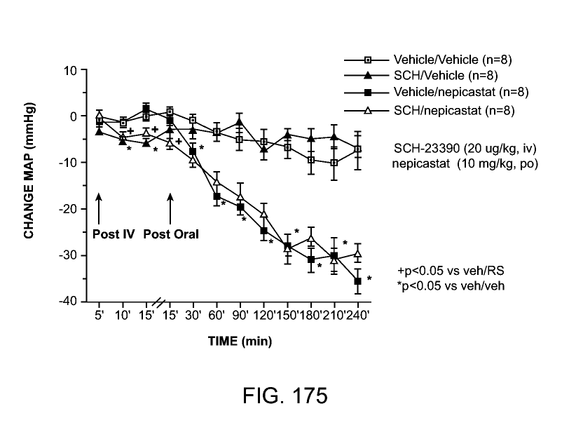

Intravenous administration of SCH-23390 produced a small (5±1 mmHg) yet significant decrease (p<0.05) in mean arterial pressure compared to animals that received vehicle during the 15 min post-iv period (FIG. 175). Oral treatment with nepicastat caused a significant decrease (p<0.05) in mean arterial pressure by 30 min post dose which continued for the duration of the experiment (FIG. 175). Pretreatment with SCH-23390 did not significantly attenuate the antihypertensive effects observed with nepicastat administration alone (FIG. 175).

### Example 25

Male Crl:COBS(WI)BR rats of 15 weeks old were used. Twenty-four rats were chronically implanted with telemetry implants (TA11PA-C40, Data Sciences, Inc., St. Paul, Minn.) for measurement of arterial blood pressure, heart rate and motor activity. The rat was anesthetized with pentobarbital sodium (60 mg/kg, ip) and its abdomen shaved. Under aseptic conditions, an incision was made on midline. The abdominal aorta was exposed, and cannulated with the catheter of a telemetry transmitter unit. After the transmitter was sutured to the abdominal musculature, the skin was closed. Each rat was allowed to recover for at least 2 weeks before being subjected to drug administration. Three days prior to the start of the experiment, the rats were randomly divided into 4 treatment groups: Vehicle (p.o.), Hydralazine (10 mg/kg, p.o.), nepicastat (30 mg/kg, p.o.), nepicastat (100 mg/kg, p.o.).

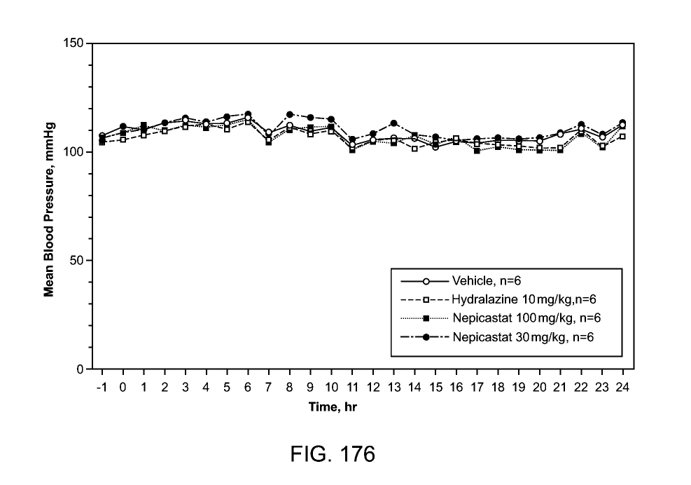

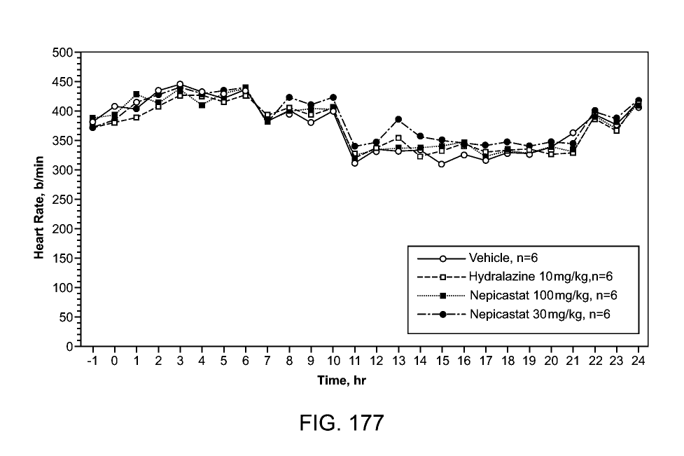

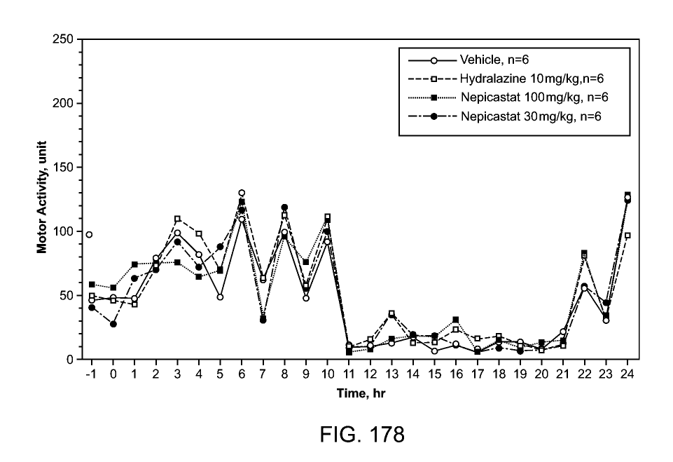

Systolic blood pressure (SBP), diastolic blood pressure (DBP), mean blood pressure (MBP), heart rate (HR), and motor activity (MA) were monitored. After the pre-dose values for these parameters were established, respective groups of rats received a 7 day daily treatment of vehicle, nepicastat or hydralazine. Typical pre-dose data on MBP, HR and MA were presented on FIGS. 176-178.

Both nepicastat and hydralazine were prepared in water with traces of Tween 80. All doses were given orally to the rat in 10 ml/kg and were expressed as free base equivalents.

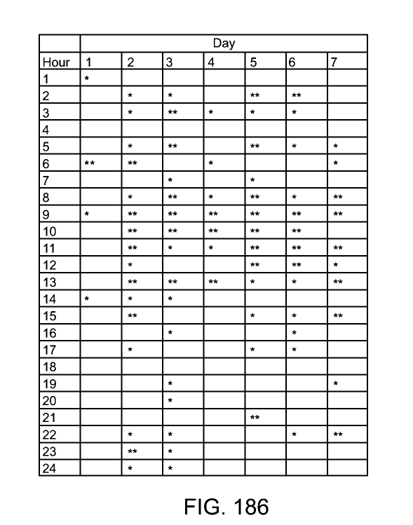

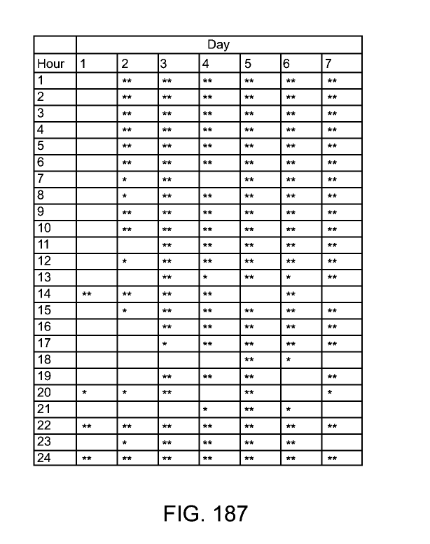

A computerized data collection system was used to continuously collect data on SBP, DBP, MBP, HR, and MA. Data on each rat were collected every 5 min. for 10 sec. These were then averaged hourly and standard errors of the mean (SE) calculated. In this report, only data on MBP, HR and MA were presented. For clarity, the SE bars and indications of significance levels were omitted from the figures (see FIG. 186 for the significance levels for each time point on MBP for rats treated with rufinamide and FIG. 187 for rats treated with hydralazine). Body weights were recorded daily.

All values were expressed as means±SEM. Statistical significance was defined as a p level of less than 0.05.

Data on MBP, HR and MA were analyzed separately. Each analysis was done on 26 time points measured each day. A two-way ANOVA with main effects for treatment and time and their interaction was used. If an overall treatment effect or a significant interaction was detected, a series of one-way ANOVA at each time point would be performed. The pairwise comparisons at each time point were performed using Dunn's procedure. If no overall treatment effect was detected, then the pairwise difference from control would be performed by adjusting the critical value using a Bonferroni adjustment.

For body weight, a two-way ANOVA with respect to the changes from pre-dose was used to analyze overall effects for treatment, day, and treatment by day interaction. Then a one-way ANOVA was performed for each day, and pairwise comparisons for the drug-treated groups to the vehicle controls were made using Dunn's procedure and Fisher's LSD strategy to adjust for multiple comparisons.

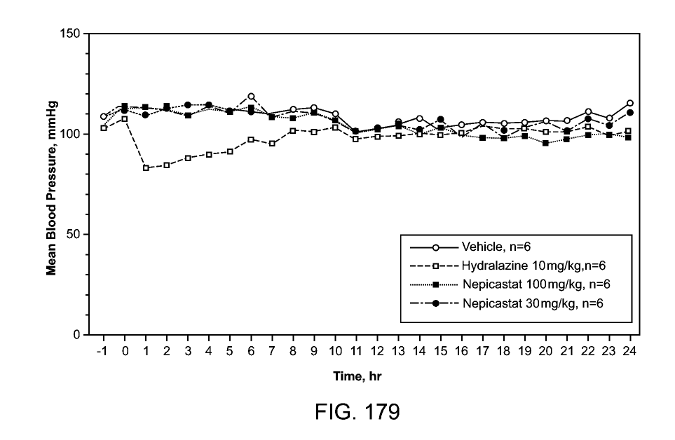

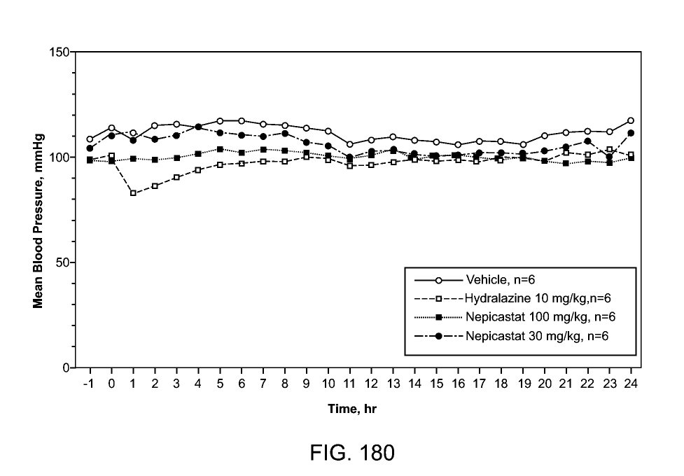

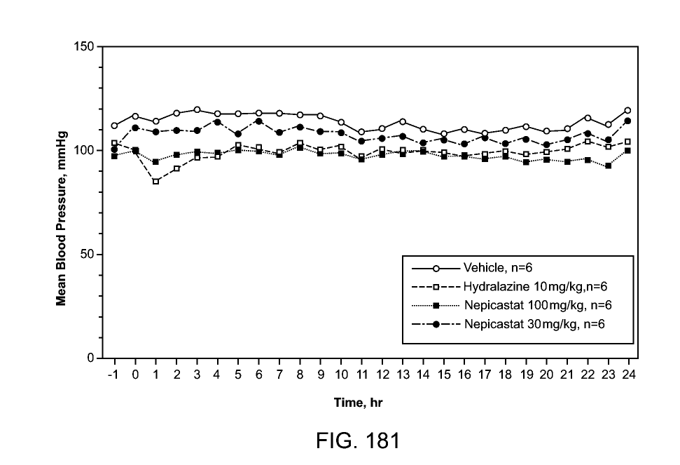

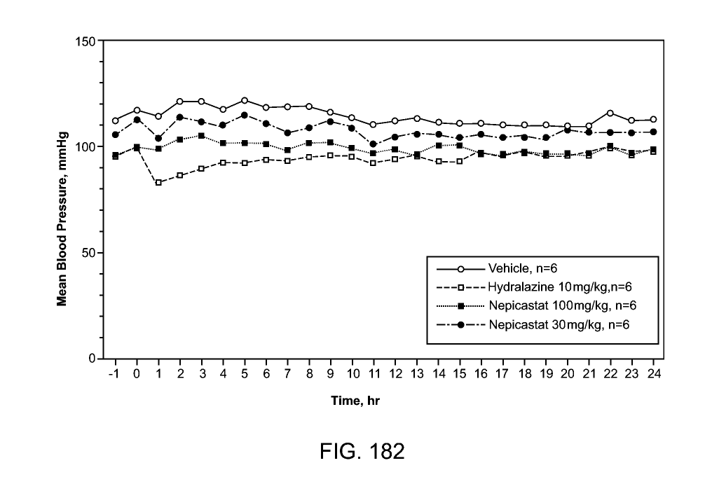

Oral administration of nepicastat at 30 mg/kg (all doses expressed hereafter are po) tended to slowly lower blood pressure but did not induce a consistent hypotensive effect on day 1 (FIG. 179). As the effect progressed, a peak hypotensive effect of −10 mmHg was observed on day 2 at hour 13 (FIG. 180). Similar degrees of antihypertensive effects were induced throughout the study. At 100 mg/kg, the compound induced a peak antihypertensive response of −11 mmHg 22 hr after dosing on day 1 (p<0.01; FIG. 179). MBP continued to decrease and reached its nadir of approximately −17 mmHg on day 3 (p<0.01; FIG. 181). The MBP remained low throughout the study (see FIG. 182, day 7).

Hydralazine at 10 mg/kg caused an immediate hypotensive effect which subsided in 10 hr (FIG. 179). A maximal decrease of −24 mmHg (p<0.01) in MBP was observed within 1 hr after dosing on day 1 (FIG. 179). Similar transient hypotensive effects were observed throughout the study (see FIGS. 179-182).

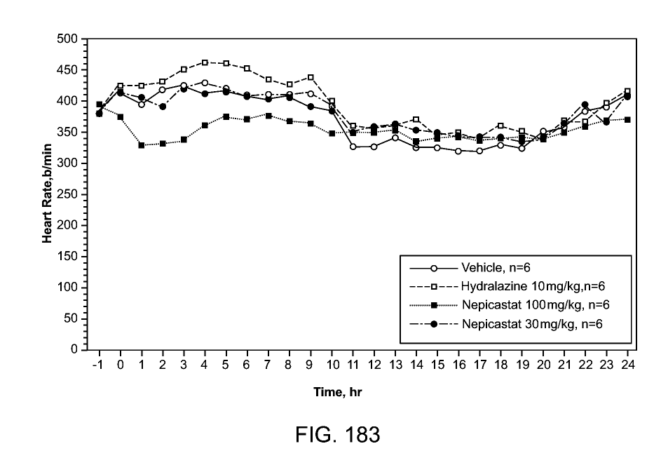

Nepicastat at 30 and 100 mg/kg, did not consistently affect HR on day 1. On day 2, however, Nepicastat at 100 mg/kg caused a bradycardic response of −100 b/mm 3 hours after dosing (FIG. 183). Significant but less pronounced bradycardic responses were observed on days 3-7.

In comparison, hydralazine at 10 mg/kg induced varying degrees of tachycardia throughout the study (see FIG. 183).

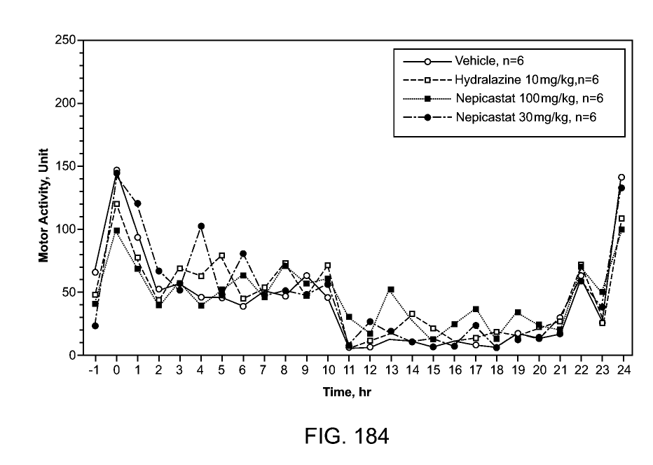

Throughout the study, none of the drug treatments showed a consistent effect on MA (for example, see FIG. 184, day 3).

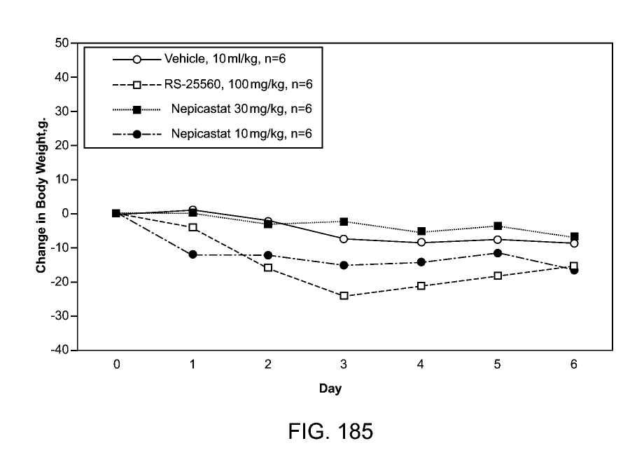

Compared to that treated with vehicle, none of the drug treatments had any effect on body weights (p<0.05; FIG. 185). Although treatment with nepicastat at 100 mg/kg tended to decrease body weight on day 3, it was not statistically significant.

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method of treating a patient diagnosed with post-traumatic stress disorder, comprising administering to the patient a therapeutically effective amount of Compound A.

2. The method of claim 1, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

3. The method of claim 2, wherein the at least one other agent is a SSRI selected from paroxetine, sertraline, citalopram, escitalopram, and fluoxetine.

4. The method of claim 2, wherein the at least one other agent is a SNRI selected from duloxetine, mirtazapine, and venlafaxine.

5. The method of claim 2, wherein the at least one other agent is a NRI selected from bupropion and atomoxetine.

6. The method of claim 2, wherein the at least one other agent is the dopamine β-hydroxylase inhibitor disulfuram.

7. The method of claim 1, wherein the patient has abnormal brain levels of at least one catecholamine.

8. The method of claim 1, wherein the Compound A reduces dopamine β hydroxylase activity in the brain of the patient.

9. The method of claim 1, wherein the Compound A modulates brain levels of at least one catecholamine in the patient.

10. The method of claim 1, wherein the Compound A reduces stress associated with memory recall in the patient.

11. The method of claim 1, wherein the Compound A reduces at least one of the frequency and intensity of at least one sign of the post-traumatic stress disorder in the patient.

12. The method of claim 1, wherein the patient is a child or an adolescent.

13. The method of claim 12, wherein the Compound A reduces at least one of the frequency and intensity of at least one sign or symptom of the post-traumatic stress disorder in the patient, wherein the sign or symptom is selected from disorganized or agitated behavior, repetitive play that expresses aspects of the trauma, frightening dreams which lack recognizable content, and trauma-specific reenactment.

14. The method of claim 1, wherein the Compound A reduces the incidence of at least one disorder comorbid with post-traumatic stress disorder selected from drug abuse, alcohol abuse, and depression in the patient.

15. A method of treating post-traumatic stress disorder in a patient comprising:
diagnosing the patient with post-traumatic stress disorder;
administering to the patient a therapeutically effective amount of Compound A;
assessing at least one of sign, symptom, and symptom cluster of post-traumatic stress disorder; and
determining that the post-traumatic stress syndrome is improved if the Compound A reduces at least one of sign, symptom, and symptom cluster of post-traumatic stress disorder.

16. The method of claim 15, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

17. The method of claim 15, wherein the Compound A reduces at least one of the frequency and intensity of at least one sign of the post-traumatic stress disorder in the patient.

18. The method of claim 15, wherein the Compound A reduces at least one of the frequency and intensity of at least one symptom of the post-traumatic stress disorder in the patient.

19. The method of claim 15, wherein the Compound A reduces at least one of the frequency and intensity of at least one symptom cluster of the post-traumatic stress disorder in the patient, wherein the symptom cluster is selected from re-experiencing/intrusion, avoidance/numbing, and hyperarousal.

20. A method of improving resilience in a patient comprising administering a therapeutically effective amount of Compound A.

21. The method of claim 20, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

22. The method of claim 20, wherein the Compound A reduces at least one of the frequency and intensity of at least one sign of the post-traumatic stress disorder in the patient.

23. A method of diagnosing post-traumatic stress disorder in a patient comprising:
administering to the patient a therapeutically effective amount of Compound A and
assessing at least one of sign, symptom, or symptom cluster of post-traumatic stress disorder; and
diagnosing post-traumatic stress disorder in the patient if the Compound A reduces at least one of sign, symptom, and symptom cluster of post-traumatic stress disorder.

24. The method of claim 23, wherein the patient is a child, adolescent, or adult.
